(19) United States
(12) Patent Application Publication
(10) Pub. No.: US 2010/0173935 A1
(43) Pub. Date: Jul. 8, 2010

(54) Muscarinic Receptor Agonists that are Effective in the Treatment of Pain, Alzheimer's Disease and Schizophrenia

(75) Inventors: Yun-Xing Cheng, Saint Laurent (CA); Shujuan Jin, Saint Laurent (CA); Mehrnaz Pourashraf, Saint Laurent (CA); Miroslaw Tomaszewski, Saint Laurent (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 12/303,641

(22) PCT Filed: Jun. 8, 2007
(86) PCT No.: PCT/SE07/00556

§ 371 Date: Dec. 5, 2008

Related U.S. Application Data
(60) Provisional application No. 60/812,208, filed on Jun. 9, 2006.

(51) Int. Cl. A61K 31/454; C07D 401/14; C07D 405/14; C07D 451/04; A61K 31/46; A61P 25/28; A61P 25/18; A61P 25/22; A61P 25/24; A61P 29/00

(52) U.S. Cl. 514/304; 546/201; 514/323; 546/199; 514/322; 546/126

(57) ABSTRACT

Compounds of Formula IA, or pharmaceutically acceptable salts thereof: IA wherein G¹, G², G³, G⁴, R¹, R², X, Y, Z and n are as defined in the specification as well as salts and pharmaceutical compositions including the compounds are prepared. They are useful in therapy, in particular in the management of pain.

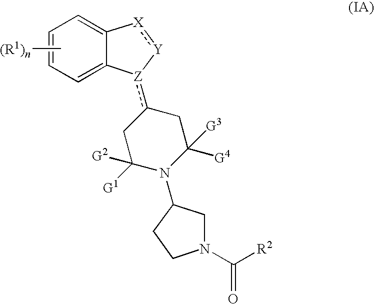

### BACKGROUND OF THE INVENTION

1. Field of the Invention

The present invention relates to agonists of muscarinic receptors. The present invention also provides compositions comprising such agonists, and methods therewith for treating muscarinic receptor mediated diseases. Particularly, the present invention is related to compounds that may be effective in treating pain, Alzheimer's disease, and/or schizophrenia.

2. Discussion of Relevant Technology

The neurotransmitter acetylcholine binds to two types of cholinergic receptors: the ionotropic family of nicotinic receptors and the metabotropic family of muscarinic receptors. Muscarinic receptors belong to the large superfamily of plasma membrane-bound G protein coupled receptors (GPCRs) and show a remarkably high degree of homology across species and receptor subtype. These M1-M5 muscarinic receptors are predominantly expressed within the parasympathetic nervous system which exerts excitatory and inhibitory control over the central and peripheral tissues and participate in a number of physiologic functions, including heart rate, arousal, cognition, sensory processing, and motor control.

Muscarinic agonists such as muscarine and pilocarpine, and antagonists, such as atropine have been known for over a century, but little progress has been made in the discovery of receptor subtype-selective compounds, thereby making it difficult to assign specific functions to the individual receptors. See, e.g., DeLapp, N. et al., “Therapeutic Opportunities for Muscarinic Receptors in the Central Nervous System,” J. Med. Chem., 43(23), pp. 4333-4353 (2000); Hulme, E. C. et al., “Muscarinic Receptor Subtypes,” Ann. Rev. Pharmacol. Toxicol., 30, pp. 633-673 (1990); Caulfield, M. P. et al., “Muscarinic Receptors-Characterization, Coupling, and Function,” Pharmacol. Ther., 58, pp. 319-379 (1993); Caulfield, M. P. et al., International Union of Pharmacology. XVII. Classification of Muscarinic Acetylcholine Receptors,” Pharmacol. Rev., 50, pp. 279-290 (1998).

The Muscarinic family of receptors is the target of a large number of pharmacological agents used for various diseases, including leading drugs for COPD, asthma, urinary incontinence, glaucoma, schizophrenia, Alzheimer's (AchE inhibitors), and Pain.

For example, direct acting muscarinic receptor agonists have been shown to be antinociceptive in a variety of animal models of acute pain (Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992; Capone F., Aloisi A. M., Carli G., Sacerdote P., Pavone F. Oxotremorine-induced modifications of the behavioral and neuroendocrine responses to formalin pain in male rats. Brain Res. 830:292-300, 1999).

A few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999; Lee E. J., Sim J. Y, Park J. Y., Hwang J. H., Park P. H., Han S. M. Intrathecal carbachol and clonidine produce a synergistic antiallodynic effect in rats with a nerve ligation injury. Can J Anaesth 49:178-84, 2002). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side-effects include excessive salivation and sweating, enhanced gastrointestinal motility, and bradycardia among other adverse events. These side-effects are associated with the ubiquitous expression of the muscarinic family of receptors throughout the body.

### DESCRIPTION OF THE EMBODIMENTS

To date, five subtypes of muscarinic receptors (M1-M5) have been cloned and sequenced from a variety of species, with differential distributions in the body.

Therefore, it was desirable to provide molecules would permit selective modulation, for example, of muscarinic receptors controlling central nervous function without also activating muscarinic receptors controlling cardiac, gastrointestinal or glandular functions.

There is also a need for methods for treating muscarinic receptor-mediated diseases.

There is also a need for modulators of muscarinic receptors that are selective as to subtypes M1-M5.

The term “Cₘ₋ₙ” or “Cₘ₋ₙ group” refers to any group having m to n carbon atoms.

The term “alkyl” refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “cycloalkyl” refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “cycloalkenyl” refers to a monovalent ring-containing hydrocarbon radical having at least one carbon-carbon double bond and comprising at least 3 up to about 12 carbon atoms.

The term “aryl” refers to a monovalent hydrocarbon radical having one or more polyunsaturated carbon rings having aromatic character, (e.g., 4n+2 delocalized electrons) and comprising 5 up to about 14 carbon atoms.

The term “heterocycle” refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” refers to a radical derived from a heterocycle by removing one or more hydrogens therefrom.

The term “heterocyclyl” refers a monovalent radical derived from a heterocycle by removing one hydrogen therefrom.

The term “heterocyclylene” refers to a divalent radical derived from a heterocycle by removing two hydrogens therefrom, which serves to links two structures together.

The term “heteroaryl” refers to a heterocyclyl having aromatic character.

The term “heterocylcoalkyl” refers to a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as C₃₋₆heterocycloalkyl.

The term “heteroarylene” refers to a heterocyclylene having aromatic character.

The term “heterocycloalkylene” refers to a heterocyclylene that does not have aromatic character.

The term “six-membered” refers to a group having a ring that contains six ring atoms.

The term “five-membered” refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiacliazolyl, 1,2,4-oxadiazolyl, 1,3,4-triazolyl, 1,3,4-thiadiazolyl, and 1,3,4 oxadiazolyl.

Additionally, heterocycyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” refers to radicals of the general formula O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“RT” or “rt” means room temperature.

In one aspect, an embodiment of the invention provides a compound of Formula I, a pharmaceutically acceptable salt thereof, diastereomers, enantiomers, or mixtures thereof:

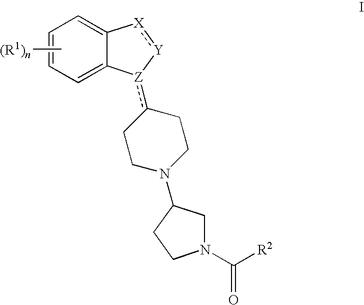

wherein

is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₅heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₅heterocycloalkyl-C₁₋₃alkoxy, C₃₋₅heterocycloalkyl-C₁₋₃alkyl, C₃₋₆cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

n is 1, 2, 3 or 4;

each R is independently hydrogen, C₁₋₆alkyl, C₂₋₆alkenyl or halogenated C₁₋₆alkyl; and

X, Y and Z are independently selected from C(═O), NH, N—CH₃, N, C, CH₂, and CH, wherein at least one of X, Y and Z is selected from NH, N—CH₃ and N; wherein at most one of X, Y and Z is C(═O); and wherein Z is not C(═O).

In a further particular embodiment, R² is selected from hydrogen, C₁₋₆alkyl, C₂₋₆alkenyl, C₁₋₆alkoxy, C₁₋₆alkylamino, di-C₁₋₆alkylamino and benzyloxy, wherein said C₁₋₆alkyl, C₂₋₆alkenyl, C₁₋₆alkoxy, C₁₋₆alkylamino, di-C₁₋₆alkylamino and benzyloxy are optionally substituted by one or more groups selected from amino, halogen, hydroxy, C₁₋₆alkoxy and —CN.

In an even further embodiment, R² is selected from hydrogen, C₁₋₄alkyl, C₁₋₄alkoxy, C₁₋₄alkylamino, di-C₁₋₄alkylamino and benzyloxy.

In another embodiment, R¹ is selected from hydrogen, halogen, methyl, ethyl, —CN, —C(═O)—NH₂, —CO₂CH₃, —CO₂H, hydroxyl, methoxy, trifluoromethyl, FCH₂—, F₂CH—, and CHF₂O—.

In another embodiment, n is 1.

In another embodiment, Z is selected from N, C and CH.

In a further embodiment, Y is selected from N and C(═O).

In an even further embodiment, X is selected from NH and N—CH₃.

In another embodiment, the invention provides a compound of formula II, a pharmaceutically acceptable salt thereof, diastereomer, enantiomer, or mixture thereof:

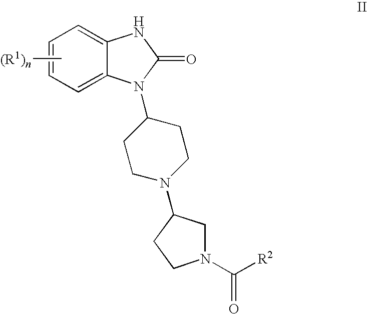

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₅heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₅heterocycloalkyl-C₁₋₃alkoxy, C₃₋₅heterocycloalkyl-C₁₋₃alkyl, C₃₋₆cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

each R is independently hydrogen, C₁₋₆alkyl, C₂₋₆alkenyl or halogenated C₁₋₆alkyl.

In a particular embodiment, R¹ of formula II is independently selected from hydrogen, halogen, C₁₋₃alkyl, —CN, —C(═O)—OH, —C(═O)—NH₂, hydroxy, methoxy, ethoxy, trifluoromethyl, FCH₂—, F₂CH—, and CHF₂O—.

In another particular embodiment, R¹ of formula II is selected from hydrogen halogen, —CN and C₁₋₃alkyl.

In a further particular embodiment, R² of formula II is selected from hydrogen, C₁₋₆alkyl, C₂₋₆alkenyl, C₁₋₆alkoxy, C₁₋₆alkylamino, di-C₁₋₆alkylamino and benzyloxy, wherein said C₁₋₆alkyl, C₂₋₆alkenyl, C₁₋₆alkoxy, C₁₋₆alkylamino, di-C₁₋₆alkylamino and benzyloxy are optionally substituted by one or more groups selected from amino, halogen, hydroxy, C₁₋₆alkoxy and —CN.

In an even further embodiment, R² of formula II is selected from hydrogen, C₁₋₄alkyl, C₁₋₄alkoxy, C₁₋₄alkylamino, di-C₁₋₄alkylamino and benzyloxy.

In another embodiment, the invention provides a compound of formula III, a pharmaceutically acceptable salt thereof, diastereomer, enantiomer, or mixture thereof:

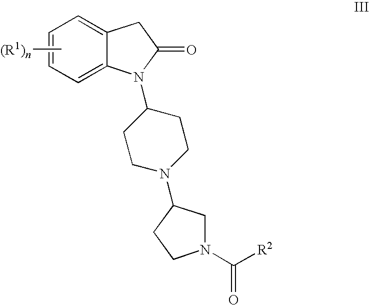

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₅heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₅heterocycloalkyl-C₁₋₃alkoxy, C₃₋₅heterocycloalkyl-C₁₋₃alkyl, C₃₋₆cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆alkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

each R is independently hydrogen, C₁₋₆alkyl, C₂₋₆alkenyl or halogenated C₁₋₆alkyl.

In a particular embodiment, R¹ of formula III is independently selected from hydrogen, halogen, C₁₋₃alkyl, —CN, —C(═O)—OH, —C(═O)—NH₂, hydroxy, methoxy, ethoxy, trifluoromethyl, FCH₂—, F₂CH—, and CHF₂O—.

In another particular embodiment, R¹ of formula III is selected from hydrogen halogen, —CN and C₁₋₃alkyl.

In a further particular embodiment, R² of formula III is selected from hydrogen, C₁₋₆alkyl, C₂₋₆alkenyl, C₁₋₆alkoxy, C₁₋₆alkylamino, di-C₁₋₆alkylamino and benzyloxy, wherein said C₁₋₆alkyl, C₂₋₆alkenyl, C₁₋₆alkoxy, C₁₋₆alkylamino, di-C₁₋₆alkylamino and benzyloxy are optionally substituted by one or more groups selected from amino, halogen, hydroxy, C₁₋₆alkoxy and —CN.

In an even further embodiment, R² of formula III is selected from hydrogen, C₁₋₄alkyl, C₁₋₄alkoxy, C₁₋₄alkylamino, di-C₁₋₄alkylamino and benzyloxy.

In another embodiment, the invention provides a compound of formula IV, a pharmaceutically acceptable salt thereof, diastereomer, enantiomer, or mixture thereof:

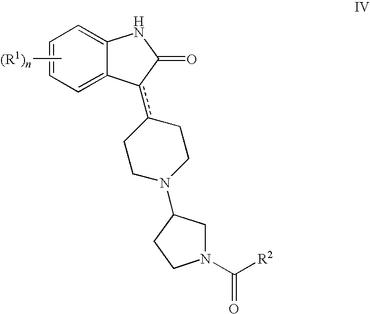

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, C₆₋₁₀aryl, and C₂₋₉heteroaryl;

R² is selected from hydrogen, C₁₋₆aryl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₅heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₅heterocycloalkyl-C₁₋₃alkoxy, C₃₋₅heterocycloalkyl-C₁₋₃alkyl, C₃₋₆cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkyl-carbonyl, C₁₋₆alkylaminocarbonyl, C₆₋₁₀aryl, C₂₋₉heteroaryl, C₃₋₅heterocycloalkyl, C₆₋₁₀aryl-C₁₋₃alkyl, C₂₋₉heteroaryl-C₁₋₃alkyl, C₃₋₅heterocycloalkyl-C₁₋₃alkyl, C₃₋₆cycloalkyl, and C₃₋₆cycloalkyl-C₁₋₃alkyl are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

each R is independently hydrogen, C₁₋₆alkyl, C₂₋₆alkenyl or halogenated C₁₋₆alkyl.

In a particular embodiment, R¹ of formula IV is independently selected from hydrogen, halogen, C₁₋₃alkyl, —CN, —C(═O)—OH, —C(═O)—NH₂, hydroxy, methoxy, ethoxy, trifluoromethyl, FCH₂—, F₂CH—, and CHF₂O—.

In another particular embodiment, R¹ of formula IV is selected from hydrogen halogen, —CN and C₁₋₃alkyl.

In a further particular embodiment, R² of formula IV is selected from hydrogen, C₁₋₆alkyl, C₂₋₆alkenyl, C₁₋₆alkoxy, C₁₋₆alkylamino, di-C₁₋₆alkylamino and benzyloxy, wherein said C₁₋₆alkyl, C₂₋₆alkenyl, C₁₋₆alkoxy, C₁₋₆alkylamino, di-C₁₋₆alkylamino and benzyloxy are optionally substituted by one or more groups selected from amino, halogen, hydroxy, C₁₋₆alkoxy and —CN.

In an even further embodiment, R² of formula IV is selected from hydrogen, C₁₋₄alkyl, C₁₋₄alkoxy, C₁₋₄alkylamino, di-C₁₋₄alkylamino and benzyloxy.

In another embodiment, the invention provides a compound of formula IA, a pharmaceutically acceptable salt thereof, diastereomer, enantiomer, or mixture thereof:

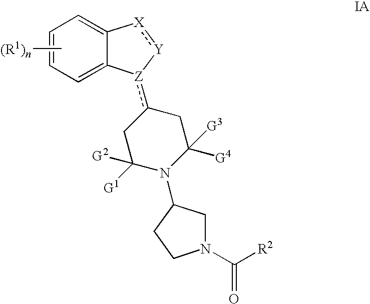

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl. C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

G¹, G², G³ and G⁴ are independently selected from H and methyl; or two of G¹, G², G³ and G⁴ are linked together to form a C₁₋₄alkylene, and the other two are independently selected from H and methyl;

n is 1, 2, 3 or 4;

each R is independently hydrogen, C₁₋₆alkyl, C₂₋₆alkenyl or halogenated C₁₋₆alkyl; and

X, Y and Z are independently selected from C(═O), NH, N—CH₃, N, C, CH₂, and CH, wherein at least one of X, Y and Z is selected from NH, N—CH₃ and N; wherein at most one of X, Y and Z is C(═O); and wherein Z is not C(═O).

In a particular embodiment, R² of formula IA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

In a particular embodiment, R² of formula IA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

In a particular embodiment, R¹ of formula IA is selected from hydrogen, halogen, methyl, ethyl, —CN, —C(═O)—NH₂, —CO₂CH₃, —CO₂H, hydroxyl, methoxy, ethoxy, isopropoxy, trifluoromethyl, FCH₂—, F₂CH—, CHF₂O—, and CF₃O—.

In a particular embodiment, Z of formula IA is selected from N, C and CH.

In a particular embodiment, Y of formula IA is selected from N and C(═O).

In a particular embodiment, X of formula IA is selected from CH₂, NH and N—CH₃.

In a particular embodiment, G¹, G², G³ and G⁴ of formula IA are independently selected from —H and methyl.

In a particular embodiment, G¹, G², G³ and G⁴ of formula IA are —H.

In a particular embodiment, G² and G³ of formula IA are linked together to form an ethylene, and G¹ and G⁴ of formula IA are independently selected from —H and methyl.

In a particular embodiment, G² and G³ of formula IA are linked together to form a bond, and G¹ and G⁴ of formula IA are independently selected from —H and methyl.

In a further embodiment, the invention provides a compound of formula IIA, a pharmaceutically acceptable salt thereof, diastereomer, enantiomer, or mixture thereof:

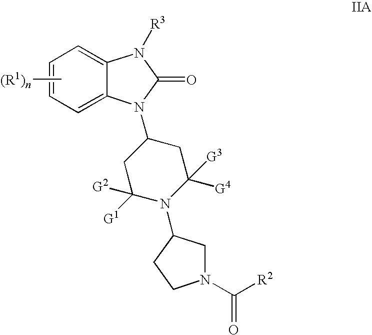

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

R³ is H or C₁₋₄ alkyl;

G¹, G², G³ and G⁴ are independently selected from H and methyl; or two of G¹, G², G³ and G⁴ are linked together to form a C₁₋₄alkylene, and the other two are independently selected from H and methyl; and

each R is independently hydrogen, C₁₋₆alkyl, C₂₋₆alkenyl or halogenated

In another embodiment, R¹ of formula IIA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, F₂CH—, CF₃O—, and CHF₂O—.

In a particular embodiment, R¹ of formula IIA is selected from hydrogen halogen, —CN, methoxy and C₁₋₃alkyl.

In a particular embodiment, R² of formula IIA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

In a particular embodiment, R² of formula IIA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

In another particular embodiment, R³ is H or methyl.

In an even more particular embodiment, R³ is H.

In an even further embodiment, the invention provides a compound of formula IIIA, a pharmaceutically acceptable salt thereof, diastereomer, enantiomer, or mixture thereof:

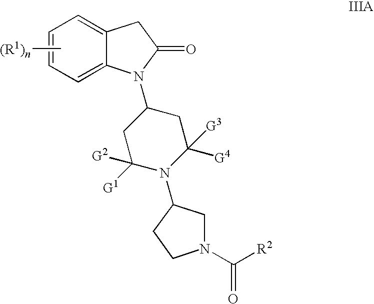

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

G¹, G², G³ and G⁴ are independently selected from H and methyl; or two of G¹, G², G³ and G⁴ are linked together to form a C₁₋₄alkylene, and the other two are independently selected from H and methyl; and

each R is Independently hydrogen, C₁₋₆alkyl, C₂₋₆alkenyl or halogenated C₁₋₆alkyl.

In a particular embodiment, R¹ of formula IIIA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, CF₃O—, and CHF₂O—.

In a particular embodiment, R¹ of formula IIIA is selected from hydrogen, halogen, —CN, methoxy and C₁₋₃alkyl.

In a particular embodiment, R² of formula IIIA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

In a particular embodiment, R² of formula IIIA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

In another embodiment, the invention provides a compound of formula IVA, a pharmaceutically acceptable salt thereof, diastereomer, enantiomer, or mixture thereof:

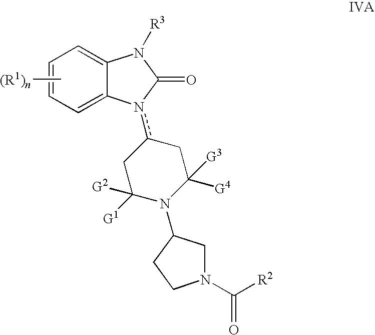

wherein

R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;

R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkoxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;

R³ is H or C₁₋₄ alkyl;

G¹, G², G³ and G⁴ are independently selected from H and methyl; or two of G¹, G², G³ and G⁴ are linked together to form a C₁₋₄alkylene, and the other two are independently selected from H and methyl; and

each R is independently hydrogen, C₁₋₆alkyl, C₂₋₆alkenyl or halogenated C₁₋₆alkyl.

In a particular embodiment, R¹ of formula IVA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, F₂CH—, CF₃O—, and CHF₂O—.

In a particular embodiment, R¹ of formula IVA is selected from hydrogen halogen, —CN, methoxy and C₁₋₃alkyl.

In a particular embodiment, R² of formula IVA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

In a particular embodiment, R² of formula IVA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

In another particular embodiment, R³ of formula IVA is H or methyl.

In an even more particular embodiment, R³ of formula IVA is H.

In a further embodiment, the invention provides a compound selected from
Ethyl 3-[4-(2-oxo-2,3-dihydro-1H-indol-1-yl)piperidin-1-yl]pyrrolidine-1-carboxylate;
Ethyl 3-[4-(2-oxo-2,3-dihydro-1H-benzimidazol-1-yl)piperidin-1-yl]pyrrolidine-1-carboxylate;
Ethyl 3-[4-(5-chloro-2-oxo-2,3-dihydro-1H-benzimidazol-1-yl)piperidin-1-yl]pyrrolidine-1-carboxylate;
Benzyl 3-[4-(2-oxo-2,3-dihydro-1H-benzimidazol-1-yl)piperidin-1-yl]pyrrolidine-1-carboxylate;
t-Butyl 3-[4-(2-oxo-2,3-dihydro-1H-benzimidazol-1-yl)piperidin-1-yl]pyrrolidine-1-carboxylate;
Isopropyl 3-[4-(2-oxo-2,3-dihydro-1H-benzimidazol-1-yl)piperidin-1-yl]pyrrolidine-1-carboxylate;
1-[1-(1-butyrylpyrrolidin-3-yl)piperidin-4-yl]-1,3-dihydro-2H-benzimidazol-2-one;
N,N-dimethyl-3-[4-(2-oxo-2,3-dihydro-1H-benzimidazol-1-yl)piperidin-1-yl]pyrrolidine-1-carboxamide;
1-{1-[1-(3-methylbutanoyl)pyrrolidin-3-yl]piperidin-4-yl}-1,3-dihydro-2H-benzimidazol-2-one;
Ethyl 3-[4-(3-methyl-2-oxo-2,3-dihydro-1H-benzimidazol-1-yl)piperidin-1-yl]pyrrolidine-1-carboxylate;
Ethyl 3-[4-(1H-1,2,3-benzotriazol-1-yl)piperidin-1-yl]pyrrolidine-1-carboxylate;
Ethyl 3-[4-(2-oxo-1,2-dihydro-3H-indol-3-ylidene)piperidin-1-yl]pyrrolidine-1-carboxylate;
Ethyl 3-[4-(2-oxo-2,3-dihydro-1H-indol-3-yl)piperidin-1-yl]pyrrolidine-1-carboxylate;
tert-Butyl (3S)-3-[4-(2-oxo-2,3-dihydro-1H-benzimidazol-1-yl)piperidin-1-yl]pyrrolidine-1-carboxylate;
Ethyl (3S)-3-[4-(2-oxo-2,3-dihydro-1H-benzimidazol-1-yl)piperidin-1-yl]pyrrolidine-1-carboxylate;
Ethyl (3R)-3-[4-(2-oxo-2,3-dihydro-1H-benzimidazol-1-yl)piperidin-1-yl]pyrrolidine-1-carboxylate;
Methyl (3S)-3-[4-(2-oxo-2,3-dihydro-1H-benzimidazol-1-yl)piperidin-1-yl]pyrrolidine-1-carboxylate;
iso-Propyl (3S)-3-[4-(2-oxo-2,3-dihydro-1H-benzimidazol-1-yl)piperidin-1-yl]pyrrolidine-1-carboxylate;
1-{1-[(3S)-1-(cyclopentylcarbonyl)pyrrolidin-3-yl]piperidin-4-yl}-1,3-dihydro-2H-benzimidazol-2-one;
1-(1-{(3S)-1-[(2S)-tetrahydrofuran-2-ylcarbonyl]pyrrolidin-3-yl}piperidin-4-yl)-1,3-dihydro-2H-benzimidazol-2-one;
1-(1-{(3S)-1-[(1-methyl-1H-pyrrol-2-yl)carbonyl]pyrrolidin-3-yl}piperidin-4-yl)-1,3-dihydro-2H-benzimidazol-2-one;
1-(1-{(3S)-1-[4-(2-oxopyrrolidin-1-yl)butanoyl]pyrrolidin-3-yl}piperidin-4-yl)-1,3-dihydro-2H-benzimidazol-2-one;
1-(1-{(3S)-1-[3-(2-oxopyrrolidin-1-yl)propanoyl]pyrrolidin-3-yl}piperidin-4-yl)-1,3-dihydro-2H-benzimidazol-2-one;
1-methyl-3-(1-{(3S)-1-[3-(2-oxopyrrolidin-1-yl)propanoyl]pyrrolidin-3-yl}piperidin-4-yl)-1,3-dihydro-2H-benzimidazol-2-one;
(3S)—N-ethyl-3-[4-(2-oxo-2,3-dihydro-1H-benzimidazol-1-yl)piperidin-1-yl]pyrrolidine-1-carboxamide;
Ethyl (3S)-3-[4-(2-oxo-2,3-dihydro-1H-indol-1-yl)piperidin-1-yl]pyrrolidine-1-carboxylate;
Ethyl (3R)-3-[4-(2-oxo-2,3-dihydro-1H-indol-1-yl)piperidin-1-yl]pyrrolidine-1-carboxylate;
Methyl (3S)-3-[4-(2-oxo-2,3-dihydro-1H-indol-1-yl)piperidin-1-yl]pyrrolidine-1-carboxylate;
1-(1-{(3S)-1-[3-(2-oxopyrrolidin-1-yl)propanoyl]pyrrolidin-3-yl}piperidin-4-yl)-1,3-dihydro-2H-indol-2-one;
Ethyl 3-[3-(2-oxo-2,3-dihydro-1H-benzimidazol-1-yl)-8-azabicyclo[3.2.1]oct-8-yl]pyrrolidine-1-carboxylate;
Ethyl 3-[4-(7-fluoro-2-oxo-2,3-dihydro-1H-benzimidazol-1-yl)piperidin-1-yl]pyrrolidine-1-carboxylate;
Ethyl 3-[4-(5-fluoro-2-oxo-2,3-dihydro-1H-benzimidazol-1-yl)piperidin-1-yl]pyrrolidine-1-carboxylate;
Ethyl 3-[4-(4-fluoro-2-oxo-2,3-dihydro-1H-benzimidazol-1-yl)piperidin-1-yl]pyrrolidine-1-carboxylate;
Ethyl 3-[4-(6-fluoro-2-oxo-2,3-dihydro-1H-benzimidazol-1-yl)piperidin-1-yl]pyrrolidine-1-carboxylate;
(3S) Ethyl 3-[4-(6-fluoro-2-oxo-2,3-dihydro-1H-benzimidazol-1-yl)piperidin-1-yl]pyrrolidine-1-carboxylate;
(3R) Ethyl 3-[4-(6-fluoro-2-oxo-2,3-dihydro-1H-benzimidazol-1-yl)piperidin-1-yl]pyrrolidine-1-carboxylate;
(3S) Ethyl 3-[4-(6-methyl-2-oxo-2,3-dihydro-1H-benzimidazol-1-yl)piperidin-1-yl]pyrrolidine-1-carboxylate;
(3R) ethyl 3-[4-(6-methyl-2-oxo-2,3-dihydro-1H-benzimidazol-1-yl)piperidin-1-yl]pyrrolidine-1-carboxylate;
(3S) Ethyl 3-[4-(6-methoxy-2-oxo-2,3-dihydro-1H-benzimidazol-1-yl)piperidin-1-yl]pyrrolidine-1-carboxylate;
(3R) ethyl 3-[4-(6-methoxy-2-oxo-2,3-dihydro-1H-benzimidazol-1-yl)piperidin-1-yl]pyrrolidine-1-carboxylate;
Ethyl (3S)-3-[4-(6-cyano-2-oxo-2,3-dihydro-1H-benzimidazol-1-yl)piperidin-1-yl]pyrrolidine-1-carboxylate;
Ethyl (3S)-3-[4-(6-chloro-2-oxo-2,3-dihydro-1H-benzimidazol-1-yl)piperidin-1-yl]pyrrolidine-1-carboxylate;
Ethyl (3S)-3-[4-(6-trifluoromethyl-2-oxo-2,3-dihydro-1H-benzimidazol-1-yl)piperidin-1-yl]pyrrolidine-1-carboxylate;
Ethyl (3S)-3-[4-(5-trifluoromethyl-2-oxo-2,3-dihydro-1H-benzimidazol-1-yl)piperidin-1-yl]pyrrolidine-1-carboxylate;
Ethyl (3S)-3-[4-(6-tert-butyl-2-oxo-2,3-dihydro-1H-benzimidazol-1-yl)piperidin-1-yl]pyrrolidine-1-carboxylate;
Ethyl (3S)-3-[4-(5-tert-butyl-2-oxo-2,3-dihydro-1H-benzimidazol-1-yl)piperidin-1-yl]pyrrolidine-1-carboxylate;
Ethyl (3S)-3-[4-(6-trifluoromethoxy-2-oxo-2,3-dihydro-1H-benzimidazol-1-yl)piperidin-1-yl]pyrrolidine-1-carboxylate;
Ethyl (3S)-3-[4-(5-trifluoromethoxy-2-oxo-2,3-dihydro-1H-benzimidazol-1-yl)piperidin-1-yl]pyrrolidine-1-carboxylate;
Ethyl (3S)-3-[4-(5-fluoro-2-oxo-2,3-dihydro-1H-benzimidazol-1-yl)piperidin-1-yl]pyrrolidine-1-carboxylate;
   and pharmaceutically acceptable salts thereof.

It will be understood that when compounds of the present invention contain one or more chiral centers, the compounds of the Invention may exist in, and be isolated as, enantiomeric or diastereomeric forms, or as a racemic mixture. The present invention includes any possible enantiomers, diastereomers, racemates or mixtures thereof, of a compound of Formula I, IA, II, IIA, III, IIIA, IV or IVA. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the present invention may exist as geometrical isomers, for example E and Z isomers of alkenes. The present invention includes any geometrical isomer of a compound of Formula I, IA, II, IIA, III, IIIA, IV or IVA. It will further be understood that the present invention encompasses tautomers of the compounds of the formula I, IA, II, IIA, III, IIIA, IV or IVA.

It will also be understood that certain compounds of the present invention may exist in solvated, for example hydrated, as well as unsolvated forms. It will further be understood that the present invention encompasses all such solvated forms of the compounds of the Formula I, IA, II, IIA, III, IIIA, IV or IVA.

Within the scope of the invention are also salts of the compounds of the Formula I, IA, II, IIA, III, IIIA, IV or IVA. Generally, pharmaceutically acceptable salts of compounds of the present invention may be obtained using standard procedures well known in the art, for example by reacting a sufficiently basic compound, for example an alkyl amine with a suitable acid, for example, HCl or acetic acid, to afford a physiologically acceptable anion. It may also be possible to make a corresponding alkali metal (such as sodium, potassium, or lithium) or an alkaline earth metal (such as a calcium) salt by treating a compound of the present invention having a suitably acidic proton, such as a carboxylic acid or a phenol with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques.

In one embodiment, the compound of Formula I, IA, II, IIA, III, IIIA, IV or IVA above may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

We have now found that the compounds of the invention have activity as pharmaceuticals, in particular as agonists of M1 receptors. More particularly, the compounds of the invention exhibit selective activity as agonist of the M1 receptors and are useful in therapy, especially for relief of various pain conditions such as chronic pain, neuropathic pain, acute pain, cancer pain, pain caused by rheumatoid arthritis, migraine, visceral pain etc. This list should however not be Interpreted as exhaustive. Additionally, compounds of the present invention are useful in other disease states in which dysfunction of M1 receptors is present or implicated. Furthermore, the compounds of the invention may be used to treat cancer, multiple sclerosis, Parkinson's disease, Huntington's chorea, schizophrenia, Alzheimer's disease, anxiety disorders, depression, obesity, gastrointestinal disorders and cardiovascular disorders.

In a particular embodiment, the compounds may be used to treat schizophrenia or Alzheimer's disease.

In another embodiment, the compounds may be used to treat pain.

In another particular embodiment, the compounds may be used to treat neuropathic pain.

Compounds of the invention are useful as immunomodulators, especially for autoimmune diseases, such as arthritis, for skin grafts, organ transplants and similar surgical needs, for collagen diseases, various allergies, for use as anti-tumour agents and anti viral agents.

Compounds of the invention are useful in disease states where degeneration or dysfunction of M1 receptors is present or implicated in that paradigm. This may involve the use of isotopically labeled versions of the compounds of the invention in diagnostic techniques and imaging applications such as positron emission tomography (PET).

Compounds of the invention are useful for the treatment of diarrhea, depression, anxiety and stress-related disorders such as post-traumatic stress disorders, panic disorder, generalized anxiety disorder, social phobia, and obsessive compulsive disorder, urinary incontinence, premature ejaculation, various mental illnesses, cough, lung oedema, various gastro-intestinal disorders, e.g. constipation, functional gastrointestinal disorders such as Irritable Bowel Syndrome and Functional Dyspepsia, Parkinson's disease and other motor disorders, traumatic brain injury, stroke, cardioprotection following miocardial infarction, obesity, spinal injury and drug addiction, including the treatment of alcohol, nicotine, opioid and other drug abuse and for disorders of the sympathetic nervous system for example hypertension.

Compounds of the invention are useful as an analgesic agent for use during general anaesthesia and monitored anaesthesia care. Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

Also within the scope of the invention is the use of any of the compounds according to the Formula I, IA, II, IIA, III, IIIA, IV or IVA above, for the manufacture of a medicament for the treatment of any of the conditions discussed above.

A further aspect of the invention is a method for the treatment of a subject suffering from any of the conditions discussed above, whereby an effective amount of a compound according to the Formula I, IA, II, IIA, III, IIIA, IV or IVA above; is administered to a patient in need of such treatment.

Thus, the invention provides a compound of Formula I, IA, II, IIA, III, IIIA, IV or IVA or pharmaceutically acceptable salt or solvate thereof, as hereinbefore defined for use in therapy.

In a further aspect, the present invention provides the use of a compound of Formula I, IA, II, IIA, III, IIIA, IV or IVA or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The term “therapeutic” and “therapeutically” should be contrued accordingly. The term “therapy” within the context of the present invention further encompasses to administer an effective amount of a compound of the present invention, to mitigate either a pre-existing disease state, acute or chronic, or a recurring condition. This definition also encompasses prophylactic therapies for prevention of recurring conditions and continued therapy for chronic disorders.

The compounds of the present invention are useful in therapy, especially for the therapy of various pain conditions including, but not limited to: acute pain, chronic pain, neuropathic pain, back pain, cancer pain, and visceral pain. In a particular embodiment, the compounds are useful in therapy for neuropathic pain. In an even more particular embodiment, the compounds are useful in therapy for chronic neuropathic pain.

In use for therapy in a warm-blooded animal such as a human, the compound of the invention may be administered in the form of a conventional pharmaceutical composition by any route including orally, intramuscularly, subcutaneously, topically, intranasally, intraperitoneally, intrathoracially, intravenously, epidurally, intrathecally, transdermally, intracerebroventricularly and by injection into the joints.

In one embodiment of the invention, the route of administration may be oral, intravenous or intramuscular.

The dosage will depend on the route of administration, the severity of the disease, age and weight of the patient and other factors normally considered by the attending physician, when determining the individual regimen and dosage level at the most appropriate for a particular patient.

For preparing pharmaceutical compositions from the compounds of this invention, inert, pharmaceutically acceptable carriers can be either solid or liquid. Solid form preparations include powders, tablets, dispersible granules, capsules, cachets, and suppositories.

A solid carrier can be one or more substances, which may also act as diluents, flavoring agents, solubilizers, lubricants, suspending agents, binders, or table disintegrating agents; it can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture in then poured into convenient sized moulds and allowed to cool and solidify.

Suitable carriers are magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art.

Depending on the mode of administration, the pharmaceutical composition will preferably include from 0.05% to 99% w (percent by weight), more preferably from 0.10 to 50% w, of the compound of the invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

Within the scope of the invention is the use of any compound of Formula I, IA, II, IIA, III, IIIA, IV or IVA as defined above for the manufacture of a medicament.

Also within the scope of the inventions the use of any compound of Formula I, IA, II, IIA, III, IIIA, IV or IVA for the manufacture of a medicament for the therapy of pain.

Additionally provided is the use of any compound according to Formula I, IA, II, IIA, III, IIIA, IV or IVA for the manufacture of a medicament for the therapy of various pain conditions including, but not limited to: acute pain, chronic pain, neuropathic pain, back pain, cancer pain, and visceral pain.

A further aspect of the invention is a method for therapy of a subject suffering from any of the conditions discussed above, whereby an effective amount of a compound according to the Formula I, IA, II, IIA, III, IIIA, IV or IVA above, is administered to a patient in need of such therapy.

Additionally, there is provided a pharmaceutical composition comprising a compound of Formula I, IA, II, IIA, III, IIIA, IV or IVA or a pharmaceutically acceptable salt thereof, in association with a pharmaceutically acceptable carrier.

Particularly, there is provided a pharmaceutical composition comprising a compound of Formula I, IA, II, IIA, III, IIIA, IV or IVA or a pharmaceutically acceptable salt thereof, in association with a pharmaceutically acceptable carrier for therapy, more particularly for therapy of pain.

Further, there is provided a pharmaceutical composition comprising a compound of Formula I, IA, II, IIA, III, IIIA, IV or IVA or a pharmaceutically acceptable salt thereof, in association with a pharmaceutically acceptable carrier use in any of the conditions discussed above.

In a further embodiment, a compound of the present invention, or a pharmaceutical composition or formulation comprising a compound of the present invention may be administered concurrently, simultaneously, sequentially or separately with one or more pharmaceutically active compound(s) selected from the following:

(i) antidepressants such as amitriptyline, amoxapine, bupropion, citalopram, clomipramine, desipramine, doxepin duloxetine, elzasonan, escitalopram, fluvoxamine, fluoxetine, gepirone, imipramine, Ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine and equivalents and pharmaceutically active isomer(s) and Metabolite(s) thereof;

(ii) atypical antipsychotics including for example quetiapine and pharmaceutically active isomer(s) and metabolite(s) thereof, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, lithium, loxapine, mesoridazine, olanzapine, paliperidone, perlapine, perphenazine, phenothiazine, phenylbutlypiperidine, pimozide, prochlorperazine, risperidone, quetiapine, sertindole, sulpiride, suproclone, suriclone, thioridazine, trifluoperazine, trimetozine, valproate, valproic acid, zopiclone, zotepine, ziprasidone and equivalents thereof;

(iii) antipsychotics including for example amisuipride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutlypiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, NXY-059, citicoline, crobenetine, desmoteplase, repinotan, traxoprodil and equivalents and pharmaceutically active isomer(s) and metabolite(s) thereof;

(x) over active bladder urinary incontinence therapies including, for example, darafenacin, falvoxate, oxybutynin, propiverine, robaizotan, solifenacin, tolterodine and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combinations employ the compounds of this invention within the dosage range described herein and the other pharmaceutically active compound or compounds within approved dosage ranges and/or the dosage described in the publication reference.

In an even further embodiment, a compound of the present invention, or a pharmaceutical composition or formulation comprising a compound of the present Invention may be administered concurrently, simultaneously, sequentially or separately with one or more pharmaceutically active compound(s) selected from buprenorphine; dezocine; diacetylmorphine; fentanyl; levomethadyl acetate; meptazinol; morphine; oxycodone; oxymorphone; remifentanil; sufentanil; and tramadol.

In a particular embodiment, it may be particularly effective to administrate a combination containing a compound of the invention and a second active compound selected from buprenorphine; dezocine; diacetylmorphine; fentanyl; levomethadyl acetate; meptazinol; morphine; oxycodone; oxymorphone; remifentanil; sufentanil; and tramadol to treat chronic nociceptive pain. The efficacy of this therapy may be demonstrated using a rat FCA-induced heat hyperalgesia model described below.

In a further aspect, the present invention provides a method of preparing the compounds of the present invention.

In one embodiment, the invention provides a process for preparing a compound of Formula I, comprising:

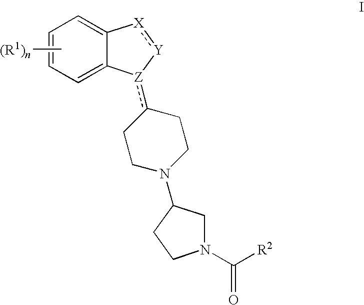

reacting a compound of formula V with a compound of formula VI,

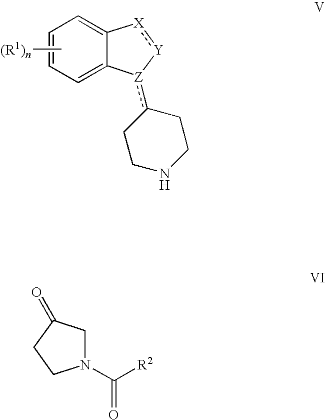

wherein R¹, R², X, Y and Z are defined as above.

Optionally, the step of reacting a compound of formula V with a compound of formula VI is carried out in the presence of a reducing agent, such as NaBH(OAc)₃, NaBH₄ or equivalents thereof.

In another embodiment, the invention provides an intermediate of formula VII,

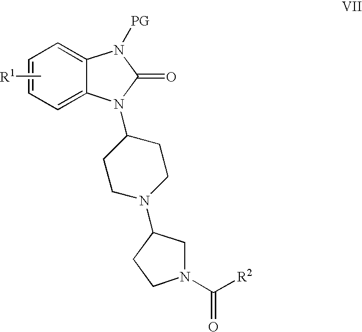

wherein R¹ and R² are defined as above, PG is a protecting group such as —C(═O)═O-t-Bu or C(═O)—OBn. “Bn” stands for benzyl.

In a further embodiment, the invention provides an intermediate of formula VIIA,

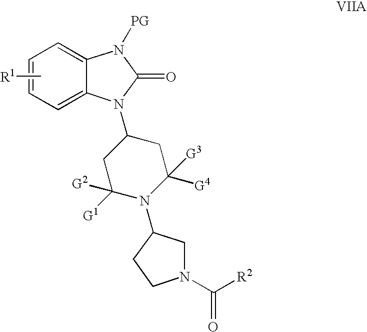

wherein R¹, R², PG, Gⁱ, G², G³ and G⁴ are as defined above.

In an even further embodiment, the invention provides an intermediate of formula VIII,

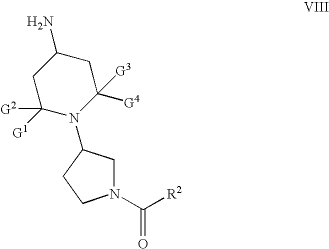

wherein R², G¹, G², G³ and G⁴ are as defined above.

In another embodiment, the invention provides an intermediate of formula IX

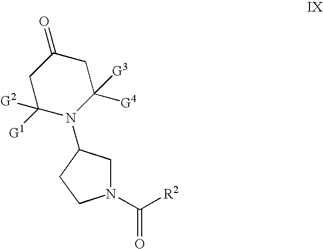

wherein R², G³ and G⁴ are as defined above.

In an even further embodiment, the invention provides a process for preparing a compound of Formula VIII, comprising:

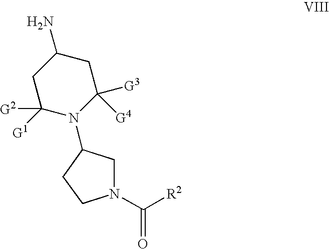

reductive amination of a compound of Formula IX

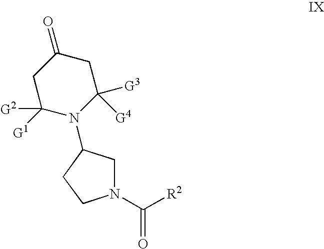

wherein R¹, R², G¹, G², G³ and G⁴ are as defined above. The reduction amination step may be carried out with an amination agent and a reducing agent. The amination agent may be an amine, amine salt such as amino acetate, or other amine containing compounds. The reducing agent may be, for example, NaBH₄, AlH₃, sodium triacetoxyborohydride, or other similar hydride type compounds.

In a further embodiment, the invention provides a method of preparing a compound of formula IIA comprising

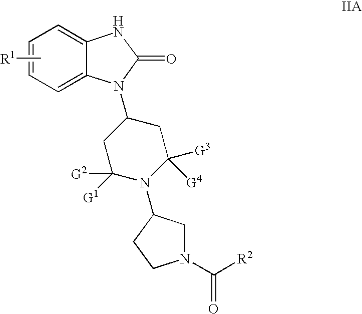

a first of step of reacting a compound of formula IX

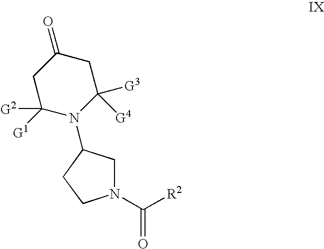

with a compound of formula X in the presence of a reducing agent to form a first product; and

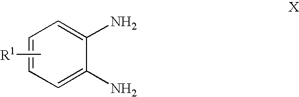

reacting said first product with a phosgene type reagent to form the compound of formula IIA

wherein the reducing agent, R¹, R², G¹, G³ and G⁴ are as defined above. The phosgene type reagent may be, for example, triphosgene, phosgene, or N,N′-carbonyldiimidazole (CDI).

In another embodiment, the invention provides a method of preparing a compound of formula IIA comprising

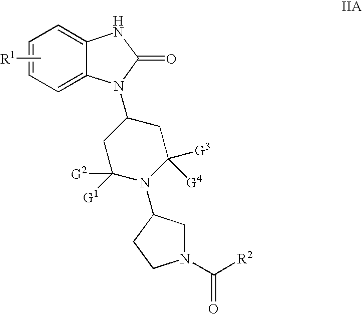

a first step of reacting a compound of formula VIII

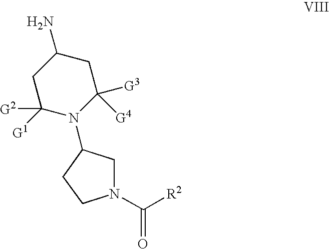

with a compound of formula XI in the presence of a reducing agent to form a first product containing a nitro group; and

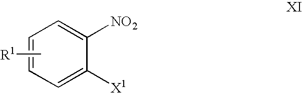

reducing the nitro group of said first product into an amino group to form a second product;

reacting said second product with a phosgene type reagent to form the compound of formula IIA

wherein X¹ is a halogen; the reducing agent, R¹, R², G¹, G², G³ and G⁴ are as defined above. The reduction of the nitro group may be carried out using standard reduction procedures such as hydrogenation with hydrogen in the presence of a transition metal catalyst such as Pd.

Compounds of the present invention may also be prepared according to the synthetic routes as depicted in Schemes 1-8.

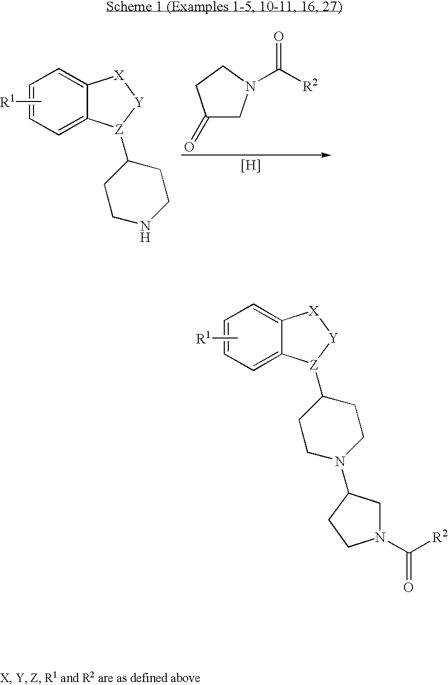

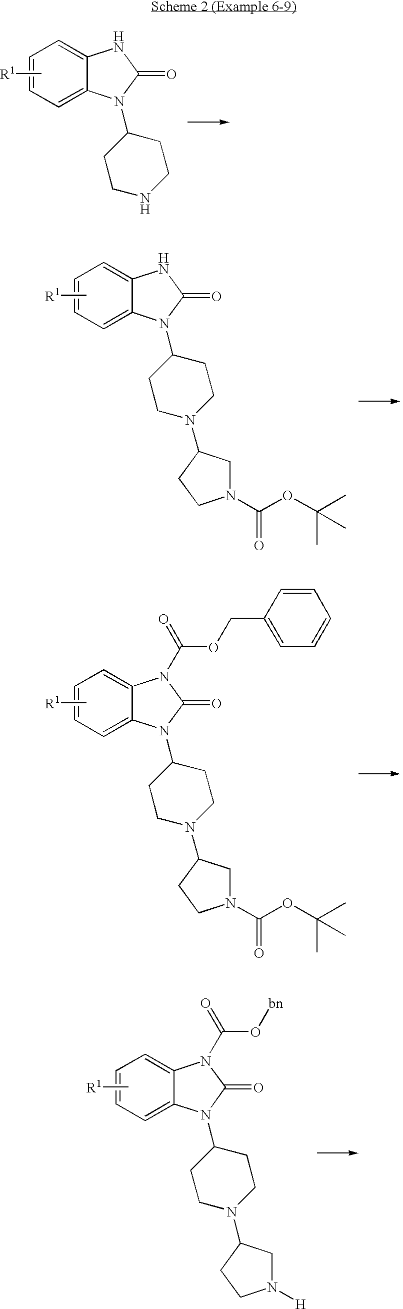

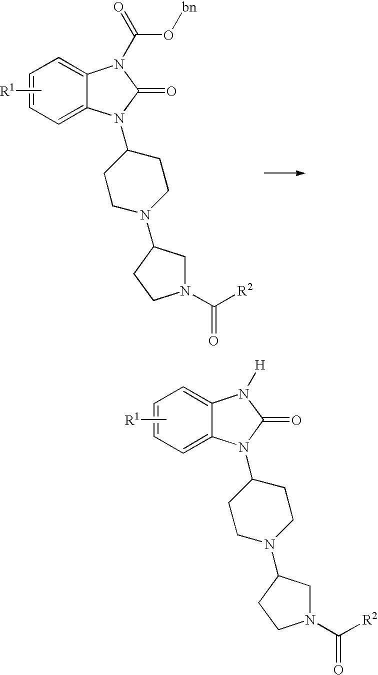

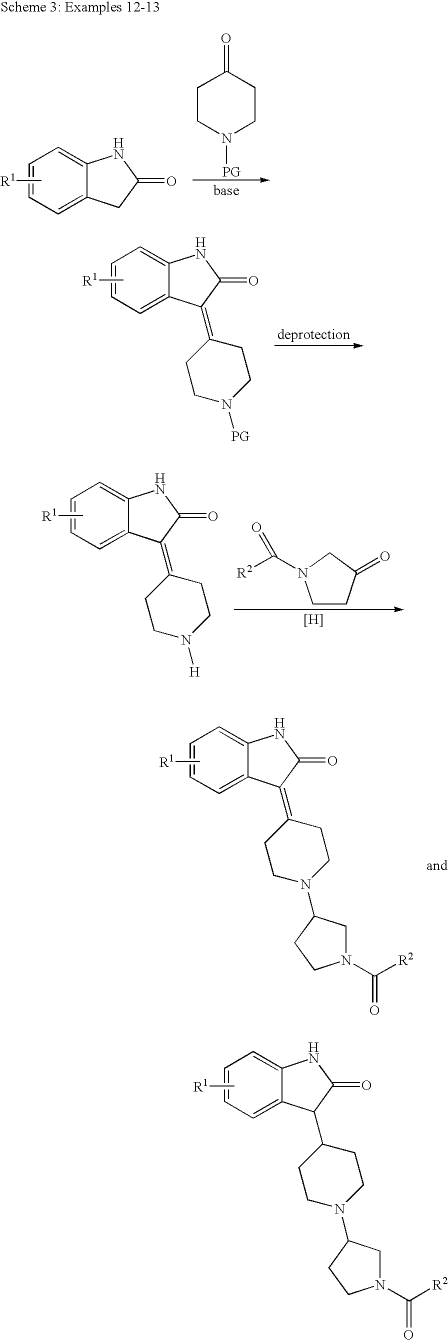

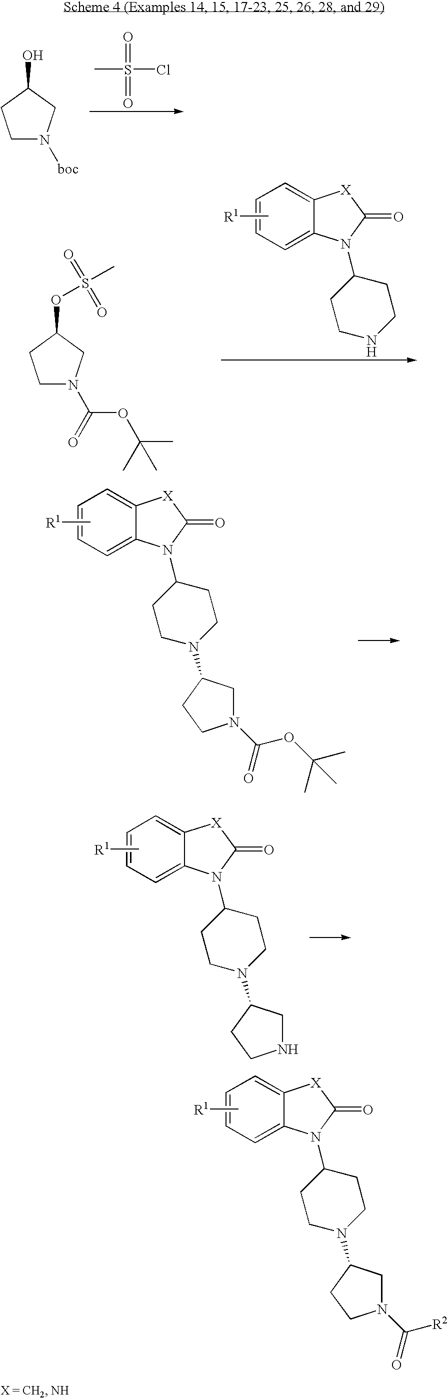

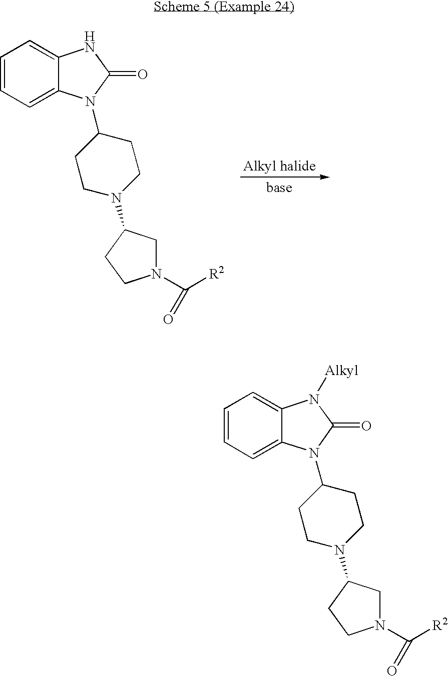

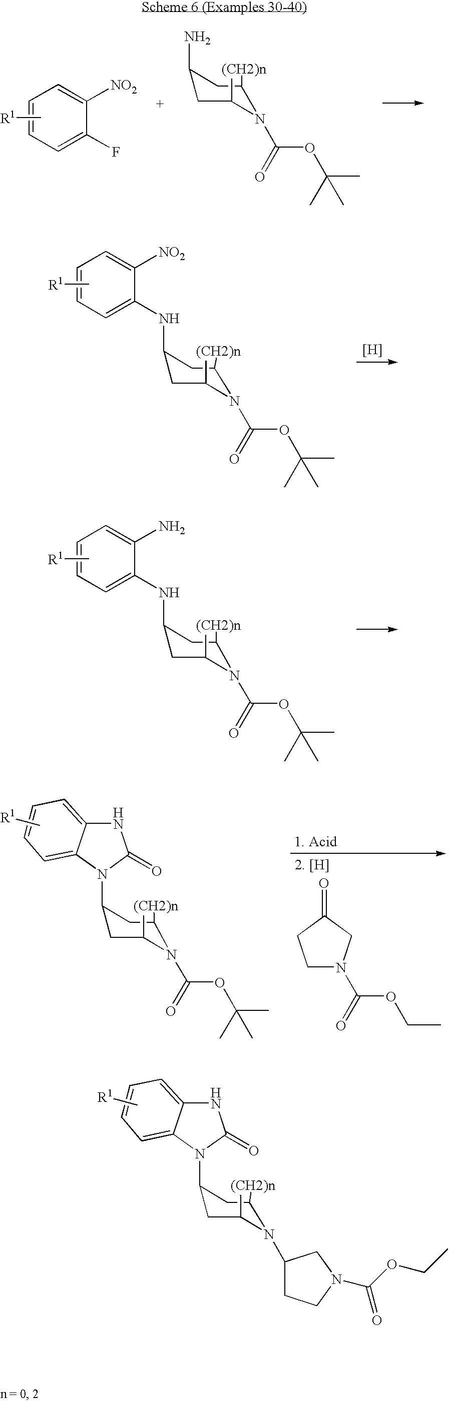

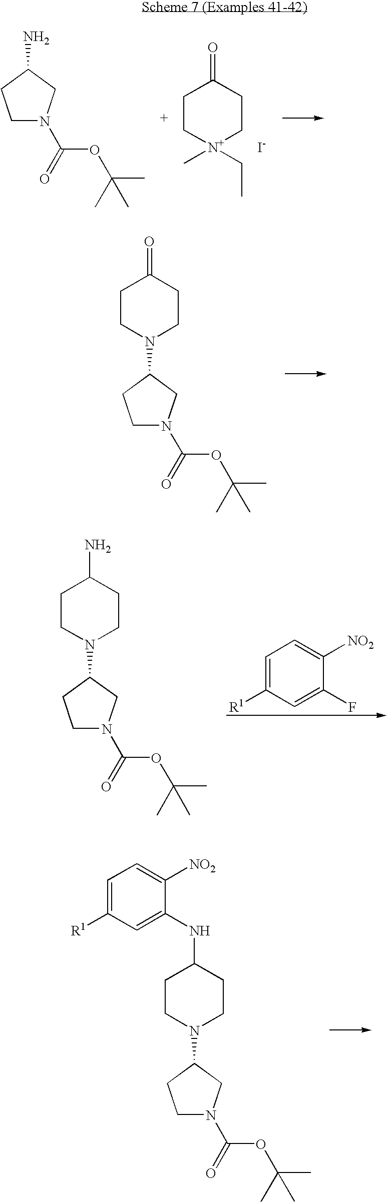

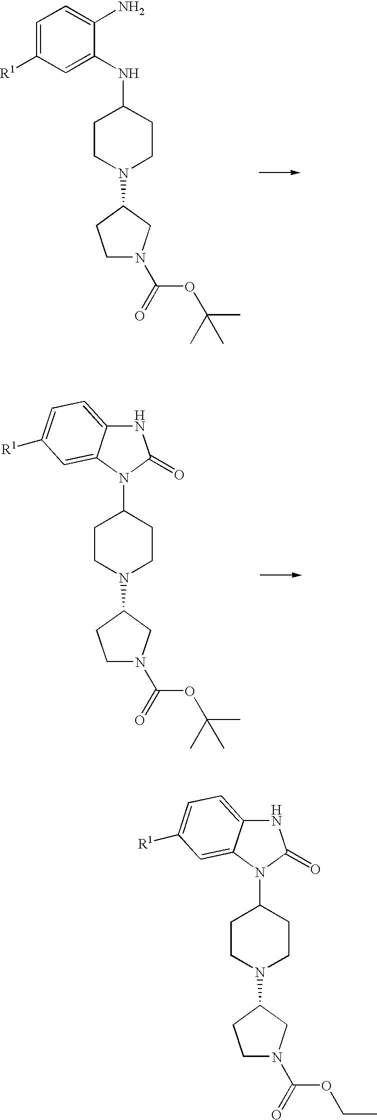

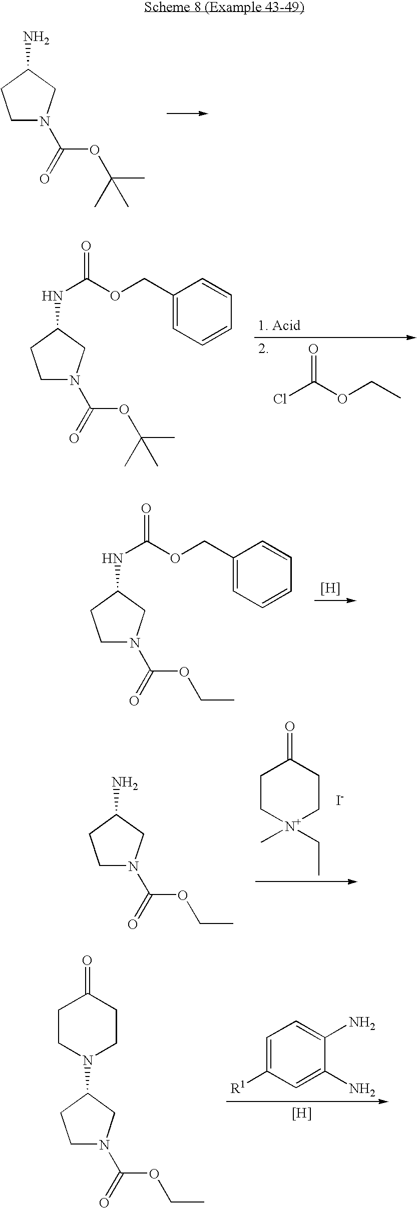

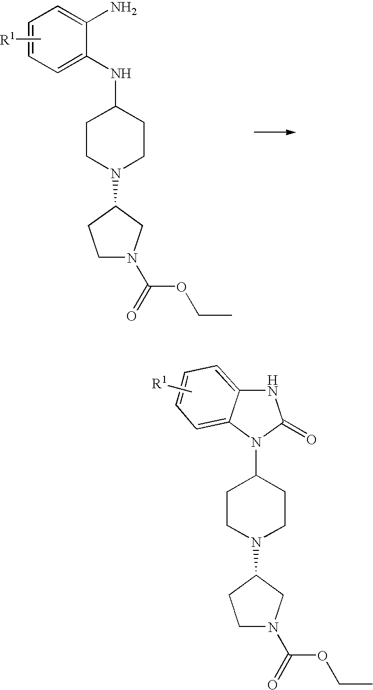

### Biological Evaluation

### Human M1, Rat M1, Human M3 and Human M5 Calcium Mobilization FLIPR™ Assay

The compound activity in the present invention (EC50 or IC₅₀) was measured using a 384 plate-based imaging assay that monitors drug induced intracellular Ca² release in whole cells. Activation of hM1 (human Muscarinic receptor subtype 1, gene bank access NM_{—}000738), rM1 (rat Muscarinic receptor subtype 1, gene bank access NM_{—}080773), hM3 (human Muscarinic receptor subtype 3, gene bank access NM_{—}000740NM_{—}000740) and hM5 (human Muscarinic receptor subtype 5, gene bank access NM_{—}0121258), receptors expressed in CHO cells (chinese hamster ovary cells, ATCC) was quantified in a Molecular Devices FLIPR II™ instrument as an increase in fluorescent signal. Inhibition of hM3 and hM5 by compounds was determined by the decrease in fluorescent signal in response to 20 nM carbachol activation.

CHO cells were plated in 384-black polylysine coated plate (Costar) at 8000 cells/well/50 μl for 24 hours or 4000 cells/well for 48 hours in a humidified incubator (5% CO₂ and 37° C.) in DMEM/F12 medium without selection agent. Prior to the experiment the cell culture medium was removed from the plates by inversion. A loading solution of 30 μl of Hank's balanced salt solution, 10 mM Hepes and 2.5 mM Probenicid at Ph 7.4 (Cat no. 311-520-VL, Wisent) with 2 μM calcium indicator dye (FLUO-3AM, Molecular Probes F14202) was added to each well. Plates were incubated at 37° C. for 60 minutes prior to start the experiment. The incubation was terminated by washing the cells four times in assay buffer, leaving a residual 25 μl buffer per well. Cell plates were then transferred to the FLIPR, ready for compound additions.

The day of experiment, carbachol and compounds were diluted in three-fold concentration range (10 points serial dilution) for addition by FLIPR instrument. For all calcium assays, a baseline reading was taken for 30 seconds followed by the addition of 12.5 μl (25 μl for hM1 and rM1) of compounds, resulting in a total well volume of 37.5 μl (50 μl for hM1 and rM1). Data were collected every 1.6 seconds for 300 seconds. For hM3 and hM5 an additional 12.5 μl of carbachol (20 nM final) was added at 300 seconds. After this addition of carbachol (producing a final volume of 50 μl), the FLIPR continued to collect data every 2 seconds for 240 seconds. The fluorescence emission was read using filter 1 (emission 520-545 nm) by the FLIPR on board CCD camera.

Calcium mobilization output data were calculated as the maximal relative fluorescence unit (RFU) minus the minal value for both compound and agonist reading frame (except for hM1 and rM1 using only the maximal RFU). Data were analyzed using sigmoidal fits of a non-linear curve-fitting program (XLfit version 5.0.6 from ID Business Solutions Limited, Guildford, UK). All EC50 and IC50 values are reported as arithmetic means±standard error of mean of ‘n’ independent experiments. Using the above-mentioned assays, the IC50 and EC50 towards human hM1, ratM1, hM3 and hM5 receptors for most compounds is measured to be in the range 1→30000 nM. The Eₘₐₓ (maximal effect, agonism or antagonist inhibition) towards human human hM1, ratM1, hM3 and hM5 receptors for most compounds Is measured to be in the range of 0-110%.

### hM2 Receptor GTPγS Binding

Membranes produced from Chinese hamster ovary cells (CHO) expressing the cloned human M2 receptor (human Muscarinic receptor subtype 2, gene bank access NM_{—}000739), were obtained from Perkin-Elmer (RBHM2M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 10012M DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM2 membranes, 500 μg of Flashblue beads (Perkin-Elmer) and GDP in a 25 μl volume. An additional 15 μl containing 3.3× (55000 dpm) of GTRγ35S (0.4 nM final) were added to the wells resulting in a total well volume of 50 μl. Basal and maximal stimulated [³⁵S]GTPγS binding were determined in absence and presence of 30 μM of acetylcholine agonist. The membranes/beads mix were pre-incubated for 15 minutes at room temperature with 25 μM GDP prior to distribution in plates (12.5 μM final). The reversal of acetylcholine-induced stimulation (2 μM final) of [³⁵S]GTPγS binding was used to assay the antagonist properties (IC₅₀) of the compounds. The plates were incubated for 60 minutes at room temperature with shaking, then centrifuged at 2000 rpm for 5 minutes. The radioactivity (cpm) were counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent stimulated [³⁵S]GTPγS binding vs. log(molar ligand).

All EC50 and IC50 values are reported as arithmetic means t standard error of mean of ‘n’ independent experiments. Based on the above assays, the EC₅₀ towards human M2 receptors for most compounds of the invention is measured to be in the range of about between 200 and >30000 nM. The Eₘₐₓ (maximal effect, agonism or antagonist inhibition) towards human M2 receptors for most compounds of the invention were measured to be in the range of about 0-120%. The IC₅₀ was the concentration of the compound of the invention at which 50% inhibition of acetylcholine [³⁵S]GTPγS binding stimulation has been observed. The IC₅₀ towards human M2 receptors for most compounds of the invention was measured to be in the range of between 40 and >90000 nM.

### HM4 Receptor GTPγS Binding

Membranes produced from Chinese hamster ovary cells (CHO) expressing the cloned human M4 receptor (human Muscarinic receptor subtype 4, gene bank access NM_{—}000741), were obtained from Perkin-Elmer (RBHM4M). The membranes were thawed at 37° C., passed 3 times through a 23-gauge blunt-end needle, diluted in the GTPγS binding buffer (50 mM Hepes, 20 mM NaOH, 100 mM NaCl, 1 mM EDTA, 5 mM MgCl₂, pH 7.4, 100 μM DTT). The EC₅₀, IC₅₀ and Eₘₐₓ of the compounds of the invention were evaluated from 10-point dose-response curves (three fold concentration range) done in 60 μl in 384-well non-specific binding surface plate (Corning). Ten microliters from the dose-response curves plate (5× concentration) were transferred to another 384 well plate containing the following: 10 μg of hM4 membranes, 500 μg of Flashblue beads (Perkin-Elmer) and GDP in a 25 μl volume. An additional 15 μl containing 3.3× (55000 dpm) of GTPγS (0.4 nM final) were added to the wells resulting in a total well volume of 50111. Basal and maximal stimulated [³⁵S]GTPγS binding were determined in absence and presence of 30 μM of acetylcholine agonist. The membranes/beads mix were pre-incubated for 15 minutes at room temperature with 40 μM GDP prior to distribution In plates (20 μM final). The reversal of acetylcholine-induced stimulation (10 μM final) of [³⁵S]GTPγS binding was used to assay the antagonist properties (IC₅₀) of the compounds. The plates were incubated for 60 minutes at room temperature with shaking, then centrifuged at 2000 rpm for 5 minutes. The radioactivity (cpm) were counted in a Trilux (Perkin-Elmer).

Values of EC₅₀, IC₅₀ and Eₘₐₓ were obtained using sigmoidal fits of a non-linear curve-fitting program (XLfit version 5.0.6 from ID Business Solutions Limited, Guildford, UK) of percent stimulated [³⁵S]GTRγS binding vs. log(molar ligand).

All EC50 and IC50 values are reported as arithmetic means±standard error of mean of ‘n’ independent experiments. Based on the above assays, the EC₅₀ towards human M4 receptors for most compounds of the invention is measured to be in the range of between 300 and >30000 nM. The Eₘₐₓ (maximal effect, agonism or antagonist inhibition) towards human M4 receptors for most compounds of the invention were measured to be in the range of about 0-120%. The IC₅₀ was the concentration of the compound of the invention at which 50% inhibition of acetylcholine [³⁵S]GTRγS binding stimulation has been observed. The IC₅₀ towards human M4 receptors for most compounds of the invention was measured to be in the range of between 3000 and >30000 nM.

Certain biological properties of certain compounds of the invention measured using one or more assays described above are listed in Table 1 below. The Example numbers of Table 1 correspond to the Example numbers of the Example section below.

TABLE 1

| | hM1 | hM2 | hM3 | hM4 | hM5 |
|---|---|---|---|---|---|
| | EC50 | EC50 | EC50 | EC50 | EC50 |
| Example No | (nM) | (nM) | (nM) | (nM) | (nM) |
| Example 10 | 286.3 | | | | |
| Example 11 | 611.8 | | | | |
| Example 12 | 2688.0 | | | | |
| Example 13 | 113.0 | 3542 | >40000 | >30000 | >40000 |
| Example 16 | 129.8 | 3287 | >40000 | >30000 | >40000 |
| Example 19 | 9.6 | 245 | 2077 | 577 | 833 |
| Example 21 | 1180.0 | | | | |
| Example 22 | 37.9 | >30000 | 49180 | >30000 | 49180 |
| Example 24 | 203.0 | >30000 | >40000 | >30000 | >40000 |
| Example 26 | 30.8 | 7449 | >40000 | >30000 | 341 |
| Example 29 | 3.5 | 1750 | 2500 | >30000 | 708 |
| Example 30 | 517.5 | | | | |
| Example 35 | 4.5 | >30000 | 5962 | >30000 | 905 |
| Example 36 | 40.8 | 4645 | >>40000 | >30000 | >40000 |
| Example 37 | 0.6 | | | | |
| Example 38 | 7.9 | | | | |
| Example 39 | 21.9 | | | | |
| Example 40 | 2.6 | >3333 | 622 | >10000 | 59 |
| Example 41 | 3.4 | >3333 | 2682 | >30000 | 534 |
| Example 42 | 3.0 | 1204 | | 10000 | |

### Rat FCA-Induced Heat Hyperalgesia Model (Prophetic)

Twenty four hours before testing, rats are brought to experimental lab. Rats are placed in a plexiglass chamber with 2% isoflurane at a flow rate of 0.8-1 L/hr with oxygen, for approximately 60-90 seconds, until a light-medium depth of anesthesia is attained. A volume of 25 μl of FCA is injected into the subcutaneous space of the dorsal aspect of the left hind paw, in the centre of the pads. This creates an inflammation, with accompanying edema and redness, as well as hyperalgesia, which is fully developed within 24 hours, and remains stable for weeks. In order to assess the degree of hyperalgesia, the animals are placed on a glass surface, and a heat-source is focused onto the plantar surface of the affected paw. The time from the initiation of the heat until the animal withdraws the paw is recorded. A decrease in Paw Withdrawal Latency (PWL) relative to naïve animals indicates a hyperalgesic state.

Generally, an experiment consists of 5 groups. One group is naïve and serves as baseline control. The other 4 groups receive FCA injection. One of the 4 groups serves as the vehicle control and the other receive drug treatment.

Drug or vehicle is administered 24 h after FCA inoculation. Rats are placed back in their home cage for 30 min, then, placed on the plantar apparatus for an additional 30 min for habituation. Total time of testing after drug administration is based on Tmax. The degree of reversal effect (heat hyperalgesia) is measured by the ability of a drug to return to normal levels (naïve PWL).

Statistical significance is determined using one-way ANOVA on raw data followed by a post-hoc Holm-Sidak t-test. The level of statistical significance is set at p≦0.05. Raw data are normalized using the following formula: % anti-hyperalgesia=═(PWL(dose)−PWL(vehicle))/(PWL(naïve)−PWL(vehicle))×100. Data is expressed as mean±SEM.

A combination containing a compound of the present invention and morphine at a predetermined ratio (e.g., 0.64:1) may be tested using this instant model. The combination drugs may be administered to the rats subcutaneously, orally or combination thereof, simultaneously or sequentially. The results (expressed as ED₅₀) for the combination may be compared with results obtained singly for the compound of the instant invention and morphine at the same or similar dosage range. If the ED₅₀ of the combination is significantly lower than the theoretical ED₅₀ calculated based on the ED₅₀ measured using the compound of the invention and morphine singly, then a synergy for the combination is indicated.

### EXAMPLES

The invention will further be described in more detail by the following Examples which describe methods whereby compounds of the present invention may be prepared, purified, analyzed and biologically tested, and which are not to be construed as limiting the invention.

### Example 1

### ethyl 3-[4-(2-oxo-2,3-dihydro-1H-indol-1-yl)piperidin-1-yl]pyrrolidine-1-carboxylate

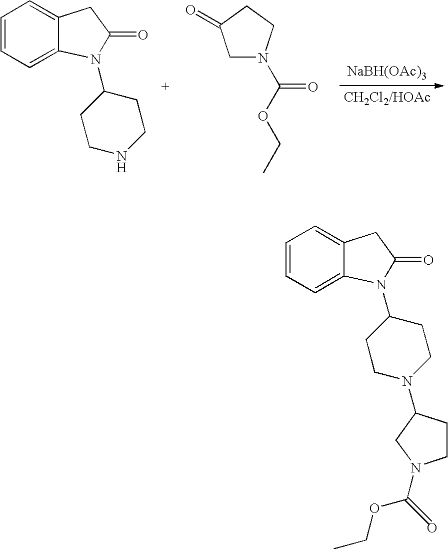

1-Piperindin-4-yl-1,3-dihydro-2H-indol-2-one (216.3 mg, 1 mmol), ethyl 3-oxopyrrolidine-1-carboxylate (157 mg, 1 mmol) and sodium triacetoxyborohydride (424 mg, 2 mmol) in CH₂Cl₂ (5 ml) and acetic acid (0.5 ml) were stirred at RT overnight. The reaction mixture was washed with 1M NaOH solution. The organic phase was collected and the aqueous phase was extracted with CH₂Cl₂ (2×). The combined organic phases was dried over MgSO₄, filtered, and concentrated in vacuo. The crude product was purified by flash chromatography, eluting with a gradient of 1:3 EtOAc/hexane to 1:2 EtOAc/hexane to give white solid (237 mg, 66% yield). The solid was re-purified by reverse phase HPLC (gradient 10-30% CH₃CN in H₂O containing 0.1% trifluoroacetic acid) to give white solid as TFA salt. ¹H NMR (400 MHz, METHANOL-D4): δ ppm 1.26 (t, J=7.13 Hz, 3H), 2.04 (d, J=17.58 Hz, 2H), 2.11-2.30 (m, 1H), 2.41-2.57 (m, 1H), 2.78-2.97 (m, 2H), 3.18-3.35 (m, 3H), 3.37-3.50 (m, 1H), 3.55 (s, 2H), 3.63-3.82 (m, 3H), 3.84-4.04 (m, 2H), 4.14 (q, J=7.10 Hz, 2H), 4.44 (t, J=12.01 Hz, 1H), 7.05 (t, J=7.52 Hz, 1H), 7.14 (d, J=7.81 Hz, 1H), 7.27 (t, J=8.30 Hz, 2H).

### Example 2

### Ethyl 3-[4-(2-oxo-2,3-dihydro-1H-benzimidazol-1-yl)piperidin-1-yl]pyrrolidine-1-carboxylate

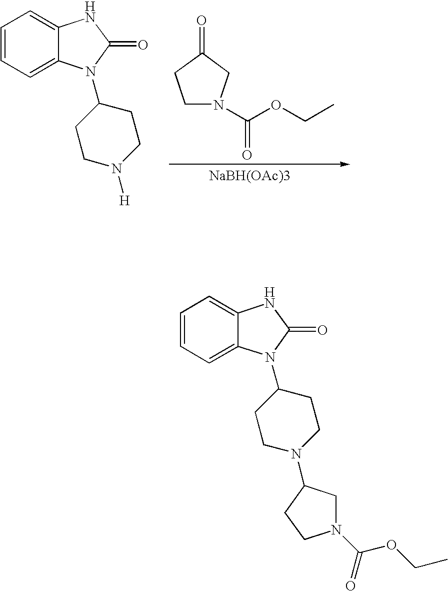

Following the procedure described in Example 1, the title compound was prepared from 1-piperidin-4-yl-1,3-dihydro-2H-benzimidazol-2-one and ethyl 3-oxopyrrolidine-1-carboxylate. ¹H NMR (400 MHz, CHLOROFORM-D): δ ppm 1.24 (t, J=7.03 Hz, 3H), 1.65-2.01 (m, 3H), 2.06-2.15 (m, 1H), 2.20 (q, J=6.90 Hz, 2H), 2.39-2.56 (m, 2H), 2.73-2.93 (m, 1H), 3.00 (d, J=10.16 Hz, 1H), 3.06-3.24 (m, 2H), 3.23-3.41 (m, 1H), 3.40-3.85 (m, 2H), 4.12 (q, J=6.90 Hz, 2H), 4.27-4.51 (m, 1H), 6.95-7.16 (m, 3H), 7.19-7.33 (m, 1H), 10.36 (s, 1H). MS: 359.3 (M+1).

### Example 3

### Ethyl 3-[4-(5-chloro-2-oxo-2,3-dihydro-1H-benzimidazol-1-yl)piperidin-1-yl]pyrrolidine-1-carboxylate

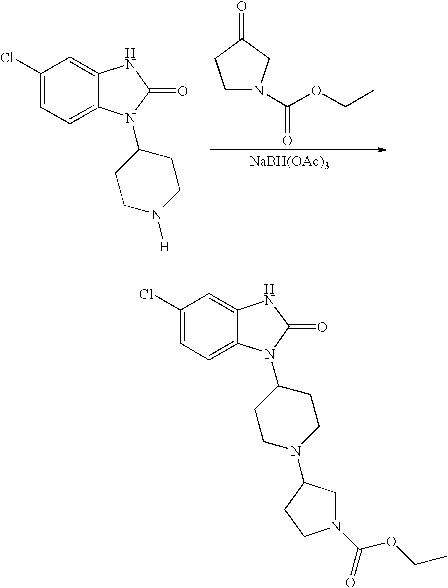

Following the procedure described in Example 1, the title compound was prepared from 5-chloro-1-(4-piperidyl)-2-benzimidazolinone hydrochloride (251.7 mg, 1 mmol)), ethyl 3-oxopyrrolidine-1-carboxylate (157 mg, 1 mmol). Ethyl 3-[4-(5-chloro-2-oxo-2,3-dihydro-1H-benzimidazol-1-yl)piperidin-1-yl]pyrrolidine-1-carboxylate was obtained as white solid. ¹H NMR (400 MHz, METHANOL-D4): δ ppm 1.24 (t, J=7.03 Hz, 3H), 1.97-2.14 (m, 2H), 2.17-2.35 (m, 1H), 2.41-2.59 (m, 1H), 2.69-2.94 (m, 2H), 3.19-3.51 (m, 3H), 3.56-3.83 (m, 4H), 3.85-4.03 (m, 2H), 4.11 (q, J=7.10 Hz, 2H), 4.47-4.68 (m, 1H), 7.00 (dd, J=8.40, 1.95 Hz, 1H), 7.05 (d, J=1.95 Hz, 1H), 7.24 (d, J=8.40 Hz, 1H).

### Example 4

### Benzyl 3-[4-(2-oxo-2,3-dihydro-1H-benzimidazol-1-yl)piperidin-1-yl]pyrrolidine-1-carboxylate

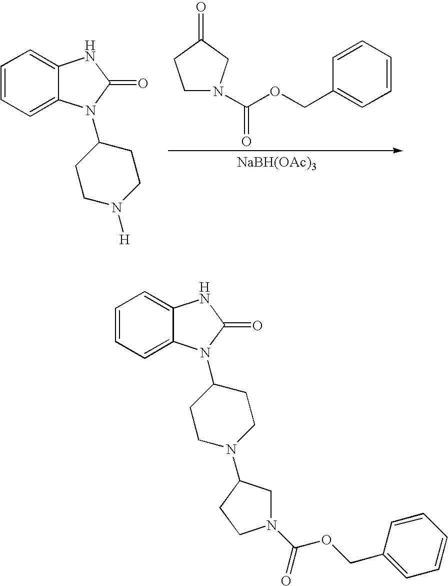

Following the procedure described in Example 1, the title compound was prepared from 1-piperidin-4-yl-1,3-dihydro-2H-benzimidazol-2-one and benzyl 3-oxopyrrolidine-1-carboxylate. ¹H NMR (400 MHz, CHLOROFORM-D): δ ppm 1.74-2.08 (m, 3H), 2.09-2.30 (m, 3H), 2.36-2.56 (m, 2H), 2.78-3.07 (m, 2H), 3.16 (d, J=10.94 Hz, 1H), 3.23 (q, J=9.50 Hz, 1H), 3.32-3.43 (m, 1H), 3.59-3.85 (m, 2H), 4.32-4.44 (m, 1H), 5.15 (s, 2H), 6.99-7.16 (m, 3H), 7.21-7.43 (m, 6H), 10.21 (s, 1H). MS (M+1): 420.95

### Example 5

### t-Butyl 3-[4-(2-oxo-2,3-dihydro-1H-benzimidazol-1-yl)piperidin-1-yl]pyrrolidine-1-carboxylate

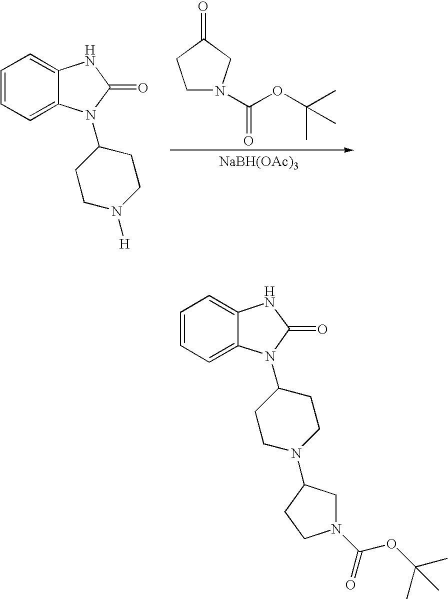

Following the procedure described in Example 1, the title compound was prepared from 1-piperidin-4-yl-1,3-dihydro-2H-benzimidazol-2-one and tert-butyl 3-oxopyrrolidine-1-carboxylate. ¹H NMR (400 MHz, CHLOROFORM-D): δ ppm 1.47 (s, 9H), 1.65 (s, 2H), 1.85 (s, 2H), 2.00-2.32 (m, 3H), 2.37-2.59 (m, 2H), 2.76-2.93 (m, 1H), 3.02 (d, J=12.30 Hz, 1H), 3.06-3.22 (m, 2H), 3.23-3.37 (m, 1H), 3.52 (3.63) (m, 1H), 3.72 (4.38) (m, 1H), 7.01-7.14 (m, 3H), 7.27 (s, 1H), 9.04 (s, 1H). MS (M+1): 386.97

### Example 6

### Isopropyl 3-[4-(2-oxo-2,3-dihydro-1H-benzimidazol-1-yl)piperidin-1-yl]pyrrolidine-1-carboxylate

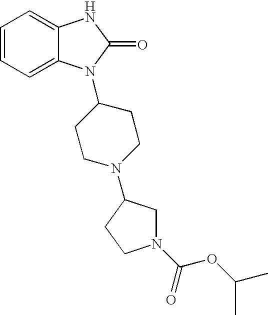

### Step A. The preparation of benzyl 3-{1-[1-(tert-butoxycarbonyl)pyrrolidin-3-yl]piperidin-4-yl}-2-oxo-2,3-dihydro-1H-benzimidazole-1-carboxylate

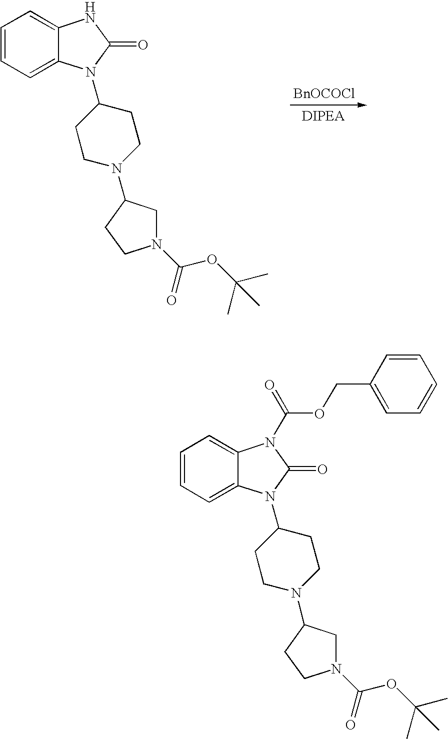

Benzyl chloroformate (450 μl, 3.15 mmol) was added to a solution of t-Butyl 3-[4-(2-oxo-2,3-dihydro-1H-benzimidazol-1-yl)piperidin-1-yl]pyrrolidine-1-carboxylate (1.11 g, 2.87 mmol), diisopropylethylamine (0.70 mL) in dichloromethane (15 mL) at room temperature and the mixture was stirred at room temperature overnight. Benzyl chloroformate (300 μl, 2.10 mmol) and diisopropylethylamine (0.30 mL) were added, and the mixture was stirred for another 4 h. Usual workup and purification on prep-HPLC (high pH) afforded the desired intermediate (780 mg). MS (M+1): 521.16.

### Step B. The preparation of benzyl 2-oxo-3-(1-pyrrolidin-3-ylpiperidin-4-yl)-2,3-dihydro-1H-benzimidazole-1-carboxylate

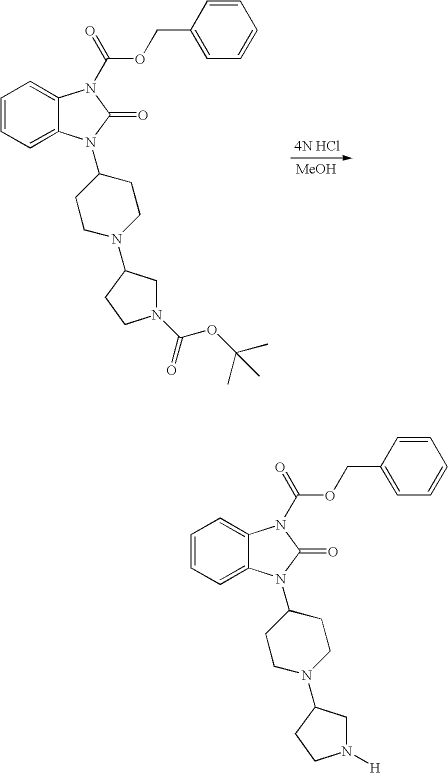

The intermediate (780 mg) from Step A was dissolved in methanol (30 mL) and 4 N HCl (6 mL, in dioxane) was added. The mixture was stirred at room temperature overnight. Removal of solvents provided the pyrrolidine intermediate as its HCl salt (730 mg). MS (M+1): 420.97.

Step C. The preparation of Isopropyl 3-[4-(2-oxo-2,3-dihydro-1H-benzimidazol-1-yl)piperidin-1-yl]pyrrolidine-1-carboxylate

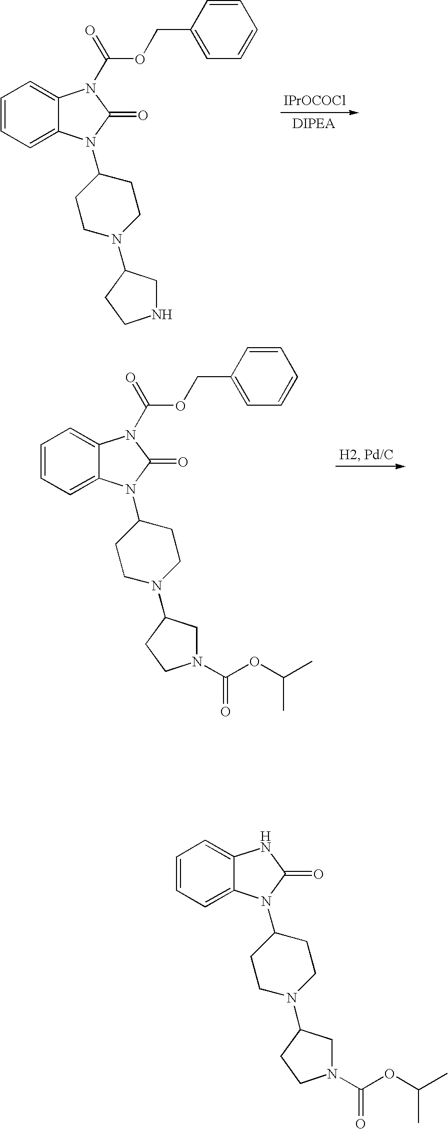

1 M isopropyl chloroformate (0.35 mL, 0.35 mmol, in toluene) was added to a solution of the pyrrolidine intermediate (134 mg, 0.273 mmol), and diisopropylethylamine (0.2 mL) in dichloromethane (8 mL). The mixture was stirred at room temperature, usual work afforded the intermediate (130 mg). MS (M+1): 506.98.

Hydrogenolysis of the above intermediate (130 mg) was performed in MeOH, 20 mg 10% Pd/C, H2 (25 psi), 4 N HCl in dioxane (1 mL) for 1 h. Removal of catalyst and solvent gave the crude product, which was purified on prep-HPLC (High pH). The free base was converted to HCl salt (73 mg). ¹H NMR (400 MHz, METHANOL-D4): δ ppm 1.25 (d, J=6.25 Hz, 6H), 2.08 (s, 2H), 2.25 (s, 1H), 2.48 (d, J=6.25 Hz, 1H), 2.75-3.04 (m, 2H), 3.29-3.50 (m, 3H), 3.49-3.86 (m, 5H), 3.95 (s, 2H), 4.42-4.69 (m, 1H), 6.80-7.19 (m, 3H), 7.43 (s, 1H). MS (M+1): 373.00

TABLE 1
Example 7-9 was prepared using similar method of Example 6

| Structure | | |
|---|---|---|
| (Example) | Name | NMR |
| 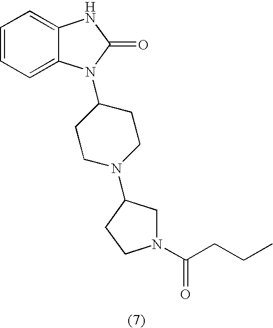 | 1-[1-(1-butyrylpyrrolidin-3- yl)piperidin-4-yl]-1,3- dihydro-2H-benzimidazol- 2-one | 1H NMR (400 MHz, METHANOL-D4) δ ppm 0.90-1.01 (m, 3H), 1.54-1.70 (m, 2H), 1.73-2.00 (m, 3H), 2.15-2.43 (m, 5H), 2.46-2.62 (m, 2H), 2.93-3.37 (m, 4H), 3.44-3.56 (m, 1H), 3.65-3.78 (m, 1H), 3.79-3.92 (m, 1H), 4.32 (t, J = 11.72 Hz, 1H), 6.99-7.09 (m, 3H), 7.29-7.37 (m, 1H) MS (M + 1): 357.3 |
| 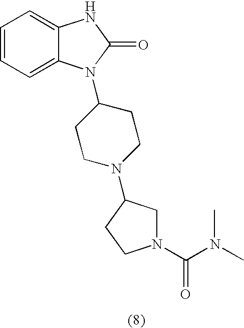 | N,N-dimethyl-3-[4-(2-oxo- 2,3-dihydro-1H- benzimidazol-1- yl)piperidin-1- yl]pyrrolidine-1- carboxamide | 1H NMR (400 MHz, METHANOL-D4) δ ppm 1.68-1.84 (m, 3H), 2.11-2.35 (m, 3H), 2.45-2.60 (m, 2H), 2.84 (s, 6H), 2.85-2.95 (m, 2H), 3.08 (d, J = 10.16 Hz, 1H), 3.22 (d, J = 11.72 Hz, 1H), 3.32 (d, J = 9.38 Hz, 1H), 3.40-3.53 (m, 2H), 3.56- 3.65 (m, 1H), 4.22-4.37 (m, 1H), 6.96 - 7.09 (m, 3H); 7.36 (dd, J = 7.03, 2.34 Hz, 1H) MS (M + 1): 358.3 |
| 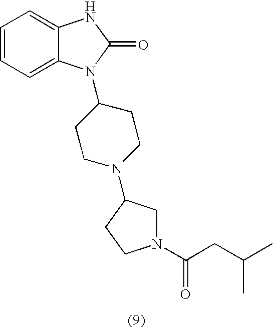 | 1-{1-[1-(3- methylbutanoyl)pyrrolidin- 3-yl]piperidin-4-yl}-1,3- dihydro-2H-benzimidazol- 2-one | 1H NMR (400 MHz, METHANOL-D4) δ ppm 0.90-0.99 (m, 6H), 1.73-1.94 (m, 3H), 1.95-2.16 (m, 1H), 2.22 (t, J = 7.81 Hz, 2H), 2.25-2.42 (m, 1H), 2.44-2.72 (m, 4H), 3.16-3.45 (m, 4H), 3.47-3.59 (m, 1H), 3.64-3.83 (m, 1H), 3.84-3.98 (m, 1H), 4.28-4.46 (m, 1H), 6.97-7.11 (m, 3H), 7.26-7.37 (m, 1H) MS (M + 1): 371.3 |

### Example 10

### Ethyl 3-[4-(3-methyl-2-oxo-2,3-dihydro-1H-benzimidazol-1-yl)piperidin-1-yl]pyrrolidine-1-carboxylate

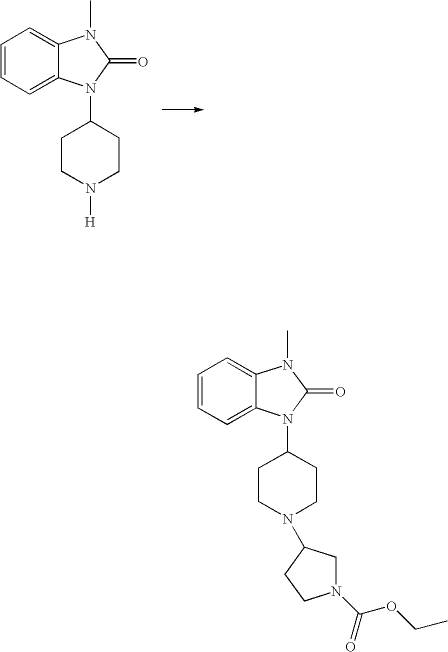

Following the similar procedure of Example 1, the title compound was prepared from 1-methyl-3-piperidin-4-yl-1,3-dihydro-2H-benzimidazol-2-one. ¹H NMR (400 MHz, CHLOROFORM-D): δ ppm 1.25 (t, J=7.03 Hz, 3H), 1.74-1.88 (m, 3H), 2.07-2.27 (m, 2H), 2.36-2.53 (m, 2H), 2.75-3.04 (m, 2H), 3.05-3.24 (m, 2H), 3.26-3.38 (m, 1H), 3.40 (s, 3H), 3.51-3.78 (m, 2H), 4.13 (q, J=7.03 Hz, 2H), 4.31-4.46 (m, 1H), 6.91-7.00 (m, 1H), 7.02-7.14 (m, 2H), 7.25-7.32 (m, 1H). MS: 373.3 (M+1).

### Example 11

### ethyl 3-[4-(1H-1,2,3-benzotriazol-1-yl)piperidin-1-yl]pyrrolidine-1-carboxylate

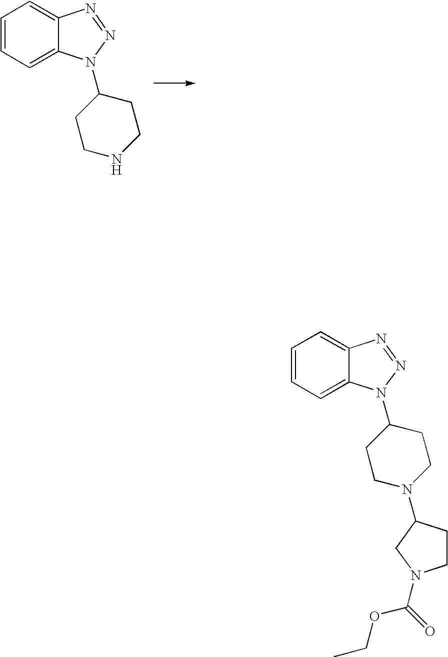

Following the similar procedure of Example 1, the title compound was prepared from 1-(4-piperidyl)-1H-1,2,3-benzotriazole hydrochloride (238.7 mg, 1 mmol)), 1N-ethoxycarbonyl-3-pyrrolidone (157 mg, 1 mmol). Ethyl 3-[4-(1H-1,2,3-benzotriazol-1-yl)piperidin-1-yl]pyrrolidine-1-carboxylate was obtained as white solid. ¹H NMR (400 MHz, METHANOL-D4): δ ppm 1.25 (t, J=7.13 Hz, 3H), 1.73-2.00 (m, 1H), 2.13-2.32 (m, 3H), 2.34-2.65 (m, 4H), 2.96-3.43 (m, 5H), 3.60 (t, J=10.94 Hz, 1H), 3.69-3.84 (m, 1H), 4.11 (q, J=7.03 Hz, 2H), 4.88-5.02 (m, 1H), 7.34-7.47 (m, 1H), 7.54 (t, J=7.71 Hz, 1H), 7.84 (d, J=8.40 Hz, 1H), 7.97 (d, J=8.40 Hz, 1H).

### Example 12

### Ethyl 3-[4-(2-oxo-1,2-dihydro-3H-indol-3-ylidene)piperidin-1-yl]pyrrolidine-1-carboxylate

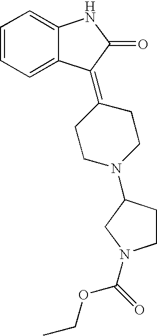

### Step A. The preparation of 3-piperidin-4-ylidene-1,3-dihydro-2H-indol-2-one

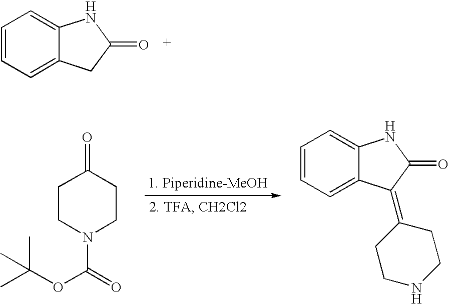

Oxindole (5 g, 37.6 mmol) and 1-Boc-4-piperidone (7.49 g, 37.6 mmol) in MeOH (100 ml) and piperidine (3.72 ml, 37.6 mmol) were heated at reflux for 3 hrs, allowed to cool to room temperature, and the yellow precipitate was collected. The filtrate was concentrated in vacuo to dryness, the residue was treated with MeOH (10 mL), and the solid was collected by filtration. The yellow solids were combined and dried (10 g, 85.3%).

The above solid intermediate (2 g) was dissolved in CH₂Cl₂ (100 mL), trifluoroacetic acid (6 mL) was added and the reaction was stirred for 2 hrs. CH₂Cl₂ was concentrated in vacuo to dryness. The colorless oil was obtained and used without purification.

### Step B. The preparation of ethyl 3-[4-(2-oxo-1,2-dihydro-3H-indol-3-ylidene)piperidin-1-yl]pyrrolidine-1-carboxylate

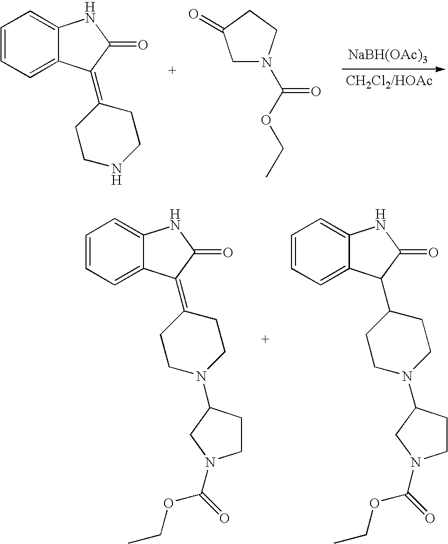

Following the similar procedure of Example 1, the title compound was prepared from 3-piperidin-4-ylidene-1,3-dihydro-2H-indol-2-one (136 mg, 0.637 mmol) and 1N-ethoxycarbonyl-3-pyrrolidone (100 mg, 0.637 mmol). Ethyl 3-[4-(5-chloro-2-oxo-2,3-dihydro-1H-benzimidazol-1-yl)piperidin-1-yl]pyrrolidine-1-carboxylate was obtained as yellow solid. ¹H NMR (400 MHz, METHANOL-D4): δ ppm 1.08-1.37 (m, 3H), 1.65-1.96 (m, 1H), 2.10-2.28 (m, 1H), 2.46-3.85 (m, 16H), 3.99-4.24 (m, 2H), 6.84 (d, J=7.23 Hz, 1H), 6.90-7.07 (m, 1H), 7.10-7.35 (m, 1H), 7.61 (d, J=7.81 Hz, 1H).

### Example 13

### Ethyl 3-[4-(2-oxo-2,3-dihydro-1H-indol-3-yl)piperidin-1-yl]pyrrolidine-1-carboxylate

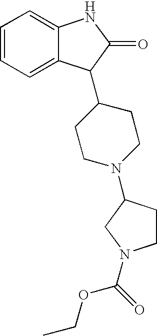

The title compound was prepared as by-product from Example 12, Step B. Ethyl 3-[4-(2-oxo-2,3-dihydro-1H-indol-3-yl)piperidin-1-yl]pyrrolidine-1-carboxylate was obtained as white solid. ¹H NMR (400 MHz, METHANOL-D4): δ ppm 1.16-1.27 (m, 3H), 1.70-2.16 (m, 5H), 2.31-2.52 (m, 2H), 2.95-3.17 (m, 2H), 3.33-3.46 (m, 1H), 3.48-3.68 (m, 4H), 3.75-3.94 (m, 2H), 4.11 (q, J=7.16 Hz, 2H), 5.47 (s, 1H), 6.88 (d, J=7.62 Hz, 1H), 6.99-7.05 (m, 1H), 7.22 (t, J=7.81 Hz, 1H), 7.29 (d, J=7.23 Hz, 1H).

### Example 14

### tert-Butyl (3S)-3-[4-(2-oxo-2,3-dihydro-1H-benzimidazol-1-yl)piperidin-1-yl]pyrrolidine-1-carboxylate

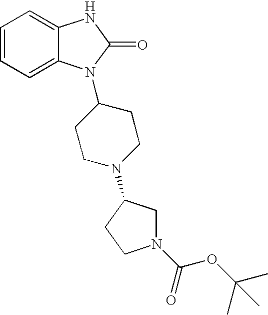

### Step A. The preparation of tert-butyl (3R)-3-[(methylsulfonyl)oxy]pyrrolidine-1-carboxylate

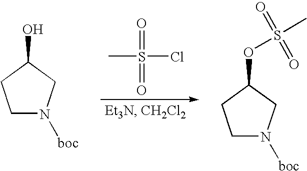

To (R)—N-Boc-3-pyrrolidinol (5 g, 26.7 mmol) in CH₂Cl₂(10 ml) at 0° C. was added Et₃N (4.12 g, 40.7 mmol), followed by methylsulfonyl chloride (3.81 g, 33.25 mmol) in 1 ml of CH₂Cl₂ slowly. The reaction mixture was warmed to RT and stirred overnight. The crude was washed with sat. NaHCO₃ solution (1×), extracted with CH₂Cl₂ (3×), and dried over MgSO₄. After filtration and evaporation, the residue was purified by chromatography on silica gel with 30% EtOAc/hexane to afford the mesylate tert-butyl (3R)-3-[(methylsulfonyl)oxy]pyrrolidine-1-carboxylate (4.26 g, 60.2%).

### Step B. The preparation of tert-butyl (3S)-3-[4-(2-oxo-2,3-dihydro-1H-benzimidazol-1-yl)piperidin-1-yl]pyrrolidine-1-carboxylate

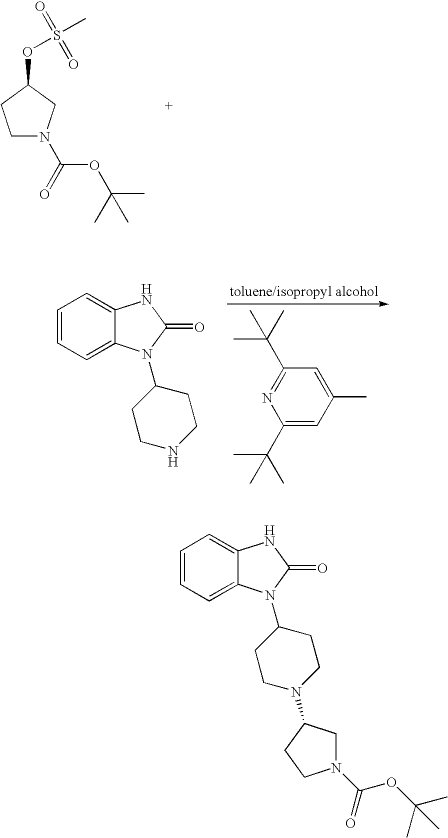

A mixture of tert-butyl (3R)-3-[(methylsulfonyl)oxy]pyrrolidine-1-carboxylate (462.5 mg, 1.74 mmol), 4-(2-keto-1-benzimidazolinyl)piperidine (250 mg, 1.15 mmol), 4-methyl-2,6-di-tert-butylpyridine (663 mg, 3.23 mmol) in 5 ml of toluene and 1 ml of isopropyl alcohol was heated at 100° C. overnight. The reaction mixture was partitioned between CH₂Cl₂/H₂O. The aqueous phase was further extracted with CH₂Cl₂(×2). The combined extracts were dried with MgSO₄, filtered and evaporated. The residue was purified by high pH HPLC to afford title compound (124 mg, 28%). tert-Butyl (3S)-3-[4-(2-oxo-2,3-dihydro-1H-benzimidazol-1-yl)piperidin-1-yl]pyrrolidine-1-carboxylate was obtained as white solid. ¹H NMR (400 MHz, METHANOL-D4): δ ppm 1.42-1.93 (m, 11H), 2.14-2.37 (m, 4H), 2.44-2.61 (m, 2H), 2.83-3.28 (m, 5H), 3.47-3.58 (m, 1H), 3.67 (t, J=10.74 Hz, 1H), 4.23-4.40 (m, 1H), 6.99-7.13 (m, 3H), 7.31-7.40 (m, 1H)

### Example 15

### Ethyl (3S)-3-[4-(2-oxo-2,3-dihydro-1H-benzimidazol-1-yl)piperidin-1-yl]pyrrolidine-1-carboxylate

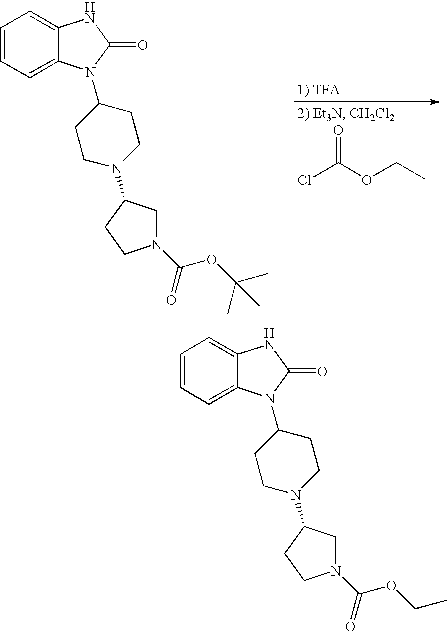

A mixture of tert-butyl (3S)-3-[4-(2-oxo-2,3-dihydro-1H-benzimidazol-1-yl)piperidin-1-yl]pyrrolidine-1-carboxylate (84 mg, 0.218 mmol) and trifluoroacetic acid (1 ml) in 2 ml of CH₂Cl₂ was stirred at RT for 2 hrs. The reaction mixture was evaporated to dryness and the crude was used without purification. To this amine in CH₂Cl₂ at −5° C. was added Et₃N (1 ml) followed by ethylchloroformate (21.7 mg, 0.2 mmol). The reaction mixture was stirred at ±5° C. for 10 mins, and then water was added to quench the reaction. The reaction mixture was partitioned between CH₂Cl₂/H₂O. The aqueous phase was further extracted with CH₂Cl₂(×2). The combined extracts were dried with MgSO₄, filtered and evaporated. The residue was purified by high pH HPLC to afford the title compound. Ethyl (3S)-3-[4-(2-oxo-2,3-dihydro-1H-benzimidazol-1-yl)piperidin-1-yl]pyrrolidine-1-carboxylate was obtained as white solid. Retention time=5.319 min, K′: 0.28 (Chiralpak OD column, 4.6×250 mm column 40% Ethanol/60% hexane, single peak). ¹H NMR (400 MHz, METHANOL-D4): δ ppm 1.25 (t, J=6.93 Hz, 3H), 1.70-1.93 (m, 3H), 2.14-2.35 (m, 3H), 2.44-2.60 (m, 2H), 2.88-3.10 (m, 2H), 3.12-3.24 (m, 2H), 3.26-3.40 (m, 1H), 3.52-3.64 (m, 1H), 3.67-3.79 (m, 1H), 4.11 (q, J=7.16 Hz, 2H), 4.24-4.38 (m, 1H), 6.99-7.10 (m, 3H), 7.31-7.39 (m, 1H)

### Example 16

### Ethyl (3R)-3-[4-(2-oxo-2,3-dihydro-1H-benzimidazol-1-yl)piperidin-1-yl]pyrrolidine-1-carboxylate

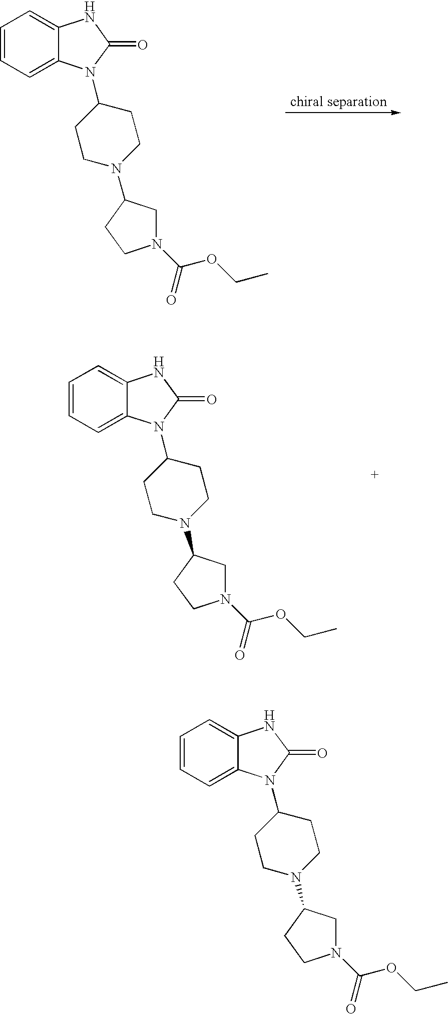

The solid from example 2 was resolved by chiral HPLC using OD column (gradient 10% EtOH in hexane containing 0.1% diethylamine) to give two enantiomers as white solid. The stereochemistry of title compound was determined by comparison of its retention time with compound from example 15. The first fraction was the title compound, and the second fraction was the same as Example 15. HPLC Retention time=5.021 min, K′: 0.21 (Chiralpak OD column, 4.6×250 mm column 40% Ethanol&Methanol/60% hexane, single peak). ¹H NMR (400 MHz, METHANOL-D4): δ ppm 1.25 (t, J=6.93 Hz, 3H), 1.70-1.93 (m, 3H), 2.14-2.35 (m, 3H), 2.44-2.60 (m, 2H), 2.88-3.10 (m, 2H), 3.12-3.24 (m, 2H), 3.26-3.40 (m, 1H), 3.52-3.64 (m, 1H), 3.67-3.79 (m, 1H), 4.11 (q, J=7.16 Hz, 2H), 4.24-4.38 (m, 1H), 6.99-7.10 (m, 3H), 7.31-7.39 (m, 1H)

### Example 17

### Methyl (3S)-3-[4-(2-oxo-2,3-dihydro-1H-benzimidazol-1-yl)piperidin-1-yl]pyrrolidine-1-carboxylate

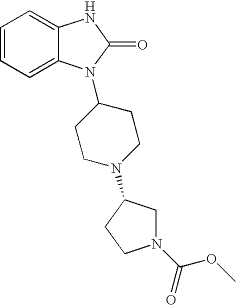

Following the procedure described in Example 15, the title compound was prepared from tert-butyl (3S)-3-[4-(2-oxo-2,3-dihydro-1H-benzimidazol-1-yl)piperidin-1-yl]pyrrolidine-1-carboxylate and methylchlorocarbamate. ¹H NMR (400 MHz, METHANOL-D4): δ ppm 1.64-1.93 (m, 3H), 2.11-2.35 (m, 3H), 2.42-2.61 (m, 2H), 2.85-3.08 (m, 2H), 3.11-3.23 (m, 2H), 3.24-3.40 (m, 1H), 3.52-3.61 (m, 1H), 3.63-3.77 (m, 4H), 4.17-4.40 (m, 1H), 6.91-7.12 (m, 3H), 7.20-7.42 (m, 1H). MS (M+1): 345.2

### Example 18

### Iso-Propyl (3S)-3-[4-(2-oxo-2,3-dihydro-1H-benzimidazol-1-yl)piperidin-1-yl]pyrrolidine-1-carboxylate

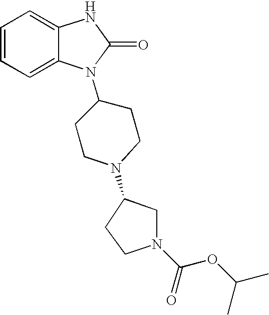

Following the procedure described in Example 15, the title compound was prepared from tert-butyl (3S)-3-[4-(2-oxo-2,3-dihydro-1H-benzimidazol-1-yl)piperidin-1-yl]pyrrolidine-1-carboxylate and isopropylchlorocarbamate (66% yield). ¹H NMR (400 MHz, METHANOL-D4): δ ppm 1.19-1.29 (m, 6H), 2.09 (s, 3H), 2.40-2.56 (m, 1H), 2.78-3.00 (m, 2H), 3.16-3.46 (m, 5H), 3.51-3.83 (m, 4H), 3.87-4.05 (m, 2H), 4.45-4.67 (m, 1H), 6.95-7.16 (m, 3H), 7.27-7.41 (m, 1H). MS (M+1): 373.3

### Example 19

### 1-{1-[(3S)-1-(cyclopentylcarbonyl)pyrrolidin-3-yl]piperidin-4-yl}-1,3-dihydro-2H-benzimidazol-2-one

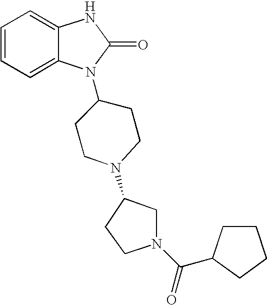

Following the procedure described in Example 15, the title compound was prepared from tert-butyl (3S)-3-[4-(2-oxo-2,3-dihydro-1H-benzimidazol-1-yl)piperidin-1-yl]pyrrolidine-1-carboxylate and cyclopentanecarbonylchloride (45% yield %). 1H NMR (400 MHz, METHANOL-04): δ ppm 1.46-1.88 (m, 11H), 2.13-2.32 (m, 3H), 2.38-2.52 (m, 2H), 2.80-2.91 (m, 1H), 2.92-3.05 (m, 1H), 3.09-3.32 (m, 4H), 3.42-3.92 (m, 2H), 4.13-4.32 (m, 1H), 6.92-7.00 (m, 3H), 7.24-7.31 (m, 1H). MS (M+1): 383.3

### Example 20

### 1-(1-{(3S)-1-[(2S)-tetrahydrofuran-2-ylcarbonyl]pyrrolidin-3-yl}piperidin-4-yl)-1,3-dihydro-2H-benzimidazol-2-one

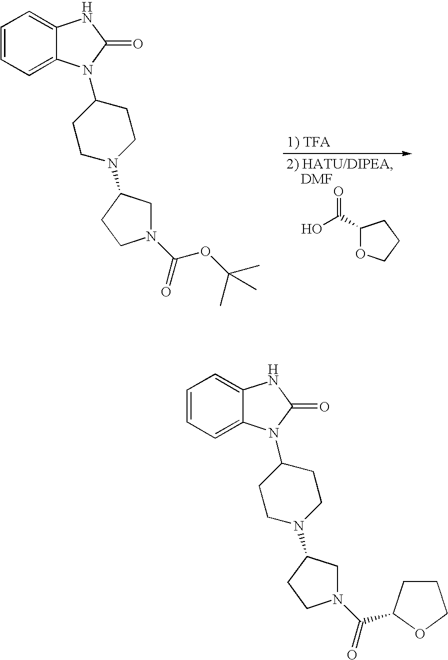

A mixture of tert-butyl (3S)-3-[4-(2-oxo-2,3-dihydro-1H-benzimidazol-1-yl)piperidin-1-yl]pyrrolidine-1-carboxylate (56.3 mg, 0.146 mmol) and trifluoroacetic acid (1 ml) in 2 ml of CH₂Cl₂ was stirred at RT for 2 hrs. The reaction mixture was evaporated to dryness and the crude was used without purification. To this amine and (2S)-tetrahydrofuran-2-carboxylic acid (17 mg, 0.146 mmol) in DMF at RT was added DIPEA (0.5 ml) followed by HATU (56 mg, 0.146 mmol). The mixture was stirred at RT for 2 h. The reaction was then concentrated in vacuo and the residue was diluted with brine. The aqueous phase was extracted with CH₂Cl₂ (3×). The combined organic phases was dried over MgSO₄, filtered, and concentrated in vacuo. The residue was purified by high pH HPLC to afford the title compound. 1-(1-{(3S)-1-[(2S)-tetrahydrofuran-2-ylcarbonyl]pyrrolidin-3-yl}piperidin-4-yl)-1,3-dihydro-2H-benzimidazol-2-one was obtained as white solid (12.7 mg, 23% yield). ¹H NMR (400 MHz, METHANOL-D4): δ ppm 1.35 (t, J=5.86 Hz, 1H), 1.81-2.70 (m, 7H), 2.80-3.08 (m, 2H), 3.13-4.12 (m, 11H), 4.20-4.42 (m, 1H), 4.54-4.73 (m, 2H), 7.01-7.15 (m, 3H), 7.44 (dd, J=24.41, 5.27 Hz, 1H). MS (M+1): 385.2

### Example 21

### 1-(1-{(3S)-1-[(1-methyl-1H-pyrrol-2-yl)carbonyl]pyrrolidin-3-yl}piperidin-4-yl)-1,3-dihydro-2H-benzimidazol-2-one

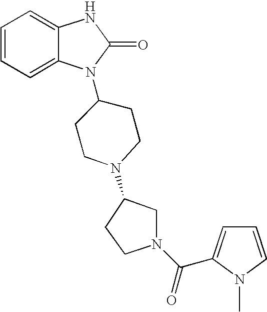

Following the procedure described in Example 20, the title compound was prepared from tert-butyl (3S)-3-[4-(2-oxo-2,3-dihydro-1H-benzimidazol-1-yl)piperidin-1-yl]pyrrolidine-1-carboxylate and 1-methyl-1H-pyrrole-2-carboxylic acid (95% yield). ¹H NMR (400 MHz, METHANOL-D4): δ ppm 1.97-2.16 (m, 2H), 2.20-2.37 (m, 1H), 2.47-2.64 (m, 1H), 2.79-3.00 (m, 2H), 3.17-3.46 (m, 2H), 3.64-4.06 (m, 9H), 4.09-4.32 (m, 1H), 4.52-4.68 (m, 1H), 6.03-6.15 (m, 1H), 6.64 (d, J=2.73 Hz, 1H), 6.85 (s, 1H), 6.97-7.20 (m, 3H), 7.35 (d, J=6.64 Hz, 1H). MS (M+1): 394.2

### Example 22

### 1-(1-{(3S)-1-[4-(2-oxopyrrolidin-1-yl)butanoyl]pyrrolidin-3-yl}piperidin-4-yl)-1,3-dihydro-2H-benzimidazol-2-one

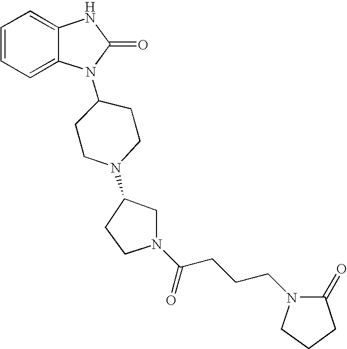

Following the procedure described in Example 20, the title compound was prepared from tert-butyl (3S)-3-[4-(2-oxo-2,3-dihydro-1H-benzimidazol-1-yl)piperidin-1-yl]pyrrolidine-1-carboxylate and 4-(2-oxopyrrolidin-1-yl)butanoic acid (49% yield). ¹H NMR (400 MHz, METHANOL-D4): δ ppm 1.68-1.96 (m, 5H), 1.95-2.08 (m, 2H), 2.13-2.40 (m, 7H), 2.43-2.60 (m, 2H), 2.83-3.38 (m, 7H), 3.41-3.54 (m, 3H), 3.63-3.74 (m, 1H), 3.77-3.87 (m, 1H), 4.20-4.38 (m, 1H), 6.96-7.13 (m, 3H), 7.28-7.42 (m, 1H). MS (M+1): 440.2

### Example 23

### 1-(1-{(3S)-1-[3-(2-oxopyrrolidin-1-yl)propanoyl]pyrrolidin-3-yl}piperidin-4-yl)-1,3-dihydro-2H-benzimidazol-2-one

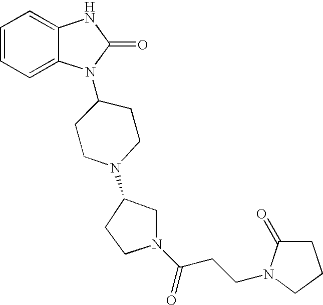

Following the procedure described in Example 20, 1-(1-{(3S)-1-[3-(2-oxopyrrolidin-1-yl)propanoyl]pyrrolidin-3-yl}piperidin-4-yl)-1,3-dihydro-2H-benzimidazol-2-one was prepared from tert-butyl (3S)-3-[4-(2-oxo-2,3-dihydro-1H-benzimidazol-1-yl)piperidin-1-yl]pyrrolidine-1-carboxylate and 3-(2-oxopyrrolidin-1-yl)propanoic acid (88% yield). ¹H NMR (400 MHz, METHANOL-D4): δ ppm 1.69-1.94 (m, 2H), 1.97-2.07 (m, 2H), 2.10-2.30 (m, 3H), 2.38 (t, J=8.20 Hz, 2H), 2.43-3.08 (m, 7H), 3.10-3.70 (m, 8H), 3.70-3.83 (m, 1H), 3.89 (dd, J=11.52, 7.23 Hz, 1H), 4.28-4.45 (m, 1H), 6.99-7.08 (m, 2H), 7.10-7.16 (m, 1H), 7.23-7.29 (m, 1H). MS (M+1): 426.2

### Example 24

### 1-methyl-3-(1-{(3S)-1-[3-(2-oxopyrrolidin-1-yl)propanoyl]pyrrolidin-3-yl}piperidin-4-yl)-1,3-dihydro-2H-benzimidazol-2-one

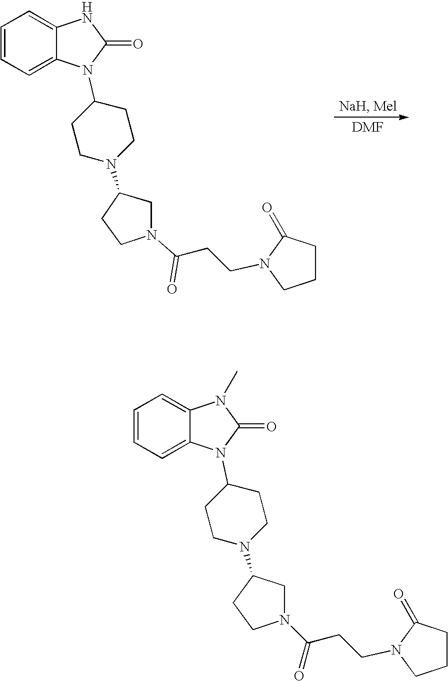

To 1-(1-{(3S)-1-[3-(2-oxopyrrolidin-1-yl)propanoyl]pyrrolidin-3-yl}piperidin-4-yl)-1,3-dihydro-2H-benzimidazol-2-one (63.5 mg, 0.149 mmol) in DMF (3 ml) at 0° C. was added NaH (18 mg, 0.745 mmol) and the reaction mixture was stirred at 0° C. for 0.5 hr. Methyl iodide (21 mg, 0.149 mmol) was added to this mixture at 0° C. and the reaction mixture was warmed to RT and stirred 2 hr. The reaction mixture was evaporated to dryness and the crude was washed with sat. NaHCO₃ solution (1×), extracted with CH₂Cl₂ (3×), and dried over MgSO₄. After filtration and evaporation, the residue was purified by high pH HPLC to afford 1-methyl-3-(1-{(3S)-1-[3-(2-oxopyrrolidin-1-yl)propanoyl]pyrrolidin-3-yl}piperidin-4-yl)-1,3-dihydro-2H-benzimidazol-2-one (47 mg, 71.7%). ¹H NMR (400 MHz, METHANOL-D4): δ ppm 1.68-2.10 (m, 5H), 2.14-2.38 (m, 5H), 2.43-2.69 (m, 4H), 2.83-3.11 (m, 2H), 3.12-3.24 (m, 1H), 3.24-3.35 (m, 3H), 3.37 (s, 3H), 3.44-3.61 (m, 3H), 3.62-3.98 (m, 2H), 4.21-4.44 (m, 1H), 7.01-7.17 (m, 3H), 7.36 (d, J=7.42 Hz, 1H). MS (M+1): 440.2

### Example 25

### (3S)—N-ethyl-3-[4-(2-oxo-2,3-dihydro-1H-benzimidazol-1-yl)piperidin-1-yl]pyrrolidine-1-carboxamide

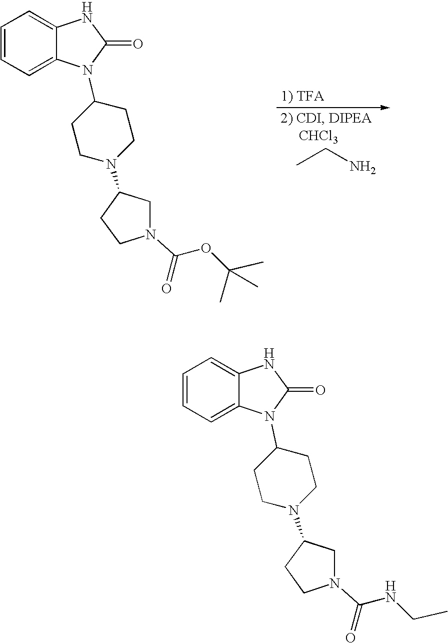

A mixture of tert-butyl (3S)-3-[4-(2-oxo-2,3-dihydro-1H-benzimidazol-1-yl)piperidin-1-yl]pyrrolidine-1-carboxylate (86 mg, 0.223 mmol) and trifluoroacetic acid (1 ml) in 2 ml of CH₂Cl₂ was stirred at RT for 2 hrs. The reaction mixture was evaporated to dryness and the crude was used without purification. A mixture of CDI (18 mg, 0.111 mmol), ethylamine (0.111 mmol) and DIPEA (1 ml) in CHCl₃ was stirred at RT for 15 mins. To this reaction mixture was added the amine prepared above and stirred at RT for 12 h. The reaction was diluted with brine and the aqueous phase was extracted with CH₂Cl₂ (3×). The combined organic phases was dried over MgSO₄, filtered, and concentrated in vacuo. The residue was purified by high pH HPLC to afford the title compound. (3S)—N-ethyl-3-[4-(2-oxo-2,3-dihydro-1H-benzimidazol-1-yl)piperidin-1-yl]pyrrolidine-1-carboxamide was obtained as white solid (20.6 mg, 26% yield). ¹H NMR (400 MHz, METHANOL-D4): δ ppm 1.12-1.19 (m, 3H), 1.73-2.00 (m, 5H), 2.12-2.34 (m, 3H), 2.40-2.58 (m, 2H), 2.84-2.97 (m, 1H), 3.02 (d, J=12.11 Hz, 1H), 3.16 (t, J=8.79 Hz, 2H), 3.23-3.39 (m, 3H), 3.56 (t, J=8.98 Hz, 1H), 3.71 (t, J=8.20 Hz, 1H), 4.16 (t, J=5.47 Hz, 1H), 4.28-4.50 (m, 1H), 6.95-7.16 (m, 3H), 7.23-7.40 (m, 1H). MS (M+1): 358.3

### Example 26

### Ethyl (3S)-3-[4-(2-oxo-2,3-dihydro-1H-indol-1-yl)piperidin-1-yl]pyrrolidine-1-carboxylate

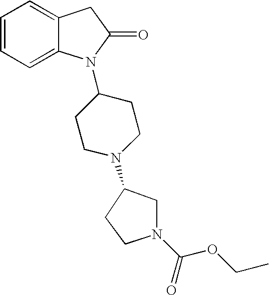

### Step A. The preparation of tert-butyl (3S)-3-[4-(2-oxo-2,3-dihydro-1H-indol-1-yl)piperidin-1-yl]pyrrolidine-1-carboxylate

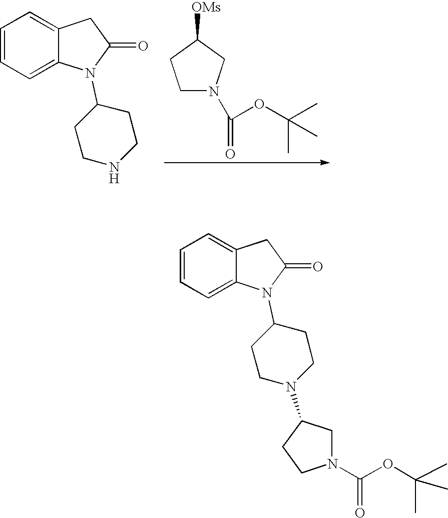

Following the procedure described in Example 14, Step B, tert-butyl (3S)-3-[4-(2-oxo-2,3-dihydro-1H-indol-1-yl)piperidin-1-yl]pyrrolidine-1-carboxylate (24% yield) was prepared from 1-piperidin-4-yl-1,3-dihydro-2H-indol-2-one, tert-butyl (3R)-3-[(methylsulfonyl)oxy]pyrrolidine-1-carboxylate.

### Step B: The preparation of Ethyl (3S)-3-[4-(2-oxo-2,3-dihydro-1H-indol-1-yl)piperidin-1-yl]pyrrolidine-1-carboxylate

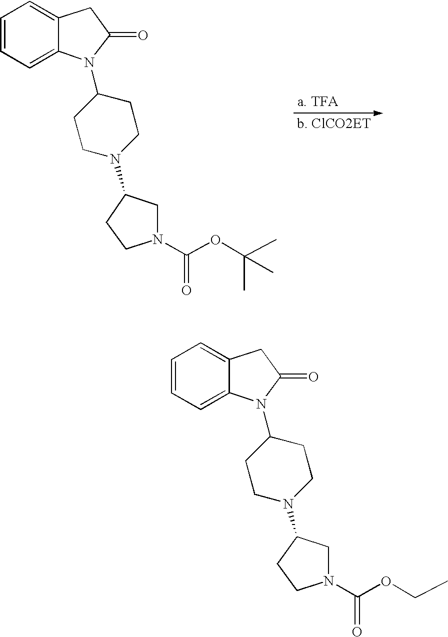

Following the similar procedure described in Example 15, the title compound was tert-butyl (3S)-3-[4-(2-oxo-2,3-dihydro-1H-indol-1-yl)piperidin-1-yl]pyrrolidine-1-carboxylate and ethyl chloroformate (57% yield). ¹H NMR (400 MHz, METHANOL-D4): δ ppm 1.26 (t, J=7.13 Hz, 3H), 2.04 (d, J=17.58 Hz, 2H), 2.11-2.30 (m, 1H), 2.41-2.57 (m, 1H), 2.78-2.97 (m, 2H), 3.18-3.35 (m, 3H), 3.37-3.50 (m, 1H), 3.55 (s, 2H), 3.63-3.82 (m, 3H), 3.84-4.04 (m, 2H), 4.14 (q, J=7.10 Hz, 2H), 4.44 (t, J=12.01 Hz, 1H), 7.05 (t, J=7.52 Hz, 1H), 7.14 (d, J=7.81 Hz, 1H), 7.27 (t, J=8.30 Hz, 2H). MS (M+1): 358.1

### Example 27

### Ethyl (3R)-3-[4-(2-oxo-2,3-dihydro-1H-indol-1-yl)piperidin-1-yl]pyrrolidine-1-carboxylate

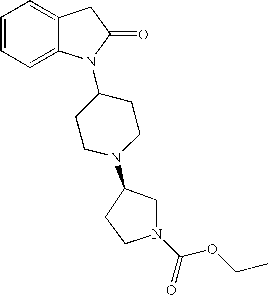

Following the procedure described in Example 16, the title compound was obtained from chiral separation of racemic mixture from example 1. The first fraction from chiral AD column (20% isopropanol/hexane) was collected as the title compound; ¹H NMR (400 MHz, METHANOL-D4): δ ppm 1.26 (t, J=7.13 Hz, 3H), 2.04 (d, J=17.58 Hz, 2H), 2.11-2.30 (m, 1H), 2.41-2.57 (m, 1H), 2.78-2.97 (m, 2H), 3.18-3.35 (m, 3H), 3.37-3.50 (m, 1H), 3.55 (s, 2H), 3.63-3.82 (m, 3H), 3.84-4.04 (m, 2H), 4.14 (q, J=7.10 Hz, 2H), 4.44 (t, J=12.01 Hz, 1H), 7.05 (t, J=7.52 Hz, 1H), 7.14 (d, J=7.81 Hz, 1H), 7.27 (t, J=8.30 Hz, 2H). MS (M+1): 358.1

### Example 28

### Methyl (3S)-3-[4-(2-oxo-2,3-dihydro-1H-indol-1-yl)piperidin-1-yl]pyrrolidine-1-carboxylate

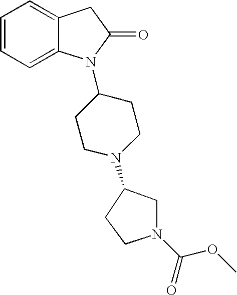

Following the procedure described in Example 26, the title compound was prepared from tart-butyl (3S)-3-[4-(2-oxo-2,3-dihydro-1H-indol-1-yl)piperidin-1-yl]pyrrolidine-1-carboxylate and methyl chloroformate (66% yield). ¹H NMR (400 MHz, METHANOL-D4): δ ppm 2.01 (d, J=12.89 Hz, 2H), 2.21-2.36 (m, 1H), 2.41-2.58 (m, 1H), 2.83-3.05 (m, 2H), 3.22-3.57 (m, 4H), 3.59-3.82 (m, 7H), 3.94 (s, 2H), 4.52 (t, J=12.30 Hz, 1H), 7.02 (t, J=7.42 Hz, 1H), 7.20-7.40 (m, 3H). MS (M+1): 344.3

### Example 29

### 1-(1-{(3S)-1-[3-(2-oxopyrrolidin-1-yl)propanoyl]pyrrolidin-3-yl}piperidin-4-yl)-1,3-dihydro-2H-indol-2-one

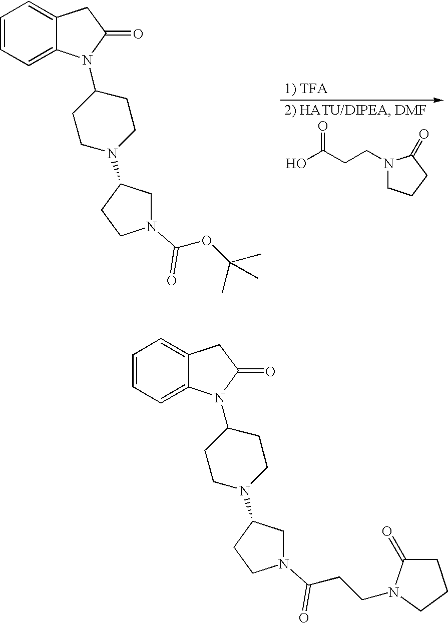

A mixture of tert-butyl (3S)-3-[4-(2-oxo-2,3-dihydro-1H-indol-1-yl)piperidin-1-yl]pyrrolidine-1-carboxylate (80 mg, 0.208 mmol) and trifluoroacetic acid (1 ml) in 2 ml of CH₂Cl₂ was stirred at RT for 2 hrs. The reaction mixture was evaporated to dryness and the crude was used without purification. To this amine and 3-(2-oxopyrrolidin-1-yl)propanoic acid (33 mg, 0.208 mmol) in DMF at RT was added DIPEA (1 ml) followed by HATU (80 mg, 0.208 mmol). The mixture was stirred at RT for 2 h. The reaction was then concentrated in vacuo and the residue was diluted with brine. The aqueous phase was extracted with CH₂Cl₂ (3×), combined organic phases was dried over MgSO₄, filtered, and concentrated in vacuo. The residue was purified by high pH HPLC to afford the title compound. 1-(1-{(3S)-1-[3-(2-oxopyrrolidin-1-yl)propanoyl]pyrrolidin-3-yl}piperidin-4-yl)-1,3-dihydro-2H-indol-2-one was obtained as white solid (42.8 mg, 49% yield). ¹H NMR (400 MHz, METHANOL-D4): δ ppm 1.92-2.10 (m, 4H), 2.16-2.75 (m, 6H), 2.84-3.07 (m, 2H), 3.23-3.46 (m, 4H), 3.47-4.24 (m, 10H), 4.44-4.66 (m, 1H), 7.02 (t, J=7.42 Hz, 1H), 7.20-7.47 (m, 3H). MS (M+1): 425.2

### Example 30

### Ethyl 3-[3-(2-oxo-2,3-dihydro-1H-benzimidazol-1-yl)-8-azabicyclo[3.2.1]oct-8-yl]pyrrolidine-1-carboxylate

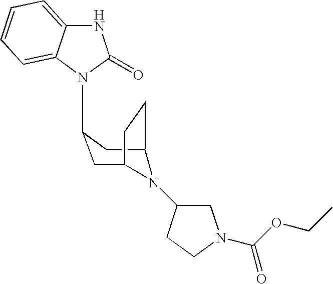

### Step A. The preparation of tert-butyl 3-[(2-aminophenyl)amino]-8-azabicyclo[3.2.1]octane-8-carboxylate

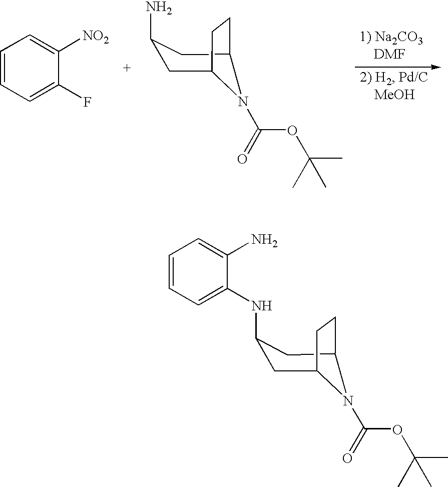

tert-Butyl 3-amino-8-azabicyclo[3.2.1]octane-8-carboxylate (480 mg, 2.124 mmol), 2-fluoronitrobenzene (300 mg, 2.124 mmol) and Na₂CO₃ (674 mg, 6.36 mmol) in DMF (20 ml) was heated at 100° C. for 2 hrs. DMF was evaporated and the crude was washed with brine, extracted with CH₂Cl₂ (3×) and dried over MgSO₄. After filtration, solvent was removed by evaporation and the residue was obtained as orange oil and used without purification.

A solution of this orange oil prepared above in methanol was stirred in the presence of Palladium on Charcoal (50 mg) under a hydrogen atmosphere for 8 hrs. The reaction mixture was filtered over Celite and concentrated to give tert-butyl 3-[(2-aminophenyl)amino]-8-azabicyclo[3.2.1]octane-8-carboxylate.

### Step B. The preparation of tert-butyl 3-(2-oxo-2,3-dihydro-1H-benzimidazol-1-yl)-8-azabicyclo[3.2.1]octane-8-carboxylate

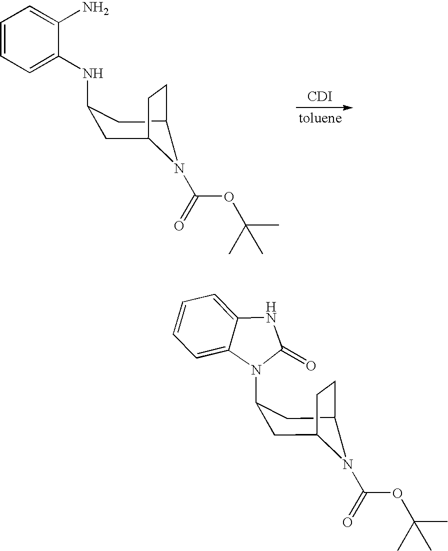

A mixture of tert-butyl 3-[(2-aminophenyl)amino]-8-azabicyclo[3.2.1]octane-8-carboxylate (337 mg, 1.062 mmol) and CDI (517 mg, 3.186 mmol) in toluene was heated at refluxed for 24 hrs. Toluene was evaporated and the residue was washed with brine, extracted with CH₂Cl₂ (3×) and dried over MgSO₄. After filtration and evaporation, the residue was purified by high pH HPLC to afford the title compound.

tert-Butyl 3-(2-oxo-2,3-dihydro-1H-benzimidazol-1-yl)-8-azabicyclo[3.2.1]octane-8-carboxylate was obtained as pale pink solid (176.6 mg, 48.5% yield).

### Step C. The preparation of ethyl 3-[3-(2-oxo-2,3-dihydro-1H-benzimidazol-1-yl)-8-azabicyclo[3.2.1]oct-8-yl]pyrrolidine-1-carboxylate

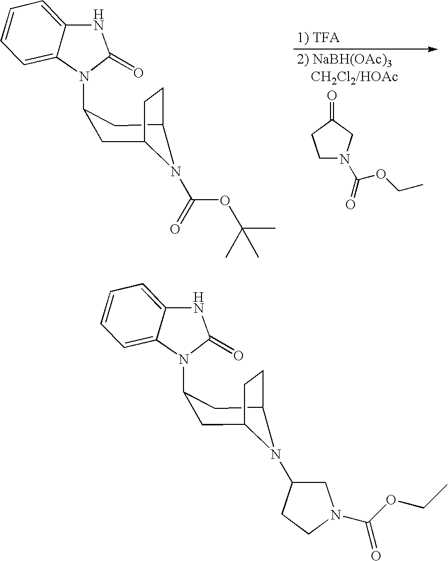

### tert-Butyl 3-(2-oxo-2,3-dihydro-1H-benzimidazol-1-yl)-8-azabicyclo[3.2.1]octane-8-carboxylate (176.6 mg, 0.515 mmol) and trifluoroacetic acid (1 ml) in CH₂Cl₂ (5 ml) was stirred at RT for 2 hrs. The reaction mixture was evaporated to dryness and the crude was used without purification. This amine, ethyl 3-oxopyrrolidine-1-carboxylate (81 mg, 0.515 mmol) and sodium triacetoxyborohydride (327 mg, 1.545 mmol) in CH₂Cl₂ (5 ml) and acetic acid (0.5 ml) were stirred at RT overnight. The reaction mixture was washed with 1M NaOH., organic phase was collected and the aqueous phase was extracted with CH₂Cl₂ (2×). The combined organic phases was dried over MgSO₄, filtered, and concentrated in vacuo. The crude product was purified by high pH HPLC to afford the title compound. Ethyl 3-[3-(2-oxo-2,3-dihydro-1H-benzimidazol-1-yl)-8-azabicyclo[3.2.1]oct-8-yl]pyrrolidine-1-carboxylate was obtained as white solid (140.3 mg, 71% yield). ¹H NMR (400 MHz, METHANOL-D4): δ ppm 1.16-1.31 (m, 3H), 1.64-1.85 (m, 3H), 1.93 (t, J=12.21 Hz, 2H), 2.00-2.22 (m, 3H), 2.24-2.40 (m, 2H), 2.84-2.99 (m, 1H), 3.11 (q, J=8.92 Hz, 1H), 3.21-3.43 (m, 3H), 3.47-3.69 (m, 3H), 4.09 (q, J=7.03 Hz, 2H), 4.59-4.78 (m, 1H), 6.91-7.19 (m, 4H). MS (M+1): 385.3

### Example 31

### Ethyl 3-[4-(7-fluoro-2-oxo-2,3-dihydro-1H-benzimidazol-1-yl)piperidin-1-yl]pyrrolidine-1-carboxylate

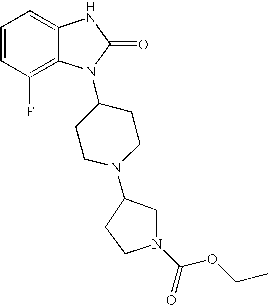

Following the procedure described in Example 30, the title compound was prepared from tert-butyl 4-aminopiperidine-1-carboxylate, 2,3-difluoronitrobenzene and ethyl 3-oxopyrrolidine-1-carboxylate. ¹H NMR (400 MHz, METHANOL-D4): δ ppm 1.14-1.31 (m, 3H), 1.81-2.06 (m, 3H), 2.47-2.69 (m, 2H), 3.18-3.48 (m, 8H), 3.54-3.69 (m, 1H), 3.73-3.88 (m, 1H), 4.11 (q, J=7.03 Hz, 2H), 4.46-4.67 (m, 1H), 6.82-6.92 (m, 2H), 6.98-7.09 (m, 1H). MS (M+1): 377.3

### Example 32

### Ethyl 3-[4-(5-fluoro-2-oxo-2,3-dihydro-1H-benzimidazol-1-yl)piperidin-1-yl]pyrrolidine-1-carboxylate

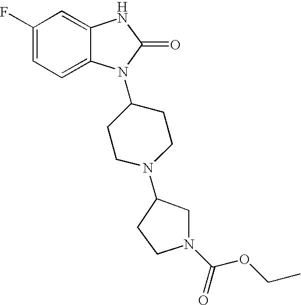

Following the procedure described in Example 30, the title compound was prepared from tert-butyl 4-aminopiperidine-1-carboxylate, 2,5-difluoronitrobenzene and ethyl 3-oxopyrrolidine-1-carboxylate. ¹H NMR (400 MHz, METHANOL-D4): δ ppm 1.25 (t, J=7.13 Hz, 3H), 2.03-2.30 (m, 3H), 2.40-2.56 (m, 1H), 2.70-2.90 (m, 2H), 3.18-3.35 (m, 2H), 3.38-3.60 (m, 2H), 3.62-3.82 (m, 3H), 3.87-4.03 (m, 2H), 4.13 (q, J=7.10 Hz, 2H), 4.42-4.62 (m, 1H), 6.77-6.88 (m, 2H), 7.19 (dd, J=8.69, 4.20 Hz, 1H). MS (M+1): 377.3

### Example 33

### Ethyl 3-[4-(4-fluoro-2-oxo-2,3-dihydro-1H-benzimidazol-1-yl)piperidin-1-yl]pyrrolidine-1-carboxylate

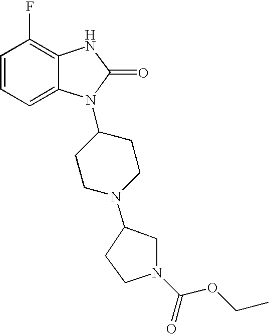

Following the procedure described in Example 30, the title compound was prepared from tert-butyl 4-aminopiperidine-1-carboxylate, 2,6-difluoronitrobenzene and ethyl 3-oxopyrrolidine-1-carboxylate. ¹H NMR (400 MHz, METHANOL-D4): δ ppm 1.25 (t, J=7.13 Hz, 3H), 2.10 (d, J=16.21 Hz, 2H), 2.38-2.59 (m, 1H), 2.72-2.94 (m, 2H), 3.18-3.36 (m, 3H), 3.38-3.82 (m, 5H), 3.87-4.02 (m, 2H), 4.13 (q, J=6.97 Hz, 2H), 4.46-4.65 (m, 1H), 6.82-6.92 (m, 1H), 6.97-7.16 (m, 2H). MS (M+1): 377.3

### Example 34

### Ethyl 3-[4-(6-fluoro-2-oxo-2,3-dihydro-1H-benzimidazol-1-yl)piperidin-1-yl]pyrrolidine-1-carboxylate

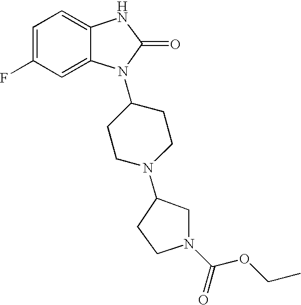

Following the procedure described in Example 30, the title compound was prepared from tert-butyl 4-aminopiperidine-1-carboxylate, 2,4-difluoronitrobenzene and ethyl 3-oxopyrrolidine-1-carboxylate. ¹H NMR (400 MHz, METHANOL-D4): δ ppm 1.24 (t, J=7.13 Hz, 3H), 1.74-2.03 (m, 3H), 2.29 (t, J=12.89 Hz, 1H), 2.40-2.69 (m, 4H), 3.09-3.44 (m, 5H), 3.53-3.66 (m, 1H), 3.71-3.87 (m, 1H), 4.11 (q, J=7.16 Hz, 2H), 4.26-4.43 (m, 1H), 6.69-6.85 (m, 1H), 6.99 (dd, J=8.59, 4.49 Hz, 1H), 7.21 (dd, J=9.37, 2.34 Hz, 1H). MS (M+1): 377.3

### Example 35 and Example 36

### (3S) Ethyl 3-[4-(6-fluoro-2-oxo-2,3-dihydro-1H-benzimidazol-1-yl)piperidin-1-yl]pyrrolidine-1-carboxylate and (3R) Ethyl 3-[4-(6-fluoro-2-oxo-2,3-dihydro-1H-benzimidazol-1-yl)piperidin-1-yl]pyrrolidine-1-carboxylate

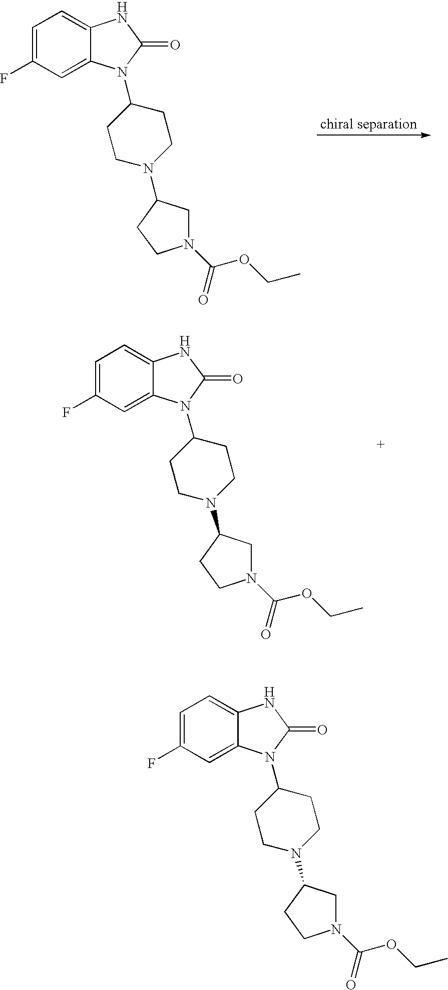

The solid from example 34 was separated by chiral HPLC (10% isopropanol in hexane, Chiral OD column) to give two enantiomers as white solid. Isomer 1 (Example 36). HPLC Retention time=14.19 min (15% isopropanol in hexane, chiralpack OD column, 4.6×250 mm column). ¹H NMR (400 MHz, METHANOL-D4): δ ppm 1.25 (t, J=6.93 Hz, 3H), 1.70-1.93 (m, 3H), 2.14-2.35 (m, 3H), 2.44-2.60 (m, 2H), 2.88-3.10 (m, 2H), 3.12-3.24 (m, 2H), 3.26-3.40 (m, 1H), 3.52-3.64 (m, 1H), 3.67-3.79 (m, 1H), 4.11 (q, J=7.16 Hz, 2H), 4.24-4.38 (m, 1H), 6.99-7.10 (m, 3H), 7.31-7.39 (m, 1H).

Isomer 2 (Example 35): HPLC Retention time=16.50 min (15% isopropanol in hexane, chiralpack OD column, 4.6×250 mm column). ¹H NMR (400 MHz, METHANOL-D4): δ ppm 1.25 (t, J=6.93 Hz, 3H), 1.70-1.93 (m, 3H), 2.14-2.35 (m, 3H), 2.44-2.60 (m, 2H), 2.88-3.10 (m, 2H), 3.12-3.24 (m, 2H), 3.26-3.40 (m, 1H), 3.52-3.64 (m, 1H), 3.67-3.79 (m, 1H), 4.11 (q, J=7.16 Hz, 2H), 4.24-4.38 (m, 1H), 6.99-7.10 (m, 3H), 7.31-7.39 (m, 1H).

### Example 37 and Example 38

### (3S) Ethyl 3-[4-(6-methyl-2-oxo-2,3-dihydro-1H-benzimidazol-1-yl)piperidin-1-yl]pyrrolidine-1-carboxylate and (3R) ethyl 3-[4-(6-methyl-2-oxo-2,3-dihydro-1H-benzimidazol-1-yl)piperidin-1-yl]pyrrolidine-1-carboxylate

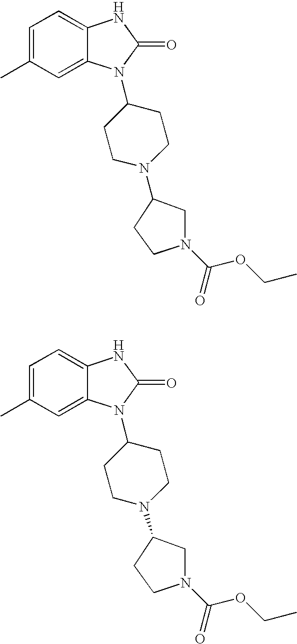

Following the procedure described in Example 30 and 35, the title compounds were prepared from tert-butyl 4-aminopiperidine-1-carboxylate, 4-methyl-2-fluoronitrobenzene and ethyl 3-oxopyrrolidine-1-carboxylate.

Isomer 1 (Example 38). ¹H NMR (400 MHz, METHANOL-D4): δ ppm 1.23 (t, J=7.03 Hz, 3H), 1.63-1.87 (m, 3H), 2.08-2.27 (m, 3H), 2.34 (s, 3H), 2.40-2.56 (m, 2H), 2.79-3.02 (m, 2H), 3.13 (t, J=9.57 Hz, 2H), 3.21-3.36 (m, 1H), 3.55 (t, J=9.18 Hz, 1H), 3.62-3.74 (m, 1H), 4.09 (q, J=7.03 Hz, 2H), 4.20-4.34 (m, 1H), 6.80-6.85 (m, 1H), 6.88-6.95 (m, 1H), 7.20 (s, 1H). MS (M+1): 373.3.

Isomer 2 (Example 37). ¹H NMR (400 MHz, METHANOL-D4): δ ppm 1.23 (t, J=7.03 Hz, 3H), 1.63-1.87 (m, 3H), 2.08-2.27 (m, 3H), 2.34 (s, 3H), 2.40-2.56 (m, 2H), 2.79-3.02 (m, 2H), 3.13 (t, J=9.57 Hz, 2H), 3.21-3.36 (m, 1H), 3.55 (t, J=9.18 Hz, 1H), 3.62-3.74 (m, 1H), 4.09 (q, J=7.03 Hz, 2H), 4.20-4.34 (m, 1H), 6.80-6.85 (m, 1H), 6.88-6.95 (m, 1H), 7.20 (s, 1H). MS (M+1): 373.3

### Example 39 and Example 40

### (3S) Ethyl 3-[4-(6-methoxy-2-oxo-2,3-dihydro-1H-benzimidazol-1-yl)piperidin-1-yl]pyrrolidine-1-carboxylate and (3R) ethyl 3-[4-(6-methoxy-2-oxo-2,3-dihydro-1,1-benzimidazol-1-yl)piperidin-1-yl]pyrrolidine-1-carboxylate

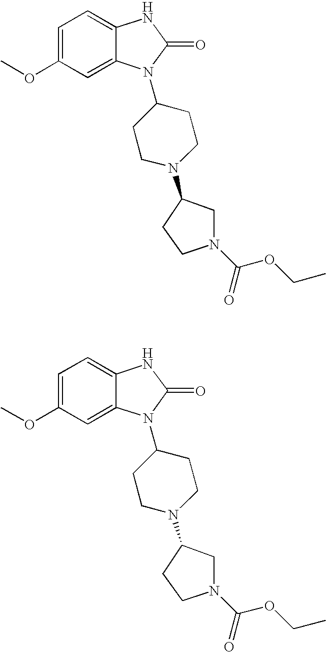

Following the procedure described in Example 30 and 35, the title compounds were prepared from tert-butyl 4-aminopiperidine-1-carboxylate, 4-methoxy-2-fluoronitrobenzene and ethyl 3-oxopyrrolidine-1-carboxylate.

Isomer 1 (Example 39). 1H NMR (400 MHz, METHANOL-D4): δ ppm 1.23 (t, J=7.03 Hz, 3H) 1.65-1.87 (m, 3H) 2.09-2.30 (m, 3H) 2.38-2.59 (m, 2H) 2.79-3.04 (m, 2H) 3.13 (t, J=9.77 Hz, 2H) 3.24-3.36 (m, 1H) 3.56 (t, J=9.57 Hz, 1H) 3.65-3.73 (m, 1H) 3.77 (s, 3H) 4.09 (q, J=7.03 Hz, 2H) 4.20-4.35 (m, 1H) 6.61 (dd, J=8.59, 1.95 Hz, 1H) 6.92 (d, J=8.59 Hz, 1H) 6.99 (d, J=1.95 Hz, 1H). MS (M+1): 389.2

Isomer 2 (Example 40). ¹H NMR (400 MHz, METHANOL-D4) δ ppm 1.23 (t, J=7.03 Hz, 3H) 1.65-1.87 (m, 3H) 2.09-2.30 (m, 3H) 2.38-2.59 (m, 2H) 2.79-3.04 (m, 2H) 3.13 (t, J=9.77 Hz, 2H) 3.24-3.36 (m, 1H) 3.56 (t, J=9.57 Hz, 1H) 3.65-3.73 (m, 1H) 3.77 (s, 3H) 4.09 (q, J=7.03 Hz, 2H) 4.20-4.35 (m, 1H) 6.61 (dd, J=8.59, 1.95 Hz, 1H) 6.92 (d, J=8.59 Hz, 1H) 6.99 (d, J=1.95 Hz, 1H). MS (M+1): 389.2

### Example 41

### Ethyl (3S)-3-[4-(6-cyano-2-oxo-2,3-dihydro-1H-benzimidazol-1-yl)piperidin-1-yl]pyrrolidine-1-carboxylate

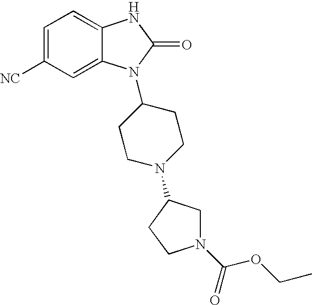

### Step A. The preparation of tert-butyl (3S)-3-(4-oxopiperidin-1-yl)pyrrolidine-1-carboxylate

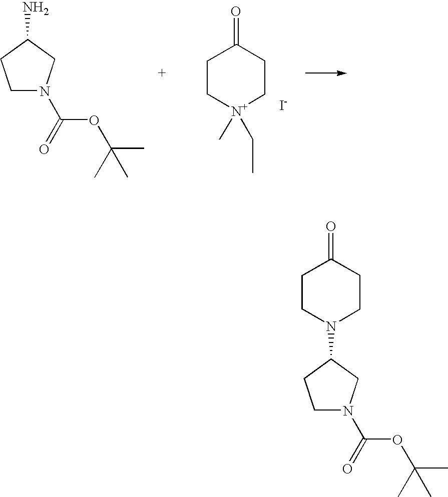

To a stirred and boiling mixture of (S)-1-boc-3-aminopyrrolidine (1 g, 5.37 mmol), potassium carbonate (742 mg, 5.37 mmol) and ethanol 920 mL) was added drop wise over a period of 15 min a solution of N-ethyl-N-methyl-4-oxo-piperidinium (2 g, 7.39 mmol) in water (10 mL). The reaction mixture was refluxed for 20 min, subsequently poured into water (50 mL) and 3 N NaOH solution (50 mL) was added. The mixture was partitioned between EtOAc/H2O. The aqueous was further extracted with EtOAc (×2). The combined extracts were dried with Na2SO4, filtered and evaporated. The residue was purified by high pH HPLC to afford the title compound. tert-Butyl (3S)-3-(4-oxopiperidin-1-yl)pyrrolidine-1-carboxylate was obtained as white solid (1.40 g, 98% yield). ¹H NMR (400 MHz, CHLOROFORM-D): δ ppm 1.49 (S, 9H), 1.69 (m, 1H), 1.75-1.92 (m, 1H), 2.06-2.16 (m, 1H), 2.36-2.53 (m, 3H), 2.55-2.88 (m, 4H), 2.88-3.02 (m, 1H), 3.15 (q, J=10.03 Hz, 1H), 3.23-3.39 (m, 1H), 3.45-3.82 (m, 2H).

### Step B. The preparation of tert-butyl (3S)-3-(4-aminopiperidin-1-yl)pyrrolidine-1-carboxylate

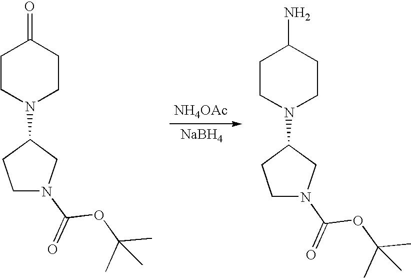

To a mixture of tert-Butyl (3S)-3-(4-oxopiperidin-1-yl)pyrrolidine-1-carboxylate (200 mg, 0.75 mmol), ammonium acetate (575 mg, 7.5 mmol) and sodium borohydride (56.7 mg, 1.5 mmol) was added methanol (10 mL) at RT. The reaction mixture was stirred at RT for 12 hrs and concentrated in vacuo and the residue was diluted with brine. The aqueous phase was extracted with CH₂Cl₂ (3×). The combined organic phases was dried over MgSO₄, filtered, and concentrated in vacuo. The residue was purified by high pH HPLC to afford the title compound. tert-Butyl (3S)-3-(4-aminopiperidin-1-yl)pyrrolidine-1-carboxylate was obtained as colorless oil (51% yield). ¹H NMR (400 MHz, CHLOROFORM-D): δ ppm 1.20-1.50 (m, 10H), 1.52-1.95 (m, 4H), 1.97-2.32 (m, 3H), 2.59-2.98 (m, 3H), 3.02-3.15 (m, 1H), 3.18-3.33 (m, 1H), 3.41-3.84 (m, 3H).

### Step C. The preparation of Ethyl (3S)-3-[4-(6-cyano-2-oxo-2,3-dihydro-1H-benzimidazol-1-yl)piperidin-1-yl]pyrrolidine-1-carboxylate

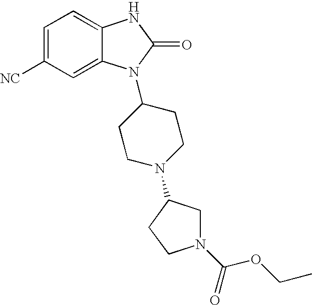

Following the procedure described in Example 30 Step A & B and Example 15, the title compound was prepared from tert-Butyl (3S)-3-(4-aminopiperidin-1-yl)pyrrolidine-1-carboxylate, 4-cyano-2-fluoronitrobenzene and ethyl chloroformate (44% yield). ¹H NMR (400 MHz, METHANOL-D4): δ ppm 1.24 (t, J=7.23 Hz, 3H), 1.68-1.93 (m, 3H), 2.12-2.38 (m, 3H), 2.41-2.59 (m, 2H), 2.87-3.09 (m, 2H), 3.11-3.25 (m, 2H), 3.26-3.42 (m, 1H), 3.53-3.63 (m, 1H), 3.66-3.78 (m, 1H), 4.10 (q, J=7.29 Hz, 2H), 4.25-4.42 (m, 1H), 7.15 (d, J=8.20 Hz, 1H), 7.34-7.47 (m, 1H), 7.80 (s, 1H). MS (M+1): 384.2

### Example 42

### Ethyl (3S)-3-[4-(6-chloro-2-oxo-2,3-dihydro-1H-benzimidazol-1-yl)piperidin-1-yl]pyrrolidine-1-carboxylate

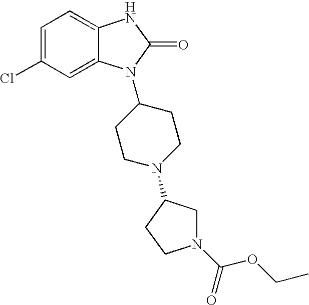

Following the procedure described in Example 41, the title compound was prepared from tert-Butyl (3S)-3-(4-aminopiperidin-1-yl)pyrrolidine-1-carboxylate, 4-chloro-2-fluoronitrobenzene and ethyl chloroformate (16% yield). ¹H NMR (400 MHz, METHANOL-D4): δ ppm 1.17 (t, J=7.23 Hz, 3H), 1.61-1.86 (m, 3H), 2.08-2.30 (m, 3H), 2.33-2.51 (m, 2H), 2.82-3.32 (m, 5H), 3.44-3.59 (m, 1H), 3.60-3.73 (m, 1H), 3.98-4.08 (m, 2H), 4.15-4.31 (m, 1H), 6.89-6.93 (m, 1H), 6.94-6.98 (m, 1H), 7.40 (d, J=1.95 Hz, 1H). MS (M+1): 393.2

### Example 43

### Ethyl (3S)-3-[4-(6-trifluoromethyl-2-oxo-2,3-dihydro-1H-benzimidazol-1-yl)piperidin-1-yl]pyrrolidine-1-carboxylate

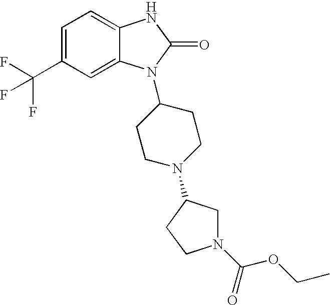

### Step A. The preparation of tert-butyl (3S)-3-{[(benzyloxy)carbonyl]amino}pyrrolidine-1-carboxylate

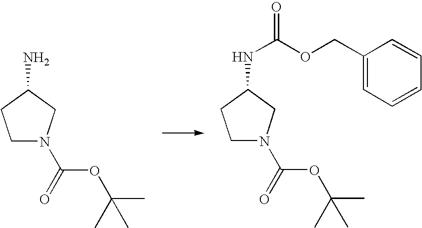

To (S)-tert-butyl 3-aminopyrrolidine-1-carboxylate (4.99 g, 26.8 mmol) and trimethylamine (5.6 mL, 40.2 mmol) in DCM (20 mL) was added benzyl carbonochloridate compound (7.28 g, 26.80 mmol) in DCM (10 mL) slowly at 0° C. After 3 hrs reaction, H₂O was added to the mixture. The aq layer was back extracted with dichloromethane (10 mL) (3×). Combined the organic layers were washed with brine, organic layer dried over MgSO4, filtered and concentrated. The residue was purified by high pH HPLC to afford the title compound as colorless oil (3.40 g, 40% yield).

### Step B. The preparation of ethyl (3S)-3-aminopyrrolidine-1-carboxylate

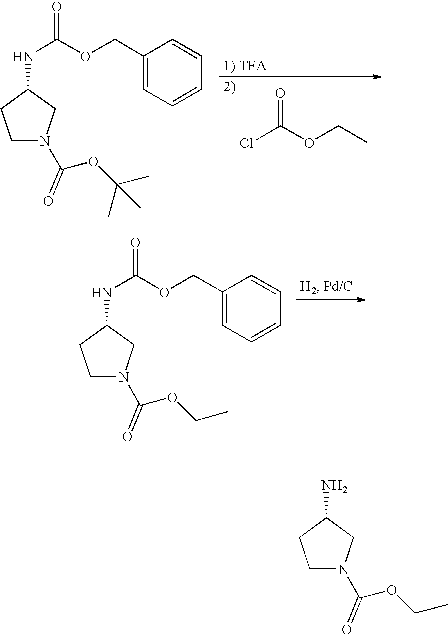

Following the procedure described in Example 15, the intermediate ethyl (3S)-3-{[(benzyloxy)carbonyl]amino}pyrrolidine-1-carboxylate was prepared from (3S)-3-{[(benzyloxy)carbonyl]amino}pyrrolidine-1-carboxylate and ethylchloroformate. A solution of ethyl (3S)-3-{[(benzyloxy)carbonyl]amino}pyrrolidine-1-carboxylate (2.23 g, 7.63 mmol) prepared above in methanol (20 mL) was stirred in the presence of Palladium on Charcoal (30 mg) under a hydrogen atmosphere for 12 hrs. The reaction mixture was filtered over Celite and concentrated to give title compound (1.19 g, 98% yield).

### Step C. The preparation of ethyl (3S)-3-(4-oxopiperidin-1-yl)pyrrolidine-1-carboxylate

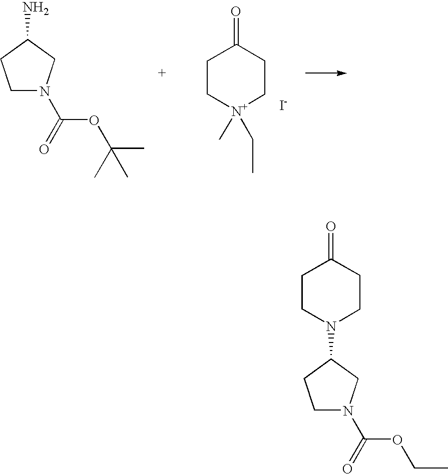

Following the procedure described in Example 41 Step A, the intermediate ethyl (3S)-3-(4-oxopiperidin-1-yl)pyrrolidine-1-carboxylate was prepared from ethyl (3S)-3-aminopyrrolidine-1-carboxylate and N-ethyl-N-methyl-4-oxo-piperidinium iodide (1.66 g, 98% yield). ¹H NMR (400 MHz, CHLOROFORM-D) δ ppm 1.05 (t, J=7.23 Hz, 1H), 1.27 (t, J=7.03 Hz, 3H), 1.76-1.95 (m, 1H), 2.06-2.20 (m, 1H), 2.34-2.52 (m, 3H), 2.57-2.88 (m, 4H), 2.90-3.07 (m, 1H), 3.14-3.43 (m, 2H), 3.52-3.85 (m, 2H), 4.14 (q, J=7.03 Hz, 2H).

### Step D. The preparation of ethyl (3S)-3-(4-{[2-amino-5-(trifluoromethyl)phenyl]amino}piperidin-1-yl)pyrrolidine-1-carboxylate

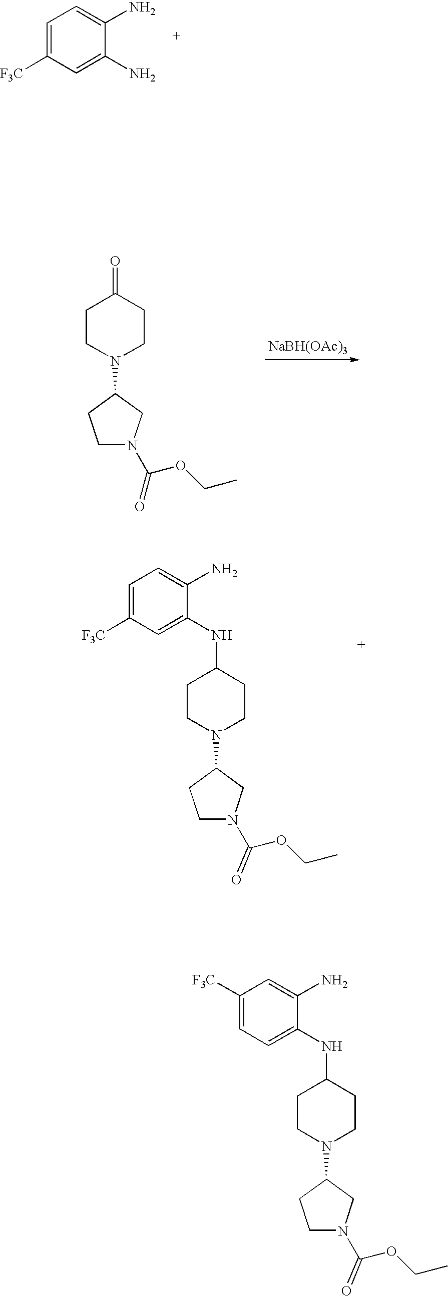

To 4-(trifluoromethyl)benzene-1,2-diamine (220 mg, 1.25 mmol) and (S)-ethyl 3-(4-oxopiperidin-1-yl)pyrrolidine-1-carboxylate (300 mg, 1.25 mmol) in CH₂Cl₂ (10 mL) was added sodium triacetoxyborohydride (794 mg, 3.75 mmol) followed by acetic acid (0.357 mL, 6.24 mmol) at 25° C. After 3 hrs reaction, water was added to the mixture. The aqueous layer was back extracted with DCM (10 mL) (3×). Combined the organic layers were washed with brine, the organic layer was dried over MgSO4, filtered and concentrated. The crude material was used for next reaction without purification.

### Step E. The preparation of ethyl (3S)-3-[4-(6-trifluoromethyl-2-oxo-2,3-dihydro-1H-benzimidazol-1-yl)piperidin-1-yl]pyrrolidine-1-carboxylate

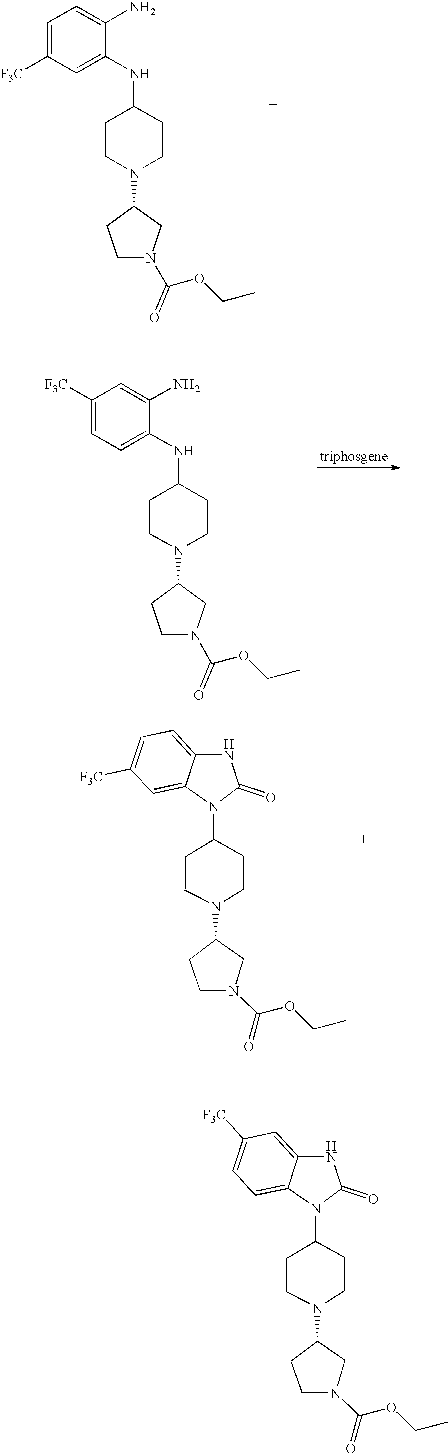

To (S)-ethyl 3-(4-(2-amino-5-(trifluoromethyl)phenylamino)piperidin-1-yl)pyrrolidine-1-carboxylate, (S)-ethyl 3-(4-(2-amino-4-(trifluoromethyl)phenylamino)piperidin-1-yl)pyrrolidine-1-carboxylate prepared in step D and triethylamine (0.261 mL, 1.875 mmol) in DCM (5 mL) was added triphosgene (0.136 g, 0.458 mmol) in DCM (1 mL) slowly at 0° C. After 0.5 hr reaction, water was added to the mixture. The aqueous layer was back extracted with dichloromethane (10 mL) (3×) and combined organic layers were washed with brine. The organic layer was dried over MgSO4, filtered and concentrated. The residue was purified on a high pH HPLC to provide a mixture of two products in a ratio of 3:1 (49.7 mg). This solid was purified again by chiral HPLC (Chiral AD column, 10% isopropanol in hexane). The first fraction was obtained as the title compound (19.7 mg. Ethyl (3S)-3-[4-(6-trifluoromethyl-2-oxo-2,3-dihydro-1H-benzimidazol-1-yl)piperidin-1-yl]pyrrolidine-1-carboxylate was obtained as white solid (21.3 mg, 7.36% yield). ¹H NMR (400 MHz, METHANOL-D4): δ ppm 1.20-1.27 (m, 3H), 1.70-1.89 (m, 3H), 2.08-2.37 (m, 3H), 2.43-2.61 (m, 2H), 2.82-3.11 (m, 2H), 3.11-3.24 (m, 2H), 3.25-3.42 (m, 1H), 3.51-3.64 (m, 1H), 3.67-3.81 (m, 1H), 4.10 (q, J=7.03 Hz, 2H), 4.29-4.44 (m, 1H), 7.16 (d, J=8.20 Hz, 1H), 7.35 (d, J=8.20 Hz, 1H), 7.71 (s, 1H). MS (M+1): 427.2

### Example 44

### Ethyl (3S)-3-[4-(5-trifluoromethyl-2-oxo-2,3-dihydro-1H-benzimidazol-1-yl)piperidin-1-yl]pyrrolidine-1-carboxylate

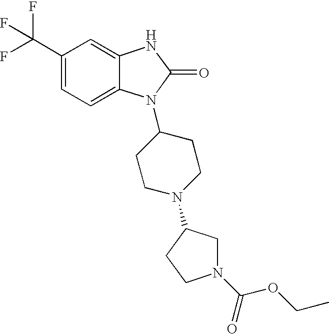

The second fraction from Example 43 was obtained as the title compound (12.7 mg, 4.8% yield). ¹H NMR (400 MHz, METHANOL-D4): δ ppm 1.21-1.28 (m, 3H), 1.74-1.91 (m, 3H), 2.14-2.38 (m, 3H), 2.43-2.59 (m, 2H), 2.88-3.10 (m, 2H), 3.12-3.25 (m, 2H), 3.26-3.41 (m, 1H), 3.52-3.63 (m, 1H), 3.66-3.77 (m, 1H), 4.10 (q, J=7.03 Hz, 2H), 4.26-4.39 (m, 1H), 7.29 (s, 1H), 7.35 (d, J=8.20 Hz, 1H), 7.49 (d, J=8.20 Hz, 1H). MS (M+1): 427.2

### Example 45

### Ethyl (3S)-3-[4-(6-tert-butyl-2-oxo-2,3-dihydro-1H-benzimidazol-1-yl)piperidin-1-yl]pyrrolidine-1-carboxylate

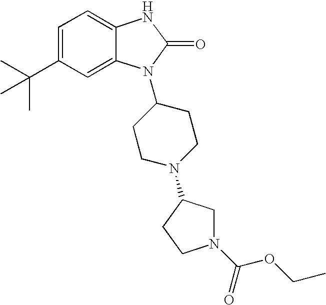

Following the procedure described in Example 43, the title compound and its regio isomer were prepared from 4-(tert-butyl)benzene-1,2-diamine and (S)-ethyl 3-(4-oxopiperidin-1-yl)pyrrolidine-1-carboxylate in a ratio of 1:3. This solid was purified by chiral AD HPLC (10% isopropanol in hexane), and the first fraction (minor fraction) was obtained as the title compound. ¹H NMR (400 MHz, METHANOL-D4): δ ppm 1.24 (t, J=7.23 Hz, 3H), 1.31-1.37 (s, 9H), 1.71-1.93 (m, 3H), 2.16-2.38 (m, 3H), 2.43-2.63 (m, 2H), 2.88-3.11 (m, 2H), 3.14-3.25 (m, 2H), 3.26-3.43 (m, 1H), 3.54-3.65 (m, 1H), 3.68-3.82 (m, 1H), 4.10 (q, J=7.16 Hz, 2H), 4.25-4.43 (m, 1H), 6.95 (d, J=8.20 Hz, 1H), 7.09 (dd, J=8.40, 1.76 Hz, 1H), 7.41 (s, 1H). MS (M+1): 415.3

### Example 46

### Ethyl (3S)-3-[4-(5-tert-butyl-2-oxo-2,3-dihydro-1H-benzimidazol-1-yl)piperidin-1-yl]pyrrolidine-1-carboxylate

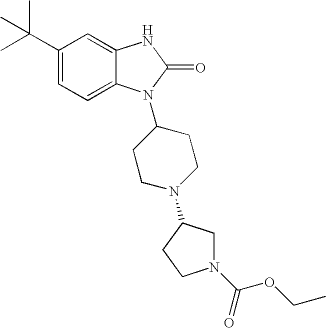

The second fraction (major fraction) from Example 45 was obtained as the title compound. ¹H NMR (400 MHz, METHANOL-D4): δ ppm 1.24 (t, J=7.23 Hz, 3H), 1.30 (s, 9H), 1.67-1.92 (m, 3H), 2.12-2.35 (m, 3H), 2.40-2.59 (m, 2H), 2.85-3.08 (m, 2H), 3.11-3.24 (m, 2H), 3.24-3.41 (m, 1H), 3.52-3.63 (m, 1H), 3.65-3.80 (m, 1H), 4.10 (q, J=7.03 Hz, 2H), 4.19-4.36 (m, 1H), 7.07-7.13 (m, 2H), 7.23-7.29 (m, 1H). MS (M+1): 415.3

### Example 47

### Ethyl (3S)-3-[4-(6-trifluoromethoxy-2-oxo-2,3-dihydro-1H-benzimidazol-1-yl)piperidin-1-yl]pyrrolidine-1-carboxylate

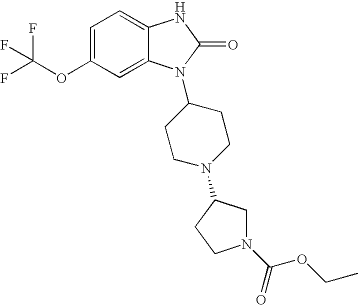

Following the procedure described in Example 43, the title compound and its regio isomer were prepared from 4-(trifluoromethoxy)benzene-1,2-diamine and (S)-ethyl 3-(4-oxopiperidin-1-yl)pyrrolidine-1-carboxylate in a ratio of 1:6. This regioisomeric mixture was purified on chiral AD HPLC (10% isopropanol in hexane), and the first fraction (minor fraction) was obtained as the title compound. ¹H NMR (400 MHz, METHANOL-D4): δ ppm 1.24 (t, J=7.23 Hz, 3H), 1.70-1.93 (m, 3H), 2.13-2.36 (m, 3H), 2.39-2.60 (m, 2H), 2.86-3.09 (m, 2H), 3.12-3.25 (m, 2H), 3.26-3.41 (m, 1H), 3.52-3.63 (m, 1H), 3.67-3.78 (m, 1H), 4.10 (q, J=7.29 Hz, 2H), 4.24-4.40 (m, 1H), 6.90-6.99 (m, 1H), 7.02-7.12 (m, 1H), 7.39 (s, 1H). MS (M+1): 443.2.

### Example 48

### Ethyl (3S)-3-[4-(5-trifluoromethoxy-2-oxo-2,3-dihydro-1H-benzimidazol-1-yl)piperidin-1-yl]pyrrolidine-1-carboxylate

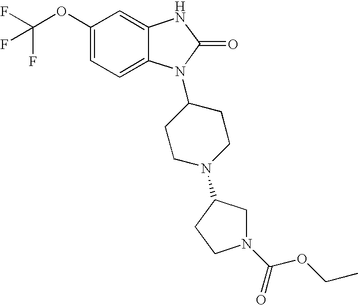

The second fraction (major fraction) from Example 47 was obtained as the title compound. ¹H NMR (400 MHz, METHANOL-D4): δ ppm 1.23 (t, J=7.03 Hz, 3H), 1.71-1.92 (m, 3H), 2.10-2.34 (m, 3H), 2.40-2.57 (m, 2H), 2.84-3.08 (m, 2H), 3.09-3.22 (m, 2H), 3.25-3.38 (m, 1H), 3.51-3.62 (m, 1H), 3.65-3.77 (m, 1H), 4.09 (q, J=7.03 Hz, 2H), 4.20-4.36 (m, 1H), 6.90-7.00 (m, 2H), 7.37 (d, J=8.59 Hz, 1H). MS (M+1): 443.2

### Example 49

### Ethyl (3S)-3-[4-(5-fluoro-2-oxo-2,3-dihydro-1H-benzimidazol-1-yl)piperidin-1-yl]pyrrolidine-1-carboxylate

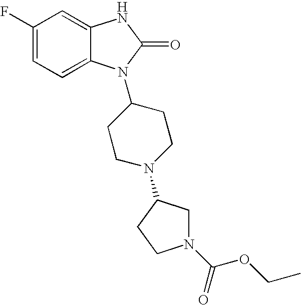

Following the procedure described in Example 43, the title compound was prepared from 4-fluorobenzene-1,2-diamine and (S)-ethyl 3-(4-oxopiperidin-1-yl)pyrrolidine-1-carboxylate to give a mixture of regio isomers in a ratio of 3:2. The regioisomeric mixture was purified by chiral AD HPLC (10% isopropanol in hexane containing 0.1% diethylamine), and the second fraction was obtained as the title compound. ¹H NMR (400 MHz, METHANOL-D4): δ ppm 1.25 (t, J=7.13 Hz, 3H), 2.03-2.30 (m, 3H), 2.40-2.56 (m, 1H), 2.70-2.90 (m, 2H), 3.18-3.35 (m, 2H), 3.38-3.60 (m, 2H), 3.62-3.82 (m, 3H), 3.87-4.03 (m, 2H), 4.13 (q, J=7.10 Hz, 2H), 4.42-4.62 (m, 1H), 6.77-6.88 (m, 2H), 7.19 (dd, J=8.69, 4.20 Hz, 1H). MS (M+1): 377.2

1. A compound of formula IA, a pharmaceutically acceptable salt thereof, diastereomer, enantiomer, or mixture thereof: 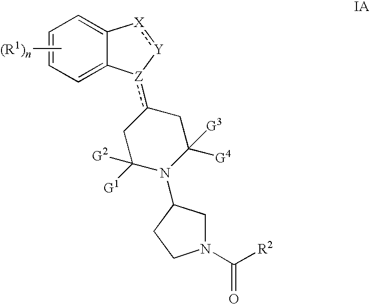
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl;
n is 1, 2, 3 or 4;
each R is independently hydrogen, C₁₋₆alkyl, C₂₋₆alkenyl or halogenated C₁₋₆alkyl; and
X, Y and Z are independently selected from C(═O), NH, N—CH₃, N, C, CH₂, and CH, wherein at least one of X, Y and Z is selected from NH, N—CH₃ and N; wherein at most one of X, Y and Z is C(═O); and wherein Z is not C(═O).

2. A compound as claimed in claim 1, wherein
R² is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

3. A compound as claimed in claim 1, wherein
R² is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

4. A compound as claimed in claim 1, wherein
R¹ is selected from hydrogen, halogen, methyl, ethyl, —CN, —C(═O)—NH₂, —CO₂CH₃, —CO₂H, hydroxyl, methoxy, ethoxy, isopropoxy, trifluoromethyl, FCH₂—, F₂CH—, CHF₂O—, and CF₃O—.

5. A compound as claimed in claim 1, wherein
Z is selected from N, C and CH.

6. A compound as claimed in claim 1, wherein Y is selected from N and C(═O).

7. A compound as claimed in claim 1, wherein X is selected from CH₂, NH and N—CH₃.

8. A compound as claimed in claim 1, wherein G¹, G², G³ and G⁴ are independently selected from —H and methyl.

9. A compound as claimed in claim 1, wherein G¹, G², G³ and G⁴ are —H.

10. A compound as claimed in claim 1, wherein G² and G³ are linked together to form an ethylene, and G¹ and G⁴ are independently selected from —H and methyl.

11. A compound of formula IIA, a pharmaceutically acceptable salt thereof, diastereomer, enantiomer, or mixture thereof: 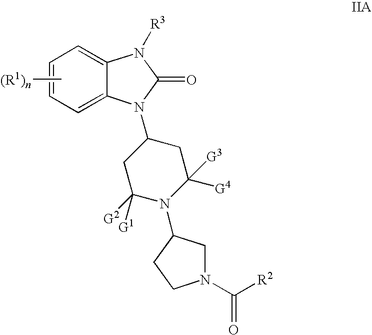
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
R³ is H or C₁₋₄ alkyl;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

12. A compound as claimed in claim 11, wherein R¹ of formula IIA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, F₂CH—, CF₃O—, and CHF₂O—.

13. A compound as claimed in claim 11, wherein R¹ of formula IIA is selected from hydrogen halogen, —CN, methoxy and C₁₋₃alkyl.

14. A compound as claimed in claim 11, wherein R² of formula IIA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

15. A compound as claimed in claim 11, wherein R² of formula IIA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

16. A compound of formula IIIA, a pharmaceutically acceptable salt thereof, diastereomer, enantiomer, or mixture thereof: 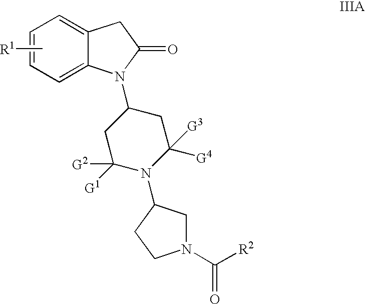
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

17. A compound as claimed in claim 16, wherein R¹ of formula IIIA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, F₂CH—, CF₃O—, and CHF₂O—.

18. A compound as claimed in claim 16, wherein R¹ of formula IIIA is selected from hydrogen, halogen, —CN, methoxy and C₁₋₃alkyl.

19. A compound as claimed in claim 16, wherein R² of formula IIIA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

20. A compound as claimed in claim 16, wherein R² of formula IIIA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

21. A compound of formula IVA, a pharmaceutically acceptable salt thereof, diastereomer, enantiomer, or mixture thereof: 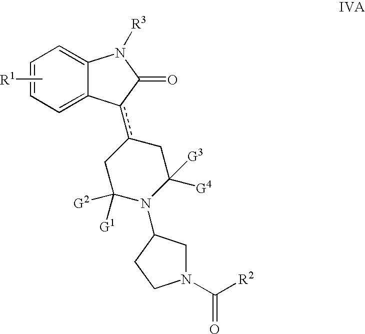
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
R³ is H or C₁₋₄ alkyl;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

22. A compound as claimed in claim 21, wherein R¹ of formula IVA is independently selected from hydrogen, halogen, C₁₋₃alkyl, —CN, —C(═O)—OH, —C(═O)—NH₂, hydroxy, methoxy, ethoxy, isopropoxy, trifluoromethyl, FCH₂—, F₂CH—, CF₃O—, and CHF₂O—.

23. A compound as claimed in claim 21, wherein R¹ of formula IVA is selected from hydrogen halogen, —CN, methoxy and C₁₋₃alkyl.

24. A compound as claimed in claim 21, wherein R² of formula IVA is selected from hydrogen, C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy, wherein said C₁₋₆alkyl, C₂₋₆alkenyl, C₁₋₆alkoxy, C₁₋₆alkylamino, di-C₁₋₆alkylamino, C₂₋₉heteroaryl, C₃₋₆heterocycloalkyl-C₁₋₃alkyl and benzyloxy are optionally substituted by one or more groups selected from amino, halogen, hydroxy, C₁₋₆alkoxy and —CN.

25. A compound as claimed in claim 21, wherein R² of formula IVA is selected from hydrogen, C₁₋₄alkyl, C₁₋₄alkoxy, C₃₋₆heterocycloalkyl-C₁₋₃alkyl, C₃₋₆cycloalkyl, C₃₋₆heterocycloalkyl, C₁₋₄alkylamino, di-C₁₋₄alkylamino, C₄₋₆heteroaryl and benzyloxy.

26. A compound selected from
Ethyl 3-[4-(2-oxo-2,3-dihydro-1H-indol-1-yl)piperidin-1-yl]pyrrolidine-1-carboxylate;
Ethyl 3-[4-(2-oxo-2,3-dihydro-1H-benzimidazol-1-yl)piperidin-1-yl]pyrrolidine-1-carboxylate;
Ethyl 3-[4-(5-chloro-2-oxo-2,3-dihydro-1H-benzimidazol-1-yl)piperidin-1-yl]pyrrolidine-1-carboxylate;
Benzyl 3-[4-(2-oxo-2,3-dihydro-1H-benzimidazol-1-yl)piperidin-1-yl]pyrrolidine-1-carboxylate;
t-Butyl 3-[4-(2-oxo-2,3-dihydro-1H-benzimidazol-1-yl)piperidin-1-yl]pyrrolidine-1-carboxylate;
Isopropyl 3-[4-(2-oxo-2,3-dihydro-1H-benzimidazol-1-yl)piperidin-1-yl]pyrrolidine-1-carboxylate;
1-[1-(1-butyrylpyrrolidin-3-yl)piperidin-4-yl]-1,3-dihydro-2H-benzimidazol-2-one;
N,N-dimethyl-3-[4-(2-oxo-2,3-dihydro-1H-benzimidazol-1-yl)piperidin-1-yl]pyrrolidine-1-carboxamide;
1-{1-[1-(3-methylbutanoyl)pyrrolidin-3-yl]piperidin-4-yl}-1,3-dihydro-2H-benzimidazol-2-one;
Ethyl 3-[4-(3-methyl-2-oxo-2,3-dihydro-1H-benzimidazol-1-yl)piperidin-1-yl]pyrrolidine-1-carboxylate;
Ethyl 3-[4-(1H-1,2,3-benzotriazol-1-yl)piperidin-1-yl]pyrrolidine-1-carboxylate;
Ethyl 3-[4-(2-oxo-1,2-dihydro-3H-indol-3-ylidene)piperidin-1-yl]pyrrolidine-1-carboxylate;
Ethyl 3-[4-(2-oxo-2,3-dihydro-1H-indol-3-yl)piperidin-1-yl]pyrrolidine-1-carboxylate;
tert-Butyl (3S)-3-[4-(2-oxo-2,3-dihydro-1H-benzimidazol-1-yl)piperidin-1-yl]pyrrolidine-1-carboxylate;
Ethyl (3S)-3-[4-(2-oxo-2,3-dihydro-1H-benzimidazol-1-yl)piperidin-1-yl]pyrrolidine-1-carboxylate;
Ethyl (3R)-3-[4-(2-oxo-2,3-dihydro-1H-benzimidazol-1-yl)piperidin-1-yl]pyrrolidine-1-carboxylate;
Methyl (3S)-3-[4-(2-oxo-2,3-dihydro-1H-benzimidazol-1-yl)piperidin-1-yl]pyrrolidine-1-carboxylate;
iso-Propyl (3S)-3-[4-(2-oxo-2,3-dihydro-1H-benzimidazol-1-yl)piperidin-1-yl]pyrrolidine-1-carboxylate;
1-{1-{(3S)-1-(cyclopentylcarbonyl)pyrrolidin-3-yl}piperidin-4-yl}-1,3-dihydro-2H-benzimidazol-2-one;
1-(1-{(3S)-1-[(2S)-tetrahydrofuran-2-ylcarbonyl]pyrrolidin-3-yl}piperidin-4-yl)-1,3-dihydro-2H-benzimidazol-2-one;
1-(1-{(3S)-1-[(1-methyl-1H-pyrrol-2-yl)carbonyl]pyrrolidin-3-yl}piperidin-4-yl)-1,3-dihydro-2H-benzimidazol-2-one;
1-(1-{(3S)-1-[4-(2-oxopyrrolidin-1-yl)butanoyl]pyrrolidin-3-yl}piperidin-4-yl)-1,3-dihydro-2H-benzimidazol-2-one;
1-(1-{(3S)-1-[3-(2-oxopyrrolidin-1-yl)propanoyl]pyrrolidin-3-yl}piperidin-4-yl)-1,3-dihydro-2H-benzimidazol-2-one;
1-methyl-3-(1-{(3S)-1-[3-(2-oxopyrrolidin-1-yl)propanoyl]pyrrolidin-3-yl}piperidin-4-yl)-1,3-dihydro-2H-benzimidazol-2-one;
(3S)—N-ethyl-3-[4-(2-oxo-2,3-dihydro-1H-benzimidazol-1-yl)piperidin-1-yl]pyrrolidine-1-carboxamide;
Ethyl (3S)-3-[4-(2-oxo-2,3-dihydro-1H-indol-1-yl)piperidin-1-yl]pyrrolidine-1-carboxylate;
Ethyl (3R)-3-[4-(2-oxo-2,3-dihydro-1H-indol-1-yl)piperidin-1-yl]pyrrolidine-1-carboxylate;
Methyl (3S)-3-[4-(2-oxo-2,3-dihydro-1H-indol-1-yl)piperidin-1-yl]pyrrolidine-1-carboxylate;
1-(1-{(3S)-1-[3-(2-oxopyrrolidin-1-yl)propanoyl]pyrrolidin-3-yl}piperidin-4-yl)-1,3-dihydro-2H-indol-2-one;
Ethyl 3-[3-(2-oxo-2,3-dihydro-1H-benzimidazol-1-yl)-8-azabicyclo[3.2.1]oct-8-yl]pyrrolidine-1-carboxylate;
Ethyl 3-[4-(7-fluoro-2-oxo-2,3-dihydro-1H-benzimidazol-1-yl)piperidin-1-yl]pyrrolidine-1-carboxylate;
Ethyl 3-[4-(5-fluoro-2-oxo-2,3-dihydro-1H-benzimidazol-1-yl)piperidin-1-yl]pyrrolidine-1-carboxylate;
Ethyl 3-[4-(4-fluoro-2-oxo-2,3-dihydro-1H-benzimidazol-1-yl)piperidin-1-yl]pyrrolidine-1-carboxylate;
Ethyl 3-[4-(6-fluoro-2-oxo-2,3-dihydro-1H-benzimidazol-1-yl)piperidin-1-yl]pyrrolidine-1-carboxylate;
(3S) Ethyl 3-[4-(6-fluoro-2-oxo-2,3-dihydro-1H-benzimidazol-1-yl)piperidin-1-yl]pyrrolidine-1-carboxylate;
(3R) Ethyl 3-[4-(6-fluoro-2-oxo-2,3-dihydro-1H-benzimidazol-1-yl)piperidin-1-yl]pyrrolidine-1-carboxylate;
(3S) Ethyl 3-[4-(6-methyl-2-oxo-2,3-dihydro-1H-benzimidazol-1-yl)piperidin-1-yl]pyrrolidine-1-carboxylate;
(3R) ethyl 3-[4-(6-methyl-2-oxo-2,3-dihydro-1H-benzimidazol-1-yl)piperidin-1-yl]pyrrolidine-1-carboxylate;
(3S) Ethyl 3-[4-(6-methoxy-2-oxo-2,3-dihydro-1H-benzimidazol-1-yl)piperidin-1-yl]pyrrolidine-1-carboxylate;
(3R) ethyl 3-[4-(6-methoxy-2-oxo-2,3-dihydro-1H-benzimidazol-1-yl)piperidin-1-yl]pyrrolidine-1-carboxylate;
Ethyl (3S)-3-[4-(6-cyano-2-oxo-2,3-dihydro-1H-benzimidazol-1-yl)piperidin-1-yl]pyrrolidine-1-carboxylate;
Ethyl (3S)-3-[4-(6-chloro-2-oxo-2,3-dihydro-1H-benzimidazol-1-yl)piperidin-1-yl]pyrrolidine-1-carboxylate;
Ethyl (3S)-3-[4-(6-trifluoromethyl-2-oxo-2,3-dihydro-1H-benzimidazol-1-yl)piperidin-1-yl]pyrrolidine-1-carboxylate;
Ethyl (3S)-3-[4-(5-trifluoromethyl-2-oxo-2,3-dihydro-1H-benzimidazol-1-yl)piperidin-1-yl]pyrrolidine-1-carboxylate;
Ethyl (3S)-3-[4-(6-tert-butyl-2-oxo-2,3-dihydro-1H-benzimidazol-1-yl)piperidin-1-yl]pyrrolidine-1-carboxylate;
Ethyl (3S)-3-[4-(5-tert-butyl-2-oxo-2,3-dihydro-1H-benzimidazol-1-yl)piperidin-1-yl]pyrrolidine-1-carboxylate;
Ethyl (3S)-3-[4-(6-trifluoromethoxy-2-oxo-2,3-dihydro-1H-benzimidazol-1-yl)piperidin-1-yl]pyrrolidine-1-carboxylate;
Ethyl (3S)-3-[4-(5-trifluoromethoxy-2-oxo-2,3-dihydro-1H-benzimidazol-1-yl)piperidin-1-yl]pyrrolidine-1-carboxylate;
Ethyl (3S)-3-[4-(5-fluoro-2-oxo-2,3-dihydro-1H-benzimidazol-1-yl)piperidin-1-yl]pyrrolidine-1-carboxylate;
and pharmaceutically acceptable salts thereof.

27-30. (canceled)

31. A pharmaceutical composition comprising a compound according to claim 1 and a pharmaceutically acceptable carrier.

32. A method for the therapy of pain in a warm-blooded animal, comprising the step of administering to said animal in need of such therapy a therapeutically effective amount of a compound according claim 1.

33. A method for the therapy of Alzheimer's disease in a warm-blooded animal, comprising the step of administering to said animal in need of such therapy a therapeutically effective amount of a compound according to claim 1.

34. A method for the therapy of schizophrenia in a warm-blooded animal, comprising the step of administering to said animal in need of such therapy a therapeutically effective amount of a compound according to claim 1.

35. A process for preparing a compound of Formula IA, comprising: 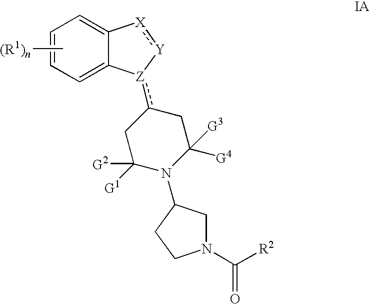
reacting a compound of Formula VA with a compound of formula VI, 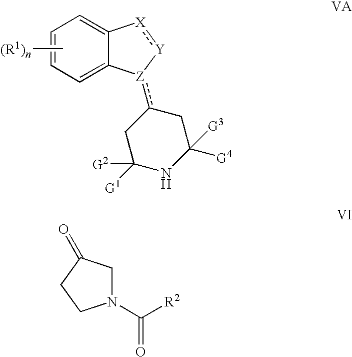
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

36. A compound of formula VIIA, 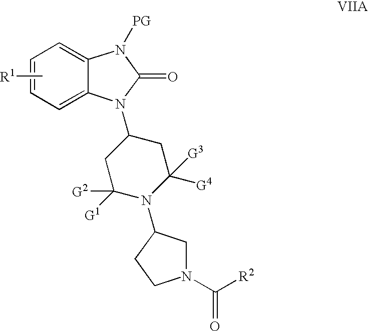
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl;
each R is independently hydrogen, C₁₋₆alkyl, C₂₋₆alkenyl or halogenated C₁₋₆alkyl; and
PG is selected from —C(═O)═O-t-Bu and —C(═O)—OBn.

37. A method for the therapy of anxiety in a warm-blooded animal, comprising the step of administering to said animal in need of such therapy a therapeutically effective amount of a compound according to claim 1.

38. A method for the therapy of depression in a warm-blooded animal, comprising the step of administering to said animal in need of such therapy a therapeutically effective amount of a compound according to claim 1.

39. A compound of formula VIII, 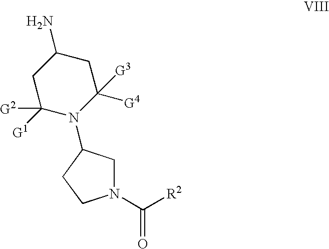
wherein
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

40. A compound of formula IX 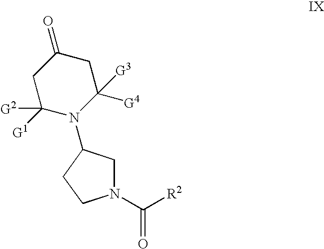
wherein
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

41. A process for preparing a compound of Formula VIII, comprising: 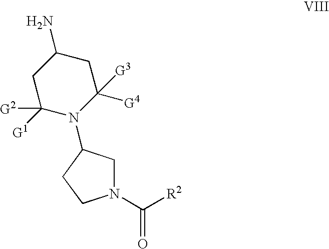
reductive amination of a compound of Formula IX 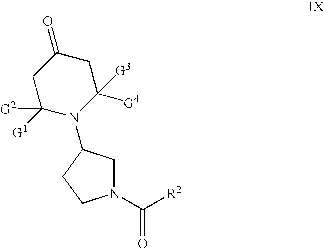
wherein
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

42. A method of preparing a compound of formula IIA comprising 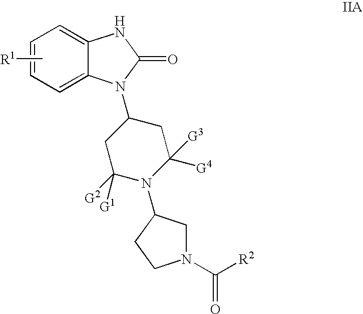
a first of step reacting a compound of formula IX 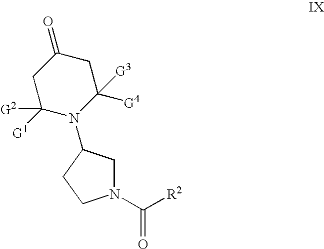
with a compound of formula X in the presence of a reducing agent to form a first product; and 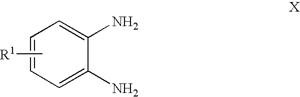
reacting said first product with a phosgene type reagent to form the compound of formula IIA wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl; and
each R is independently hydrogen, C₁₋₆alkyl, C₂₋₆alkenyl or halogenated C₁₋₆alkyl.

43. A method of preparing a compound of formula IIA comprising 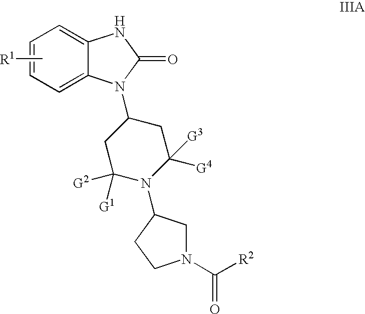
a first step of reacting a compound of formula VIII 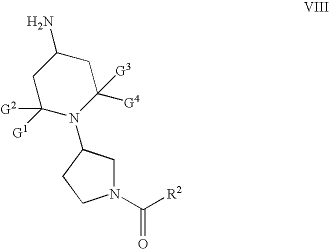
with a compound of formula XI in the presence of a reducing agent to form a first product containing a nitro group; and 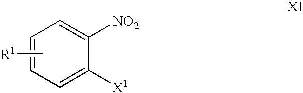
reducing the nitro group of said first product into an amino group to form a second product;
reacting said second product with a phosgene type reagent to form the compound of formula IIA
wherein
R¹ is independently selected from hydrogen, halogen, C₁₋₆alkyl, C₂₋₆alkenyl, —CN, —C(═O)—OR, —C(═O)—NR₂, hydroxy, C₁₋₆alkoxy, trifluoromethyl, FCH₂—, F₂CH—, CHF₂O—, CF₃O—, C₆₋₁₀aryl, and C₂₋₉heteroaryl;
R² is selected from hydrogen, C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy, wherein said C₁₋₆alkyl, C₂₋₆alkenyl, C₁₋₆alkoxy, C₁₋₆alkylamino, di-C₁₋₆alkylamino, C₆₋₁₀aryl, C₆₋₁₀aryloxy, C₂₋₉heteroaryl, C₂₋₉heteroaryloxy, C₃₋₅heterocycloalkyloxy, C₃₋₉heterocycloalkyl, C₆₋₁₀aryl-C₁₋₃alkoxy, C₆₋₁₀aryl-C₁₋₃alkyl, C₂₋₉heteroaryl-C₁₋₃alkoxy, C₂₋₉heteroaryl-C₁₋₃alkyl, C₃₋₆heterocycloalkyl-C₁₋₃alkoxy, C₃₋₆heterocycloalkyl-C₁₋₃alkyl, C₃₋₉cycloalkyl, C₃₋₆cycloalkyloxy, and C₃₋₆cycloalkyl-C₁₋₃alkyl, C₃₋₆cycloalkyl-C₁₋₃alkoxy are optionally substituted with one or more group selected from —CN, —SR, —OR, —O(CH₂)ₚ—OR, R, —C(═O)—R, —CO₂R, —SO₂R, —SO₂NR₂, halogen, —NO₂, —NR₂, —(CH₂)ₚNR₂, and —C(═O)—NR₂;
G¹, G², G³ and G⁴ are independently selected from H and methyl; or two of G¹, G², G³ and G⁴ are linked together to form a C₁₋₄alkylene, and the other two are independently selected from H and methyl;
X¹ is a halogen; and
each R is independently hydrogen, C₁₋₆alkyl, C₂₋₆alkenyl or halogenated C₁₋₆alkyl.
